(19) United States
(12) Patent Application Publication
(10) Pub. No.: US 2010/0292306 A1
(43) Pub. Date: Nov. 18, 2010

(54) Compositions And Methods For The Treatment Of Muscular Dystrophy

(76) Inventors: C. George Carlson, Kirksville, MO (US); Rajvir Singh, New Haven, CT (US)

Correspondence Address: LATHROP & GAGE LLP, 4845 PEARL EAST CIRCLE, SUITE 201, BOULDER, CO, 80301, US

(21) Appl. No.: 12/845,558

(22) Filed: Jul. 28, 2010

Related U.S. Application Data
(63) Continuation-in-part of application No. 11/439,714, filed on May 24, 2006.
(60) Provisional application No. 60/684,504, filed on May 24, 2005.
(60) Provisional application No. 60/762,394, filed on Jan. 26, 2006.
(60) Provisional application No. 61/229,197, filed on Jul. 28, 2009.

(51) Int. Cl. A61K 31/713; A61K 31/5375; A61K 31/575; A61K 31/40; A61K 31/655; A61K 31/343; A61P 21/00

(52) U.S. Cl. 514/44 A; 514/231.2; 514/182; 514/423; 514/156; 514/468

(57) ABSTRACT

Compositions and methods for treatment of individuals diagnosed with a dystrophin deficiency are disclosed. In particular, inhibitors of NFκB transactivation and/or inhibitors that suppress p65 expression are used to prevent and/or reverse muscle damage in animals or humans lacking dystrophin. Such compositions and methods are useful in the treatment of individuals with muscular dystrophy.

### RELATED APPLICATIONS

This application claims priority to U.S. Provisional Application 61/229,197 filed Jul. 28, 2009, the contents of which are hereby incorporated into this application by reference. This application is also a continuation-in-part of U.S. patent application Ser. No. 11/439,714 filed May 24, 2006, which claims priority to U.S. Provisional Application No. 60/684,504 filed May 24, 2005, and U.S. Provisional Application No. 60/762,394 filed Jan. 26, 2006, the contents of which are hereby incorporated into this application by reference.

### BACKGROUND

1. Field of the Invention

The present invention relates to pharmaceutical compositions and methods for the treatment of muscular dystrophies.

2. Description of the Related Art

Muscular dystrophies (MD) are a group of genetic diseases that afflict more than 50,000 Americans. The diseases are characterized by progressive weakness and degeneration of the skeletal muscle fibers that control movement. Both voluntary and involuntary muscles, such as heart and respiratory muscles, are replaced by fat and connective tissue in the late stages of the disease. Muscular dystrophies are heterogeneous disorders.

Muscular dystrophies are heterogeneous in that the causes of the disorders are diverse. One of the most common forms of muscular dystrophy is Duchenne muscular dystrophy (DMD), which afflicts about 1 out of every 3500 males. DMD is characterized by a near complete lack of dystrophin production, which is typically caused by mutations in the gene coding for the dystrophin protein. While some females may carry the mutations without showing symptoms of the disease, DMD usually progresses rapidly in males. Patients with severe DMD may lose the ability to walk by age 12, and their respiratory system may stop functioning by approximately age 20 which usually results in death. In a less debilitating form of DMD, also known as Becker MD, dystrophin production is not shut down completely, but is reduced. For most DMD, the age of onset and rate of progression depends on how much dystrophin is produced and how well it functions in the cells.

There is currently no cure for muscular dystrophies, but medications and therapy may slow the progress of the disease. Respiratory therapy, physical therapy to prevent painful muscle contractures, orthopedic appliances used for support, and corrective orthopedic surgery may be needed to improve a patient's quality of life. Other treatments may include cardiac pacemakers and pharmaceuticals aimed at treating individual symptoms, for example corticosteroids can slow the rate of muscle deterioration, mild anesthetics can reduce pain, and antiepileptics can prevent seizures. Many of these treatments are ineffective and have severe side effects. There is therefore a need for a therapy that can prevent or slow the progress of muscular dystrophy with no or relatively milder side effects.

### SUMMARY OF THE INVENTION

The instrumentalities disclosed herein advance the art by providing a method for administering a pharmaceutical composition comprising an inhibitor of the nuclear factor kappa B (NFkappaB or NFκB) pathway in an amount that can inhibit or reduce the activation of NFκB in a subject diagnosed with muscular dystrophy. The present compositions and methods may be used to treat, prevent or reverse muscle damage or wasting caused by muscular dystrophy. More particularly, the disclosed compositions and methods are suitable for treating the form of muscular dystrophy caused by dystrophin deficiency.

In one aspect, this disclosure pertains to a method of administering a pharmaceutical composition. The methods may include diagnosing a subject that is in need of treatment for muscular dystrophy, administering to the subject an inhibitor of NFκB activation in an amount effective to inhibit nuclear activation of NFκB in said subject, and permitting the inhibitor to achieve therapeutic benefit for muscular dystrophy in the subject. By way of example, the NFκB inhibitor may include pyrrolidine dithiocarbamate, curcumin (diferuloylmethane), or their combinations.

NFκB plays an important role in the transcription activation of a large number of genes. For instance, many cytokines genes are activated by NFκB. It is shown here that the levels of some cytokines, such as IL-1β, IL-6 and TNFα, are elevated in the muscle of the mdx mouse model of muscular dystrophy. In another aspect of the present invention, chemicals or biological agents may be used to inhibit or reduce the production or secretion of these cytokines, and thus prevent or slow muscle degeneration in MD patients.

In one aspect, an improved method is provided for treating muscular dystrophy in a subject by administering to the subject an agent in an amount that is effective in decreasing the level or the activity of the p65 subunit of NFκB in the muscular tissues of the subject. The agents may include but are not limited to NF-κB transactivation inhibitors, p65 expression inhibitors, IκB stabilizing agent, Ikk inhibitors or combination thereof. More preferably, the agent is an inhibitor of p65 expression. In one embodiment, the agent decreases the translation from p65 mRNA into p65 protein. In another aspect, the agent may be a small interfering RNA (siRNA) that complements the nucleic acid coding sequence of the p65 subunit of NFκB. In another aspect, the agent may decrease the transcription of the p65 gene into p65 mRNA. In yet another aspect, the agent may decrease the transactivation activity of the p65 subunit of NFκB in the muscular tissues of the subject. Examples of such an agent may include but are not limited to ursodeoxycholic acid (UDCA) or gossypol. UDCA is preferred.

In one aspect, the agent of the present disclosure may be administered as a pharmaceutical composition containing the agent as well as at least one pharmaceutically inactive ingredient. In another aspect, the agent may be administered to the subject at an amount that is sufficient to decrease the level or the activity of the p65 subunit of NFκB by at least 30%, or more preferably, by at least 50% in the muscular tissues of the subject.

In an embodiment of the present disclosure a subject diagnosed with Duchenne mucular dystrophy may be treated with an agent that is a specific translation blocking vivo-morpholino to decrease the level or the activity of the p65 subunit of NFκB in the muscular tissues of the subject.

In another embodiment of the present disclosure a subject diagnosed with Duchenne mucular dystrophy may be treated with an agent that reduces cellular p65 levels by de-stabilizing cellular p65 in dystrophic tissues thereby promoting its degradation and/or cytosolic localization. The agent may include but is not limited to an inhibitor of the peptidyl prolyl isomerase (PIN-1; NCBI Accession CAG28582, AAH02899) that stabilizes the expression of p65 in dystrophic muscle fibers.

In another embodiment of the present disclosure, the method of treatment may be enhanced by monitoring the effects of treatment, and adjusting treatment by increasing, reducing, or temporarily stopping treatment based on the result of monitoring. For instance, NFκB levels in the subject may be monitored to ascertain the status and effect of treatment. The total number of muscle fibers in skeletal muscles in the subject that are subjected to passive stretch during normal use may be monitored in order to ascertain the effect of treatment. In addition, the whole body strength of the subject may be measured during the course of the treatment.

Other parameters that may be monitored include the total tension generated by isolated muscles in the limbs of dystrophic subjects, the percentage of total cellular NFκB that is localized to the nuclear compartment of isolated dystrophic skeletal muscle, electrical properties and resting membrane potential of isolated dystrophic skeletal muscle fibers, the number of surviving striated muscle fibers in isolated skeletal muscles that are subjected to passive stretch during normal use, the total number of muscle fibers in skeletal muscles that are subjected to passive stretch during normal use, the number of skeletal muscle nuclei per muscle fiber in skeletal muscles that are subjected to passive stretch during normal use, the cross-sectional area of individual dystrophic muscle fibers in certain regions of skeletal muscle fibers that are subjected to passive stretch during normal use, the percentage of centrally located nuclei in muscle fibers that are subjected to passive stretch during normal use, and the total tension generated by isolated muscles in the limbs of dystrophic subjects.

In other aspects, the NFκB pathway is well documented in the art, and various inhibitors are available to regulate this pathway at one or more loci of pathway events. For example, an inhibitor may work by stabilizing the IκB protein and thereby preventing the NFκB from translocating into the nucleus. Another inhibitor may regulate the protein level of NFκB itself, yet other inhibitors may regulate the NFκB pathway by modulating the activity of nuclear NFκB.

As an alternative treatment method, a composition for use in the treatment of muscular dystrophy may contain a first inhibitor of NFκB activation in an amount that is effective to inhibit NFκB activation in the muscle cells of a subject, where the inhibitor of NFκB activation is effective to down-regulate the NFκB pathway at a predetermined first level. A second inhibitor of NFκB activation may then be used in an amount that is effective to inhibit NFκB activation in the muscle cells of a subject. The second inhibitor of NFκB activation is effective to down-regulate the NFκB pathway at a predetermined second level. Such predetermined second level is preferably different from the predetermined first level.

The two inhibitors may act on the same or different proteins in the NFκB pathway. In this manner, possible chronic side-effect of long term treatment may be mitigated by adjusting the ratio of the first and second inhibitors at intervals during a course of treatment. Adjustment may be on a regular periodic basis as specific cellular pathways regulating gene activation are modulated by the treatment and the particular drug combination becomes less efficacious, or as needed by assessment according to the aforementioned monitoring program.

In yet another embodiment, a subject may be treated with an inhibitor of NFκB activation in a first amount that is effective in bringing down the level of NFκB activation to a first level. After a period of treatment, a different amount of the same NFκB inhibitor is administered such that the level of NFκB activation is changed to a second level that is different from the first level achieved during the previous treatment period. In this manner, possible chronic side-effect of long term treatment may be mitigated by adjusting the level of NFκB inhibition. Adjustment may be on a regular periodic basis as specific cellular pathways regulating gene activation are modulated by the treatment and the particular drug combination becomes less efficacious, or as needed by assessment according to the aforementioned monitoring program.

### BRIEF DESCRIPTION OF THE DRAWINGS

FIG. 1 including component parts a, b, c, and d thereof shows that acute in vivo PDTC administration increases cytosolic IκB-α levels in the mdx diaphragm (Western blot using anti-polyclonal IkB-α, #sc-371 antibody; Santa Cruz Biotechnology, Santa Cruz Calif.).

FIG. 2 including component parts A, B, C, and D thereof compares the morphology of freshly isolated and fixed triangularis sterni (TS) muscles from age-matched control mdx (A, B) and PDTC-treated mdx mice (C, D).

FIG. 3 demonstrates that the methods used to assess the percent fibers and percent striated fibers in different regions of the mdx TS muscle provide an excellent determination of the loss of muscle fibers and the loss of striated muscle fibers in the dystrophic TS muscle.

FIG. 4 including component parts A, B and C thereof shows that daily treatment with PDTC (50-75 mg/kg ip; 27-30 days) increases the density of striated fibers in the TS of mdx mice aged 8.5-9 months at sacrifice (Series 1 experiments).

FIG. 5 including component parts A1, A2, A3, A4, B1, B2, B3 and B4 thereof shows that daily treatment with PDTC (50 mg/kg ip; 48-76 days) increases the density of fibers in the TS of mature mdx mice aged 11.5-18.5 months (A, Series 2) and 21.5-22 months (B, Series 3) at sacrifice.

FIG. 6 including component parts A1, A2, A3, A4, B1, B2, B3 and B4 thereof demonstrates that daily PDTC treatment increases the density of striated TS fibers in mdx mice aged 11.5-18.5 months (A, Series 2) and 21.5-22 months (B, Series 3) at sacrifice.

FIG. 7 including component parts A, b and C thereof presents representative cross-sections (20 μm calibration) obtained from nondystrophic TS muscles (A) and from TS muscles from adult mdx mice treated chronically with vehicle (B) or PDTC (C).

FIG. 8 including component parts A1, A2, A3, B1, B2, B3, C1, C2, and C3 thereof shows average histograms of fiber diameter for non-dystrophic TS muscles (A1-A3), TS muscles from mdx mice treated chronically with vehicle (B1-B3) and TS muscles from mdx mice treated chronically with PDTC (C1-C3).

FIG. 9 shows the average fiber diameters obtained for each of the 3 TS regions in nondystrophic, vehicle-injected mdx, and PDTC-injected mdx mice.

FIG. 10 shows the average fiber density for the 3 different regions of mature mdx TS muscles treated chronically either with vehicle or PDTC.

FIG. 11 shows that chronic treatment of an mdx mouse with PDTC for a period of 8.5 months decreases the loss of muscle fibers observed in the mdx TS muscle between 5 and 13.5 months.

FIG. 12 shows the average numbers of myonuclei per fiber cross section obtained from mature nondystrophic (ND) mice, mature mdx mice treated chronically with vehicle (mdx-veh) and mature mdx mice treated chronically with PDTC (mdx-PDTC).

FIG. 13 shows that percent centronucleation is enhanced in mature mdx TS muscle fibers and significantly decreased by chronic treatment with PDTC.

FIG. 14 shows that a 30 day treatment with 50 mg/kg PDTC beginning at 30 days of age significantly reduces the percent centronucleation observed in the mdx TS muscle at 60 days of age.

FIG. 15 including component parts A and B thereof shows that Gd³⁺-sensitive resting Ca²⁺ currents are not responsible for a significant depression in resting potential observed in adult mdx TS muscle fibers.

FIG. 16 shows that daily administration of PDTC restores the resting potential to non-dystrophic levels in mice aged 8.5-9 months at sacrifice (Series 1 experiments).

FIG. 17 including component parts A1, A2, B1, and B2 thereof shows that daily treatment with PDTC (50 mg/kg ip; 48-76 days) significantly increases the resting potential of fibers in the TS of mature mdx mice at 12.9 months (A, Series 2) and at 20 months (B, Series 3). N=number of impaled fibers, number of TS muscles. Shown are means and SE.

FIG. 18 presents measurements of forward pulling tensions (FPTs) produced by an mdx mouse using the noninvasive “whole body tension” measurement (Carlson and Makiejus, 1990).

FIG. 19 including component parts A and B thereof shows that the decline in forward pulling tension represented by the top 10 pulling attempts provides a measure of weakness in the mdx mouse that can be assessed noninvasively before and after a chronic period of drug administration.

FIG. 20 including component parts A and B thereof demonstrates that chronic PDTC treatment reduces the fatigue index (FI) in the mdx mouse (Series 2 experiments).

FIG. 21 shows that PDTC treatment produces functional improvement by significantly increasing whole body strength in mature mdx mice.

FIG. 22 shows that daily treatment with PDTC prevents a decline in functional reserve (FR) normally seen in developing, young adult mdx mice.

FIG. 23 shows the Gastrocnemius Twitch amplitudes at Iₒ in nondystrophic (A) and mdx mice that were administered daily injections of saline (B) or PDTC (C).

FIG. 24 shows that daily treatment with PDTC improves twitch tension development in mature mdx mice (average age 15 months at sacrifice).

FIG. 25 shows that daily treatment with PDTC improves twitch tension development in young adult mdx mice.

FIG. 26 including component parts A, B and C thereof uses trans AM assays of NFκB to show that chronic treatment with PDTC reduces total cellular NFκB (A) and increases the proportion of total cellular NFκB in the cytosolic fraction (B) in gastrocnemius muscle preparations. (C) shows the corresponding proportions in the nuclear fractions.

FIG. 27 including component parts A1, A2, B1, B2, and C1 thereof shows that the cytosolic extracts of dystrophic (mdx) diaphragm exhibit elevated levels of NFκB dependent cytokines.

FIG. 28 including comparative Gels 1 and 2 thereof shows that a single in vivo treatment with sulfasalazine (“SS”, Sigma Number S-0883; 100 mg/kg dissolved in HEPES Ringer solution and administered by intraperitoneal injection) may reduce nuclear NF-kappaB activation in the costal diaphragm of the mdx mouse.

FIG. 29 including comparative Gels 1 and 2 thereof shows that a single in vivo treatment with parthenolide (PTN, Sigma Number P-0667; 5 mg/kg dissolved in HEPES Ringer solution containing 0.1% dimethylsulfoxide (DMSO) and administered by intraperitoneal injection) may reduce nuclear NFκB activation in the costal diaphragm of the mdx mouse.

FIG. 30 shows that TNFα expression in costal diaphragm is reduced following a single injection of sulfasalazine (SS; 100 mg/kg, ip) administered 3 hours prior to euthanasia.

FIG. 31 shows that the expression of IL1-1β in cytosolic extracts of mdx muscle depends upon the muscle origin and is not influenced by a single injection of sulfasalazine(SS; 100 mg/kg, ip) administered 3 hours prior to euthanasia.

FIG. 32 shows that the expression of IL6 in cytosolic extracts of mdx muscle depends upon the muscle origin and is reduced by a single injection of sulfasalazine (SS; 100 mg/kg, ip) administered 3 hours prior to euthanasia.

FIG. 33 shows that daily treatment with sulfasalazine (SS; 100 mg/kg; intraperitoneal, 68 days) significantly improves the resting membrane potential in the TS muscle.

FIG. 34 including component parts A, B and C thereof shows portions of individual records of forward pulling tensions (FPTs) which illustrate the top 10 FPTs exerted during typical WBT recording sessions.

FIG. 35 including component parts A, B and C thereof shows that Mdx mice exhibit significant deficits in FPT that begin at 30 days of age and continue throughout the mdx lifespan.

FIG. 36 including component parts A and B thereof shows that examination of the decline in FPT over the top 10 FPTs indicates that mdx mice exhibit a greater proportional decline in FPT per maximal forward pulling maneuver than nondystrophic mice.

FIG. 37 shows that Mdx mice exhibit significantly larger PDP values than age-matched nondystrophic mice.

FIG. 38 including component parts A, B and C thereof shows that long term treatment of young adult mdx mice with UDCA increases WBT5 (A), WBT10 (B), and reduces nuclear p65 activation (C) in the mdx costal diaphragm.

FIG. 39 including component parts A and B thereof shows that nuclear extracts from limb and respiratory musculature exhibit significant increases in nuclear p65 activation.

FIG. 40 including component parts A and B thereof shows Increases in whole cell expression of IκB-α in mdx costal diaphragm.

FIG. 41 including component parts A, B and C thereof shows increases in the whole cell expression of p65 in mdx costal diaphragm.

FIG. 42 including component parts A, B and C thereof shows increases in the cytosolic and nuclear expression of p65 in mdx costal diaphragm.

FIG. 43 including component parts A and B thereof shows increases in the whole cell expression of p50 in mdx costal diaphragm.

FIG. 44 including component parts A, B1, B2, C1 and C2 thereof shows alterations in the whole cell expression of IKKα and IKKβ in mdx costal diaphragm.

FIG. 45 including component parts A1, A2, B, and C thereof shows the expression of phosphorylated IKKα (ser 176), total IKKα, and IKKγ in mdx costal diaphragm.

FIG. 46 including component parts A1, A2, B, C, D and E thereof shows increases in the whole cell expression of alternative NF-κB pathway signaling components in the mdx costal diaphragm.

FIG. 47 including component parts A, B and C thereof shows alterations in the expression of NIK, phospho-NIK, and TRAF-3 in mdx costal diaphragms.

FIG. 48 including component parts A1, A2, B1, B2, C1 and C2 thereof shows the influence of proteasomal inhibition on the cytosolic expression of IκB-α and phospho-IκB-α in nondystrophic and mdx costal diaphragm.

FIG. 49 including component parts A1, A2 and B thereof shows SUMO expression and sumoylation of IκB-α in nondystrophic and mdx costal diaphragm.

FIG. 50 including component parts A and B thereof shows increases in whole cell Akt phosphorylation in mdx costal diaphragm.

### DETAILED DESCRIPTION

The design of a more effective treatment for Duchenne and related dystrophinopathies depends upon an improved understanding of the pathogenesis of these disorders. Since the initial discovery of dystrophin, considerable attention has been placed on understanding those mechanisms by which the absence of this cytoskeletal protein in Duchenne muscular dystrophy and in the dystrophic (mdx) mouse (Bulfield et al., 1984, Hoffman et al., 1987 and Koenig and Kunkel, 1990) ultimately leads to muscle necrosis. A primary hypothesis based upon the membrane localization of dystrophin (Arahata et al., 1988) and the subsequent characterization of the transmembrane dystrophin-glycoprotein complex (Ervasti and Camp<ell, 1991) suggests that dystrophin forms a structural bridge that supports the plasmalemma by physically interacting with extracellular components of the basal lamina (Matsumura and Camp<ell, 1994). An alternative hypothesis suggests that alterations in ion channel function and associated local increases in Ca²⁺ influx may occur when ion channels aggregate in association with a dystrophic cytoskeleton (Carlson, 1998). Each of these hypotheses generally involve secondary increases in Ca²⁺ influx that occur as a result of the structural breakdown of the plasma membrane or by the formation of abnormal ion channel—cytoskeletal interactions at specific ion channel aggregates (Carlson, 1998).

Muscular Dystrophy (MD) is neuro-muscular disease with a diverse range of manifestation and pathogenesis. The diagnosis of MD may thus utilize a wide range of clinical tools. Although behavioral diagnosis may be the primary tool to spot the disease, neurological, histological, biochemical, or genetic testings may be used to more definitively diagnose the disease (See generally, El-Bohy and Wong, 2005). Physical methods such as microwave or NMR imaging may also aid a clinician in the diagnosis of MD. The compositions and methods disclosed herein may be used to treat or to slow the progress of a patient who has been diagnosed with Muscular Dystrophy. These compositions may also be useful for a subject who may not have the typical symptoms of MD but who is otherwise in need of such a therapy. By way of example, the compositions and methods may prove useful for a subject who may be genetically predisposed to MD based on family history but who may not have yet developed any symptoms of MD. Such person may be treated in a prophylactic way to prevent or delay the onset of symptomatic disease.

Although fluorometric and electrophysiological investigations provided evidence for enhanced resting Ca²⁺ influx in cultured dystrophic (mdx) myotubes (Carlson et al., 2001 and Tutdibi et al., 1999), Mn²⁺ quench determinations did not indicate differences in resting Ca²⁺ influx between nondystrophic and mdx dissociated adult flexor digitorum brevis muscle fibers (DeBacker et al., 2002). Results obtained in this laboratory also indicated that extrajunctional resting Ca²⁺ influx was not elevated in severely dystrophic and intact undissociated adult mdx skeletal muscle fibers (Carlson et al., 2003), and demonstrated that extrajunctional increases in resting Ca²⁺ influx are therefore not pathogenic in muscular dystrophy. These investigations also showed that the mdx triangularis sterni (TS) muscle exhibits severe dystrophic alterations with fibrosis, fat infiltration, hypercontraction, dissolution of myofibrillar material, cytoplasmic rarefaction with delta lesions, and substantial decreases in the number of striated muscle fibers and in the total number of muscle fibers. This loss of muscle fibers continues until the mdx TS becomes a thin layer of connective tissue with only a few remaining muscle fibers by about 1.5-2 years of age (Carlson et al., 2003).

Functional studies indicate that the TS is an expiratory muscle that is chronically passively stretched to about 107% of its resting length and concentrically activated at a rate of approximately 250 times per minute (DeTroyer and Ninane, 1986, Hwang et al., 1989, Gosselin et al., 2003 and Ninane et al., 1989). The severe dystrophy seen in this mdx muscle therefore strongly suggests that physical factors or signaling pathways activated by passive stretch play central roles in the pathogenesis of dystrophic muscle (Carlson et al., 2003). Such factors and/or pathways would presumably also be involved in the susceptibility of dystrophic fibers to the damaging effects of eccentric muscle contractions (Petrof et al., 1993 and Weller et al., 1990).

A potential pathway that may be involved in stretch-dependent dystrophic pathogenesis (Carlson et al., 2003) is suggested from results described by Kumar and Boriek (2003), who showed that a single 15-min period of passive stretch increased the nuclear activation of the transcription factor NFκB by two times in isolated nondystrophic muscle fibers, and that resting mdx muscle fibers from 15-day-old mice exhibited nuclear NFκB levels that were approximately two times those seen in age-matched nondystrophic fibers. The stretch-dependent increase in nuclear activation of NFκB in nondystrophic muscle did not require Ca²⁺ influx and was associated with a reduction in cytosolic levels of IκB-α secondary to the activation of IκB kinase (IKK; Kumar and Boriek, 2003). These results are consistent with stretch-dependent activation of the classical NFκB pathway in which IKK phosphorylates IκB-α, thus tagging it for future ubiquitination and proteasomal degradation (Karin et al., 2004). This process disassociates IκB-α from the dimeric p50/p65 NFκB, thus allowing the dimer to enter the nucleus and activate an array of NFkappaB-dependent genes. These genes include several proinflammatory cytokines (e.g., IL-1B, Il-2, IL-6, IL-8, TNF-α), chemokines (IL-8, RANTES), inducible enzymes (iNOS, cyclooxygenase), and adhesion molecules (ICAM, VCAM; Barnes, 1997, Li et al., 2002 and Siebenlist et al., 1994). Because some of the genes activated by NFκB encode proteins that are beneficial to the muscles and promote cell division and cell survival (e.g.cyclin D1, bcl-2, bclₓₗ, cellular inhibitor of apoptosis 1 (cIAP1), cIAP2, xIAP), while other genes encode proteins that are pro-inflammatory, the elevated NFκB activity may be either compensatory or detrimental to the structure and function of dystrophic skeletal muscle. The effects of NFκB activation in dystrophic muscle may be determined by chronically inhibiting the NFκB pathway and determining the effects of this inhibition on the structure and function of dystrophic skeletal muscle.

NFκB is a transcription factor that plays an important role in many cellular processes. In its inactive state, NFκB resides in the cytoplasm and is bound to another protein called IκB. Upon cell activation, IκB may be modified and targeted for degradation. The freed NFκB may then translocate into the nucleus, and along with other transcription factors, activate transcription of target genes. (See Karin et al., 2004, for a general discussion of the NFκB pathway).

NFκB activation refers to a state of the NFκB molecule that is capable of participating in transcription activation. Inhibitors of NFκB activation generally refer to an agent that either partially or completely blocks NFκB participation in the activation of many its target genes. A large number of NFκB target genes have been reported in the literature. The mRNAs of these target genes are normally present at low levels and their levels increase dramatically when NFκB and other transcription factors bind to regulatory elements of these genes and activate their transcription.

To evaluate the effect of various chemical agents on animal models of MD, experimental male and female mdx mice may be injected intraperitoneally (ip) with various chemicals at different dosage. In the case of pyrrolidine dithiocarbamate (PDTC) (50-75 mg/kg of PDTC (obtained from Sigma) may be dissolved in a saline solution (HEPES-Ringer; in mM: 147.5 NaCl, 5 KCl, 2 CaCl₂, 11 glucose, 5 HEPES, pH 7.35) for injection. More particularly, mdx mice aged 5-22 months at the beginning of the treatment may receive daily injections of PDTC at doses between 50 and 75 mg/kg for a period of 27-30 consecutive days. The mice may be sacrificed after 1-24 months from the beginning of treatment to determine the effect of the treatment with the chemicals.

In one embodiment, the treatment effect may be evaluated by comparing the morphological results between 3 regions of the triangularis sterni (TS); the caudal third of the muscle extending toward the xiphoid process, the middle third, and the cephalad third of the muscle. This procedure is adopted because there are obvious differences between these regions in the extent of pathology and the muscle thickness in the mdx TS (not clearly seen in the nondystrophic TS) even when examining the muscle at low power (e.g., 4×). Results from animals that die during the experimental time frame (both saline injected and PDTC treated) are typically excluded from the study. Although the current sample of PDTC-treated mice is not sufficient to determine any toxic effects of the drug, no obvious effect of PDTC treatment on viability is observed in the overall sample of mice used in these studies.

Conventional intracellular recording techniques may be used to determine resting membrane potential in individual TS muscle fibers from nondystrophic (C57BI 10SnJ) and mdx (C57BI10-mdx) mice (For detailed description, see Carlson and Roshek, 2001). After removing the TS muscle preparation (See Carlson et al., 2003), the entire TS muscle may be placed in a specialized recording chamber and stretched across a thin glass coverslip using specialized dissecting hooks (manufactured locally) that are attached to the sternum and to small (1-2 mm) cut sections of the ribs. The preparation may be placed in the chamber with the external surface of the TS facing upwards and is minimally stretched to its approximate length in situ (about 95-105% of resting length).

The chamber is typically filled with a small volume (2 ml) of normal HEPES Ringer solution at room temperature and fiber-filled glass micropipettes (3 M KCl; R=20-70 MΩ) may be used to impale individual fibers at an angle of 90° to the principal fiber axis. Signals may be amplified with a Warner Instruments Model IE 201 electrometer and displayed on an oscilloscope. Individual fibers may be viewed using an Olympus IMT2F microscope equipped with long working distance (20×, 40×) objectives. Impalements may be obtained after first electrically balancing the recording system (0 mV output relative to ground) and viewing the electrode tip over a muscle fiber. The electrode may be slowly advanced and inserted into the muscle fiber by gently tapping the manipulator or temporarily unbalancing the negative capacitance of the recording circuit. The voltage deflection associated with membrane insertion is noted, and each recording may be maintained for a few minutes before the electrode is rapidly withdrawn from the fiber. The voltage deflection associated with withdrawal from the cell is also noted and the larger of the two deflections (i.e., insertion or withdrawal) is usually taken as the fiber resting potential. Differences between the insertion and withdrawal voltage deflections are generally 0-4 mV.

Resting potentials from approximately 20 fibers may be obtained from each isolated TS muscle over a total recording period of about 1.5 h. The presence or absence of miniature endplate potentials is also noted to identify endplate from nonendplate regions. When no attempt is made to identify endplates, approximately 97% of the recordings may be from nonendplate regions.

Morphological assessment of total fiber density and density of striated fibers may be conducted as described in the following text. Immediately after completing the resting potential measurements, the minimally stretched TS muscle preparations attached to the dissecting hooks may be fixed overnight in 2% glutaraldehyde (0.1 M cacodylate buffer) and subsequently washed several times in 0.1 M cacodylate. Before removing the preparation from the recording chamber, microphotographs of approximately 24 randomly sampled areas may be obtained at 200-300× magnification in caudal, middle, and cephalad regions of the TS. In the initial studies, a smaller number of photographs may be obtained over the middle portion of the TS muscle.

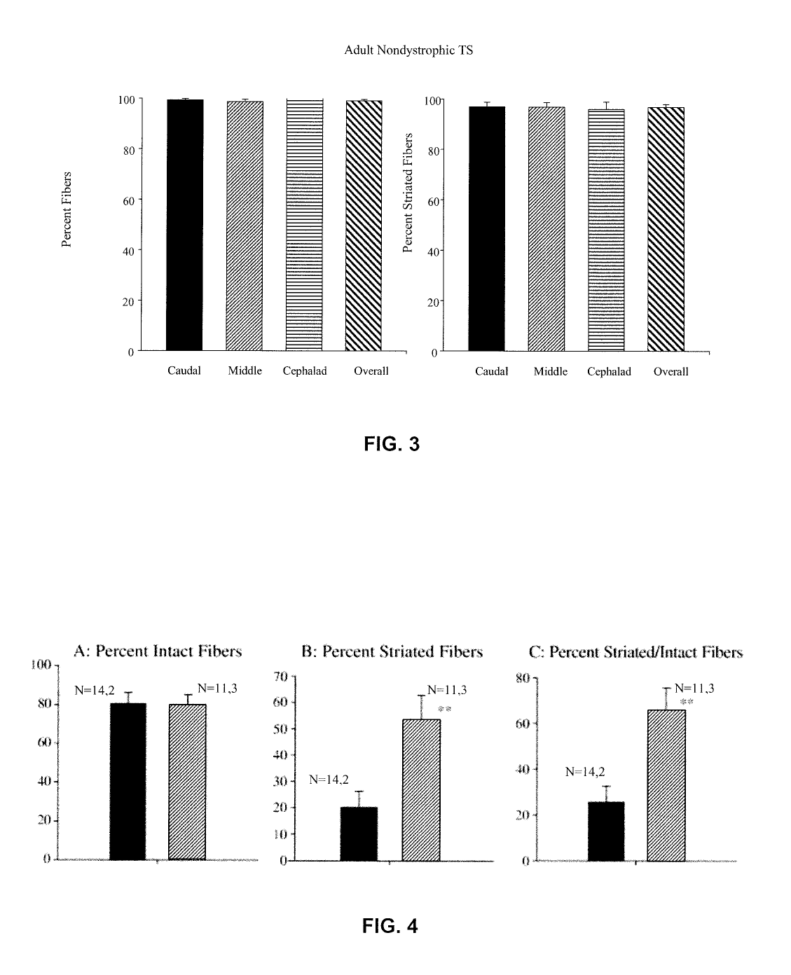

Each preparation may be illuminated using bright-field optics to minimize depth of focus issues in visualizing muscle fiber striations. Since the mdx TS muscle is a flat and thin preparation, all the fibers in each area are roughly within the same focal plane. However, small variations in depth of focus may produce small variations in the appearance of striated fibers. Such effects may be minimized by routinely adjusting the plane of focus to maximize the number of striated fibers in each photographed area. The density of muscle fibers and the density of striated fibers may then be evaluated for each sampled area by drawing a line orthogonal to the principal axis of the TS muscle fibers across the entire viewing area of each photograph. The percentage of the length of this line that covered muscle fibers and the percentage of this line that covered striated muscle fibers (defined as having striations over at least 50% of the observed length) may be used to determine the percentage of muscle fibers and striated muscle fibers, respectively, for each photographed muscle area. Control experiments using these procedures on adult nondystrophic TS muscles (N=37 areas, 2 TS muscles at 19 and 27 months) may yield average values of 99.1±0.5 (SE) percent fibers and 96.7±1.2 percent striated fibers with no regional differences across the caudal, middle, and cephalad thirds of the muscle (FIG. 3).

Cytosolic levels of IκB-α may be determined using Western blot techniques. Cytosolic and nuclear fractions may be obtained from isolated diaphragm muscles using the techniques described in Kumar and Boriek, 2003. Briefly, the muscles may be weighed after removing tendinous components, and frozen and homogenized by mortar and pestle in lysis buffer on ice (1 mg muscle/18 μl lysis buffer containing 10 mM HEPES, 10 mM KCl, 1.5 mM MgCl₂, 0.1 mM EDTA, 0.1 mM EGTA, 1 mM dithiothreitol, 0.5 mM phenylmethylsulfonylfluoride, 2.0 μg/ml leupeptin, 2.0 μg/ml aprotinin, 0.5 mg/ml benzamidine, at pH 7.9). To lyse the cells, the ground tissue may be subjected to two freeze-thaw cycles and subsequently vortexed and centrifuged (13,000 rpm, 10 s). The supernatant cytosolic extract may be immediately frozen (−80° C.) for Western blot analyses, while the nuclear pellet may be resuspended on ice in a nuclear extraction buffer (20 mM HEPES, 420 mM NaCl, 1 mM EDTA, 1 mM EGTA, 25% (v/v) glycerol, 1 mM dithiothreitol, 0.5 mM phenylmethylsulfonylfluoride, 2.0 μg/ml leupeptin, 2.0 μg/ml aprotinin, 0.5 mg/ml benzamidine; at pH 7.9) at a ratio of 4 μl per milligram of muscle weight. The preparation may be incubated on ice with intermittent vortexing before being centrifuged as described above for 5 min. at 4° C. The supernatant nuclear extract may then be frozen (−80° C.) for subsequent biochemical determinations.

Equal amounts of proteins (based on Lowry Assay) from treated vs. untreated cytosolic fractions may be boiled in SDS-PAGE sample buffer for 5 min, applied to 10% SDS-PAGE gel, and blotted onto PVDF (polyvinyl difluoride) membrane. The membrane may be blocked with 5% milk in TBST and immunoprobed with anti-polyclonal IκB-α, #sc-371 (Santa Cruz Biotechnology, Santa Cruz, Calif.), at 1:500 dilution in 5% milk-TBST overnight at 4° C. After three washes, the membrane may be incubated with a 1:10,000 dilution of the appropriate peroxidase-conjugated secondary antibody for 1 h at room temperature. After additional washing steps, the antibody complex may be detected by chemiluminescence using the ECL detection reagent (Amersham) and densitometric measurements may also be obtained. Protein loading levels may be examined by staining the membrane with Coomassie blue dye.

The compositions and methods disclosed here provide a therapy for MD by administering a chemical or biological agent to a subject to modulate the nuclear factor kappa B (NFkappaB or NFκB) pathway such that the activation of NFκB is either inhibited or reduced in the subject's muscle tissues. The phrases “inhibitor of activation” and “NFκB inhibitor” are used interchangeably to refer to an agent that decreases or reduces the transcriptional or other activities attributable to NFκB in the cells.

In one embodiment, an NFκB inhibitor may be an agent that reduces the production of NFκB protein in the cells. Reducing production of NFκB may occur by reduction or inhibitions of transcription of the various subunits of NFkB such as Rel A, P50, and IkB. Reducing production may further occur by reduction or inhibition of translation of the Rel A, P50 and IκB mRNA transcripts into protein. In another embodiment, an NFκB inhibitor may be an agent that stabilizes the IκB protein. In still another embodiment an NFκB inhibitor may increase mRNA and/or protein levels of IκB-α. In another embodiment, an NFκB inhibitor may be an agent that blocks the translocation of NFκB into the nucleus. In another embodiment an NFκB inhibitor may reduce or inhibit transcription and/or translation of IKK. In yet another embodiment, an NFκB inhibitor may be an agent that prevents NFκB from acting as a transcription factor after its translocation into the nucleus.

For purpose of the present disclosure, the term “subject” refers to any animal, including for example, mice, rats, dogs, guinea pigs, rabbits and primates. In the preferred embodiment, the subject is human. While the methods disclosed herein may be used most often in humans, they may also be applied to other animals. The terms “treating” (or “treatment”) means slowing, stopping or reversing the progression of a disorder. In the preferred embodiment, it means reversing the disorder's progression, ideally to a point of elimination. The term “chronic” means any period of time that lasts over 30 days.

An “effective amount” is intended to qualify the amount of active ingredient that will achieve the goal of fewer or less intense symptoms associated with DMD. “Effective” may also refer to improvement in disorder severity or the frequency of incidence over no treatment.

Agents for NFκB inhibition may be chemicals, either inorganic or organic, that show an inhibitory effect on NFκB activation, and combinations thereof. Agents may also include extracts obtained from natural sources, such as those from plants, animals, worms, lower eukaryotes such as fungi, or microorganisms. Agents may also be selected from oligonucleotides, proteins, peptides, or compositions containing antibodies, and combinations thereof.

Agents for NFκB inhibition may be administered orally e.g., as a pill, powder, suspension, syrup, lozenge, spray or gum, or nasally, e.g., as an aerosol spray or mist. Agents for NFκB inhibition may further be administered intravenously or intraperitonialy.

The term “cytokine” refers to proteins released by cells that have a specific effect on the interactions between cells, on communications between cells or on the behavior of cells. Cytokines include interleukins, lymphokines and other cell signaling molecules, such as tumor necrosis factor and the interferons. “NFκB dependent cytokines” means those cytokines whose gene transcription requires activation of NFκB. “Immunoassay” means any assays that utilize an antibody or an antiserum.

The terms “protein,” “polypeptide,” and “peptides” are used interchangeably in this disclosure. A pharmaceutical composition is a mixture containing more than one chemical, or more than one protein. “Inhibit” or “inhibition” means lessening, reducing, attenuating a cellular activity. “Inhibitor” means any agent that is capable of inhibition. The term “substantially” means more than 40%. For example, when the level of a protein is substantially reduced, its level decreases by 40% or more.

Examples of NFκB inhibitors may include carbamates, such as pyrrolidine dithiocarbamate (PDTC), curcumin (diferuloylmethane), and combinations thereof. Other compositions that function to block NFκB activation are also useful. All the references cited in this paper are incorporated by reference to the same extent as though fully replicated herein. By way of examples, these compositions include, but are not limited to:
(1) SN-50, a cell-permeable peptide that inhibits the nuclear translocation of NFκB (D'Acquisto et al., Naunym-Schmiedeberg's Arch. Pharmacol. 364, 422, 2001);
(2) Gabaexate mesilate, a serine protease inhibitor which stabilizes cytosolic IκB-α levels in the presence of tumor necrosis factor (TNF) and decreases the nuclear activation of NFκB (Crit. Care Med., 31(4), 1147, 2001; Yuksel et. al., J. Pharmacol. Exp. ther., 305(1), 2003);
(3) BMS-345541, which stabilizes cytosolic levels of IκB-α by inhibiting IκB kinase (IKK, Burke et al., J. Biol. Chem., 278 (3), 1450-1456, 2003);
(4) The following compounds specifically identified in a review article summarizing several additional agents which inhibit IKK and thereby stabilize or otherwise increase cytosolic levels of IκB-α(Karin et al., 2004—see Table 1):
   (a) The “quinazoline analogue” identified as Compound 1 or SPC-839,
   (b) The “Beta-carbolin analogue” identified as Compound 2 or PS-1145,
   (c) The “amino-thiophenecarboxamide derivative” identified as Compound 4 or SC-514,
   (d) The “ureido-thiophenecarboxamide derivative” identified as

Compound 5,
(e) The “diarylpyridine derivative” identified as Compound 6,
(f) The “anilino-pyrimidine derivative” identified as Compound 7,
(g) The “pyridooxazinone derivative” identified as Compound 8,
(h) The “indolecarboxamide derivative” identified as Compound 9,
(i) The “benzoimidazole carboxamide derivative” identified as Compound 10,
(j) The “pyrazolo(4,3-c) quinoline derivative” identified as Compound 11,
(k.) The “imidazolylquinoline-carboxaldehyde semicarbazide derivative” identified as Compound 12,
(l.) The “amino-imidazolecarboxamide derivative” identified as Compound 13, and
(m.) The “pyridyl cyanoguanidine derivative” identified as Compound 14;
(5) Epigallocatechin-3-gallate and similar polyphenols extracted from Green tea which inhibit the nuclear activation of NFκB by stabilizing cytosolic levels of cytosolic IκB-α (Singh et al., Arthritis and Rheumatism, 46 (8), 2079, 2002);
(6) Diethyldithiocarbamate which inhibits the nuclear activation of NFκB (Xuan Y.-T. et al., Circulation Research, 84, 1095-1109, 1999; Blondeau et al., The J. of Neuroscience, 21(13), 4668, 2001);
(7) The κB decoy DNA sequence identified by Blondeau et al (The J. of

Neuroscience, 21(13), 4668, 2001) which inhibits the nuclear activation of NFκB;
(8) The proteasome inhibitor MG 132 which inhibits the degradation of cytosolic IκB-α and reduces the nuclear activation of NFκB (Takeuchi et al., Digestive Diseases and Sciences, 47(9), 2070, 2002);
(9) The agent diferuloylmethane (curcumin) which is present in yellow curry and which reduces the nuclear activation of NFκB by stabilizing cytosolic IκB-α (Singh and Aggarwal, J. Biol. Chem., 270 (42), 24995, 1995);
(10) The cell-permeable synthetic peptide (NBD peptide; Leu-Asp-Trp-Ser-Trp-Leu) identified by May et al. (Science, 289, 1550, 2000) which inhibits IKK activity and reduces the nuclear activation of NFκB by interfering with specific binding reactions of the IKK complex that are required for IKK activity;
(11) The herbicide 3,4-dichloropropionaniline (propanil) which inhibits nuclear NFκB activation in response to bacterial lipopolysacharides in macrophages (Frost et al., Toxicol. and Applied Pharmacology, 172, 186, 2001);
(12) The water soluble extract of Uncaria tomentosa (cats claw) termed C-Med 100 which inhibits the nuclear activation of NFκB without influencing the stability of cytosolic IκB-α (Akesson et al., Internat. Immunopharmacol., 3, 1889, 2003);
(13) The aqueous extract of Uncaria tomentosa (obtained by boiling cats claw bark for 30 minutes) identified by Sandoval-Chacon et al. (Aliment. Pharmacol. Ther., 12, 1279, 1998) that inhibits the nuclear activation of NFκB;
(14) The hydro-alcoholic extract of Uncaria tomentosa identified by Aguilar et al (J. of Ethnopharmacol., 81, 271, 2002) which inhibits the nuclear activation of NFκB;
(15) Dehydroxymethylepoxyquinomicin (DHMEQ) which inhibits the nuclear translocation of NFκB in cultured Jurkat cells (Ariga et al, J. Biol. Chem., 277 (27), 24625-24630, 2002) and inhibits the nuclear activation of NFκB in obstructed kidneys (Miyajima et al., J. of Urology, 169, 1559-1563, 2003);
(16) Pirfenidone (2(1H)-Pyridinone, 5-methyl-1-phenyl) which inhibits nuclear NFκB activation in cultured hepatocytes exposed to the cytokine IL-1β (Nakanishi et al., J. of Hepatology 41, 730-736, 2004);
(17) The agents, Bay 11-7085 and Bay 11-7082, which inhibit the nuclear activation of NFκB by inhibiting IKK-dependent phosphorylation of the inhibitory protein IκB-α in a variety of cell types (lzban et al., Hum. Pathol., 31(12), 1482-1490, 2000; Mabuchi et al., Clin. Cancer res., 10(22), 7645-7654, 2004; Zou et al., Am. J. Physiol. (Gastrointest Liver Physiol), 284(4), G713-G721, 2003);

(18) Gliotoxin, which decreases the nuclear activation of NFκB by increasing the concentration of cytosolic phosphorylated IκB-α (Pahl et al., J. Exp. Med., 183, 1829-1840, 1996);
(19) The class of sesquiterpene lactones derived from a variety of botanical sources of the Asteracae family including Artemisia annua (Aldieri et al., FEBS Lett, 552, 141-144, 2003), Achillea millefolium (Mustakerova et al., Verlag der Zeitschrift fur Naturforschung, 57c, 568-570), Arnica montana and Arnica chamissonis (Lyβ et al., J. Biol. Chem. 273 (50), 33508-33516), Tanacetum parthenium (“feverfew”; Jan and Kulkarni, J. Ethnopharmacol, 68, 251-259, 1999), Mikania guaco, Milleria quinqueflora, Vanillomopsis arborae, Proteopsis furnensis, Eremanthus mattogrossensis, Tithonia diversifolica (Rungeler et al., Biorganic and Medicinal Chemistry, 7, 2343-2352, 1999), and several species of the genus Carpesium (C. macrocephalum, C. lipskyi, C. Cernuum, C. longfolium; Shi et al., Planta Med., 65, 94-96, 1999; Yang et I., Pharmaxie 56, 825-827, 2001; Yang et al., Planta Med., 68, 626-630, 2002; Yang et al., J. Nat. Prod., 66, 1554-1557, 2003) to include the following specific compounds:
   a.) Parthenolide, which inhibits the nuclear activation of NFκB in Jurkat

T leukemia cells, HeLa cells, mouse L929 fibroblasts, and rat aortic smooth muscle cells by inhibiting the degradation of cytosolic IκB-α (i.e., stabilizing cytosolic IκB-α) in the presence of a variety of agents that stimulate the NFκB pathway (Hehner et al., J. Biol. Chem., 273(3), 1288-1297, 1998; Wong and Menendez, Biochem. Biophysica Res. Comm., 262, 375-380, 1999). It has also been shown to inhibit IKK activity and the nuclear activation of NFκB in HeLa cells (Kwok et al., Chem and Biol., 8, 759-766, 2001) and to inhibit the binding of NFκB to the κB consensus sequence in nuclear extracts of rat lungs (Sheehan et al., Molec. Pharmacol., 61, 953-963, 2002).
b.) Artemisinin, which blocks the nuclear activation of NFκB in cultured human astrocytoma T67 cells exposed to a mix of lipopolysacharrides and cytokines (Aldieri et al., FEBS Letters, 552, 141-144, 2003).
c.) Helenalin, which inhibits nuclear activation of NFκB by TNFα in Jurkat T cells by alkylating the p65 subunit and inhibiting binding to the κB consensus sequence (Lyβ et al., J. Biol. Chem., 273, 33508-33516, 1998; cf Rungeler et al., Biorganic and Medicinal Chemistry, 7, 2343-2352, 1999).
d.) Mexicanin I, which inhibits the nuclear activation of NFκB in TNFα stimulated Jurkat T cells (Lyβ et al., J. Biol. Chem., 273, 33508-33516, 1998).
e.) 2,3-Dihydroaromaticin, which inhibits the nuclear activation of NFκB in TNFα stimulated Jurkat T cells (Lyβ et al., J. Biol. Chem., 273, 33508-33516, 1998).
f.) Helenalin-isobutyrate, which inhibits the nuclear activation of NFκB in TNFα stimulated Jurkat T cells (Lyβ et al., J. Biol. Chem., 273, 33508-33516, 1998).
g.) Isohelenin, which stabilizes cytosolic IκB-α and inhibits the nuclear activation of NFκB in cultured rat aortic smooth muscle cells (Wong and Menendez, Biochem. Biophysica Res. Comm., 262, 375-380, 1999).
(20) Arctigenin and related dibenzylbutyrolactone lignans such as demethyltraxillagenin, which inhibit the nuclear localization of NFκB by stabilizing cytosolic IκB-α in Raw 264.7 mouse macrophages (Cho et al., International Immunopharmacology, 2, 105-116, 2002);
(21) Sulfasalazine, which inhibits the nuclear activation of NFκB in cultured human colonic epithelial cells (Wahl et al., J. Clin. Invest., 101, 1163-1174, 1998), in human colonic biopsies from individuals treated with chronic oral doses of the drug (Gan et al., J. of Gastroenterology and Hepatology, 20, 1016-1024, 2005), in human adipose tissue and skeletal muscle explants from biopsies obtained from healthy pregnant women (Lappas et al., Endocrinology, 146 (3), 1491-1497, 2005), in human glioblastomas (Robe et al., Clinical Cancer Research, 10, 5595-5603, 2004), and in Jurkat T cells (Weber et al., Gastroenterology, 119, 1209-1218, 2000) by a mechanism involving stabilization of cytosolic IκB-α (Wahl et al., 1998) and inhibition of IKK activity (Weber et al., 2000);
(22) Guggelsterone, which inhibits the nuclear activation of NFκB by inhibiting IKK in a variety of cancer cells in which enhanced nuclear NFκB is either constitutive or secondary to external activation of the NFκB pathway by a variety of agents (Shishodia and Aggarwal, J. Biol. Chem., 279 (45, November 5), 47148-47158, 2004);
(23) Troglitazone, which inhibits the nuclear activation of NFκB in mononuclear leucocytes by reducing total NFκB levels and increasing total IκB-α levels in obese humans treated with daily oral doses of the drug (Ghanim et al., J. of Clin. Endocrinology and Metabolism, 86(3), 1306-1312, 2001);
(24) The methanol extract of the plant Saururus chinensis identified by Kim et al. (Biol. Pharm. Bull., 26(4),481-486,2003) which inhibits the nuclear activation of NFκB by stabilizing cytosolic IκB-α in RAW 264.7 mouse macrophages;
(25) N-acetylcysteine, which reduces NFκB nuclear activation that is induced by hypoxia in mouse embryonic fibroblasts by specifically inhibiting NFκB binding to DNA and thereby inhibiting hypoxia-induced increases in the anti-apoptotic gene product, XIAP (Qanungo et al., J. Biol. Chem., 279(48), 50455-50464, 2004);
(26) Phenylmethyl benzoquinone derivatives, which are shown to inhibit the production of inflammatory mediators and the activation of NF-κB (U.S. Pat. No. 6,943,196);
(27) Xanthine derivatives, which are shown to inhibit NF-κB activation (Japanese Unexamined Patent Publication (kokai) No, 9-227561);
(28) Isoquinoline derivatives, which are shown to inhibit NF-κB activation (Japanese Unexamined Patent Publication (Kokai) No. 10-87491);
(29) Indan derivatives, which are shown to inhibit NF-κB activation (U.S. Pat. No. 6,734,180);
(30) Alkaloids originated from a plant belonging to the genus Stephania of the family Menspermaceae, and their derivatives and salts thereof, which are shown to inhibit NF-κB activation (U.S. Pat. No. 6,123,943);
(31) Agents that inhibit NF-κB activation by modulating the ubiquitin degradation pathway, such as peptides that resemble the recognition domain for E3 ubiquitin ligase (See, e.g., U.S. Pat. No. 6,656,713 and U.S. Pat. No. 5,932,425); and
(32) Antisense oligonucleotide which hybridizes to NFκB mRNA and thus inhibits NFκB dependent pathways (U.S. Pat. No. 6,498,147).

Combinations of these agents or combinatorial use of more than one agent are particularly preferred and may have significant therapeutic advantages in the treatment of MD. In one aspect, the toxicity or side effects of individual compositions may be reduced or practically eliminated when using lower dosages of individual compositions to achieve the same or better therapeutic efficacy as may be obtained from a larger dose of any one composition. Furthermore, the compositions may block the pathway at multiple points to achieve greater therapeutic effects.

An additional benefit of using these compositions is that of improving various qualities of muscular quality. One class of improvement is that of morphology of dystrophic skeletal muscles, particularly in dystrophic muscles that are exposed to chronic passive stretch. The compositions particularly improve the sarcomeric organization of dystrophic muscles and increase the survival of striated muscle fibers in dystrophic muscles by opposing those pathogenic mechanisms responsible for the streaming of Z lines in dystrophic muscle (Cullen, M. J., Fulthorpe, J. J., 1975. Stages in fibre breakdown in Duchenne Muscular Dystrophy: An electron-microscopic study. J of the Neurol. Sci. 24, 179-200.). The compositions may also improve muscle fiber cross sectional diameter, increase the number of muscle nuclei per fiber cross section, and reduce the percentage of centrally located nuclei. A second class of functional improvement is in resting membrane potential, particularly in dystrophic muscles that are exposed to passive muscle stretch. Additionally, there is improvement in whole body strength, as determined by measuring the total body strength exerted by mice exhibiting muscular dystrophy as initially described in Carlson et al.(Muscle and Nerve, 13:480-84, 1990) and an improvement in the development of tension in the limb musculature.

Compositions disclosed herein may be administered alone or in combination with, for example, other NFκB inhibitors, steroids, anesthetics, antiepileptics, other agents that affect gene expression, or combinations thereof.

Compositions disclosed herein may be administered, for example, peritonealy, to a subject diagnosed with dystrophin deficiency or muscular dystrophy, either by intermittent or continuous intravenous administration or by injection in the muscles. Administration can be given either through a single dose or a series of divided doses. Compounds in various formulations of pharmaceutically effective amounts for treating MD may be used in combination or sequentially and may be administered by intermittent or continuous administration via implantation of a biocompatible, biodegradable polymeric matrix delivery system, via a subdural pump inserted to administer compounds directly, or by intranasal, oral, or rectal administration.

It is desirable to monitor the physiological and morphological changes before and after administration of the compositions disclosed herein. The expression levels of NFκB and IκB in the cells may be measured at both the mRNA and protein levels by Northern blot and Western blot analysis, flow cytometry or ELISA. The activation of NFκB may be measured using the Trans AM assays or other methodology known to artisans in the field.

Blood or tissue samples may be taken from subjects treated with the disclosed compositions to measure the expression profiles of various cytokines as a result of the treatment. Gene expression profile may be analyzed by microarray, RT-PCR, Northern blot, Western blot or by ELISA analyses. Preferably, the levels of TNFα, IL-6 and IL-1β are periodically monitored to assess treatment efficacy.

Changes or modifications may be made in the methods and systems described herein without departing from the spirit hereof. It should thus be noted that the matter contained in the above description or shown in the accompanying figures and examples should be interpreted as illustrative and not in a limiting sense.

Various references, including patents, patent applications and other scientific literatures are cited throughout this application. Full citations for the scientific literatures can be found at the end of the Specification immediately proceeding the Claims. All references cited in this disclosure, including papers, patents, patent applications, meeting abstract, etc, are expressly incorporated by reference into this application.

### Example 1

### PDTC May Stabilize Cytosolic IκB-α in Adult mdx Skeletal Muscle

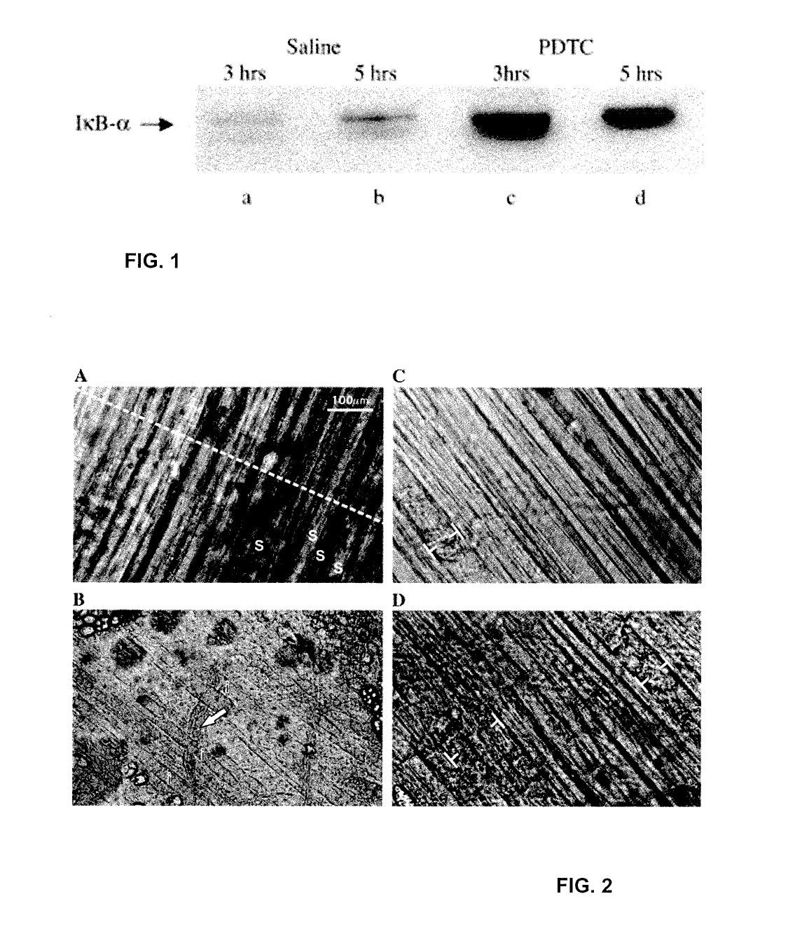

Previous studies have shown that PDTC reduces nuclear NFκB activation (Cuzzocrea et al., 2002, D'Acquisto et al., 1999, D′Acquisto et al., 2001, Rangan et al., 1999, Satoh et al., 1999 and Takeuchi et al., 2002) by stabilizing cytosolic IκB-α (Cuzzocrea et al., 2002) in various tissues from mice and rats. To determine if the doses used in the present study have similar mechanisms of action in adult mdx skeletal muscle, mdx mice were administered either a single ip dose of 50 mg/kg PDTC or vehicle (HEPES Ringer) prior to euthanization and isolation of the diaphragm muscle. Western blots of cytosolic IκB-α obtained at 3 and 5 h after vehicle injection indicated ambient levels of the inhibitory protein in freshly isolated diaphragm muscle (FIGS. 1a and b). A single injection of PDTC substantially increased ambient levels of cytosolic IκB-α at corresponding time points in littermate diaphragms (FIGS. 1c and d). These results indicate that a single dose of PDTC may substantially increase ambient cytosolic IκB-α levels in adult mdx skeletal muscle for a period of at least 5 h.

### Example 2

### Chronic PDTC Administration May Reduce the Loss of Striated Fibers and Have Beneficial Effects on the Structure of mdx TS Muscle

Previous studies indicated a significant loss of muscle fibers in the adult mdx TS that did not occur in the nondystrophic TS and that was characterized by an overall 45% reduction in the thickness of the TS based upon the number of fiber layers seen in cross section (Carlson et al., 2003). In contrast to nondystrophic TS fibers which were uniformly striated (FIG. 3), surviving adult mdx TS fibers exhibited discrete cytoplasmic areas devoid of myofibrillar material, areas of hypercontraction, large areas of cytoplasmic rarefaction with delta lesions, and areas of apparent myofibrillar degeneration. By about 2 years of age, the mdx TS appeared as a thin (about 50-100 μm thick) fibrous layer with only a few muscle fibers that lacked myofibrillar organization. Large areas devoid of muscle fibers were characterized by extensive fibrosis with collagen fibrils and numerous fat cells at least as early as 5 months and progressing throughout the life of the mouse (Carlson et al., 2003).

In freshly isolated preparations of mdx TS muscle, hypercontraction and myofibrillar disorganization are expressed as a decrease in the appearance of striated muscle fibers (See e.g., FIG. 2A) and fiber loss and fibrosis appear as a decrease in the number of fibers in individual areas of the TS (See e.g., FIG. 2B; cf. Carlson et al., 2003 and Cullen and Fulthorpe, 1975). In order to quantify these observations and rapidly assess large areas of the mdx TS muscle, several photomicrographs were obtained over large portions of each TS muscle and the proportion of each microscopic field exhibiting muscle fibers and striated muscle fibers was determined for each area sampled. Nondystrophic muscles examined using this technique exhibited uniform levels of muscle fibers and uniform levels of striated muscle fibers throughout the TS muscle (FIG. 3).

The effect of aging on untreated mdx TS muscle is apparent by comparing FIGS. 2A and B, which represent the middle region of the TS muscle at 9 months and 15 months, respectively. Although the untreated 9-month mdx TS muscle exhibited muscle fibers across the entire photographed area, only a small percentage (22%) of these fibers were striated (FIG. 2A). In contrast, the 15-month preparation exhibited only a few muscle fibers (<10%), and no striated fibers at the same magnification over the middle region of the TS muscle (FIG. 2B). This comparison demonstrates the fiber loss that normally occurs in the mdx TS muscle and emphasizes the experimental utility of this particular preparation in directly assessing the damaging effects of chronic passive stretch on dystrophic muscle fibers.

In the first series of PDTC experiments, mdx mice aged 8.5-9 months at sacrifice received daily injections of PDTC at doses between 50 and 75 mg/kg for a period of 27-30 consecutive days. FIG. 2C shows the effects of this treatment on the middle region of the TS muscle. In contrast to the area from an untreated mouse (FIG. 2A), the PDTC-treated mdx TS exhibited approximately 82% striated fibers (FIG. 2C). The combined results from this first series of experiments indicated that both untreated and PDTC-treated preparations exhibited an average percent fibers of about 80%, but that the PDTC-treated preparations had a highly significant (P<0.01) 3-fold increase in the percentage of striated muscle fibers (FIGS. 4A, 4B, 4C). These results prompted additional studies using older mdx mice.

In the second series of experiments (Series 2 investigations), mdx mice aged 9.5-16 months at the beginning of the experiment and 11.5-18.5 months at sacrifice (average age at sacrifice for all saline and PDTC-treated mice in this series was 14.8 months) were treated with daily injections of 50 mg/kg PDTC. In this and subsequent experiments, the percentage of muscle fibers and of striated muscle fibers were evaluated in 3 regions of the TS; the caudal third extending towards the xiphoid process, the middle third, and the cephalad third of the muscle. Control experiments using these procedures on adult nondystrophic TS muscles (N=37 areas, 2 TS muscles at 19 and 27 months) yielded average values of 99.1±0.5 (SE) percent fibers and 96.7±1.2 percent striated fibers with no regional differences across the caudal, middle and cephalad thirds of the muscle (FIG. 3). A third series of experiments using daily 50 mg/kg PDTC injections (Series 3 investigations) was conducted using aged mdx mice which were 19-20 months old at the beginning of the experiment and 21.5-22 months old at sacrifice (Series 3). Littermate or age-matched saline-injected mdx mice served as controls.

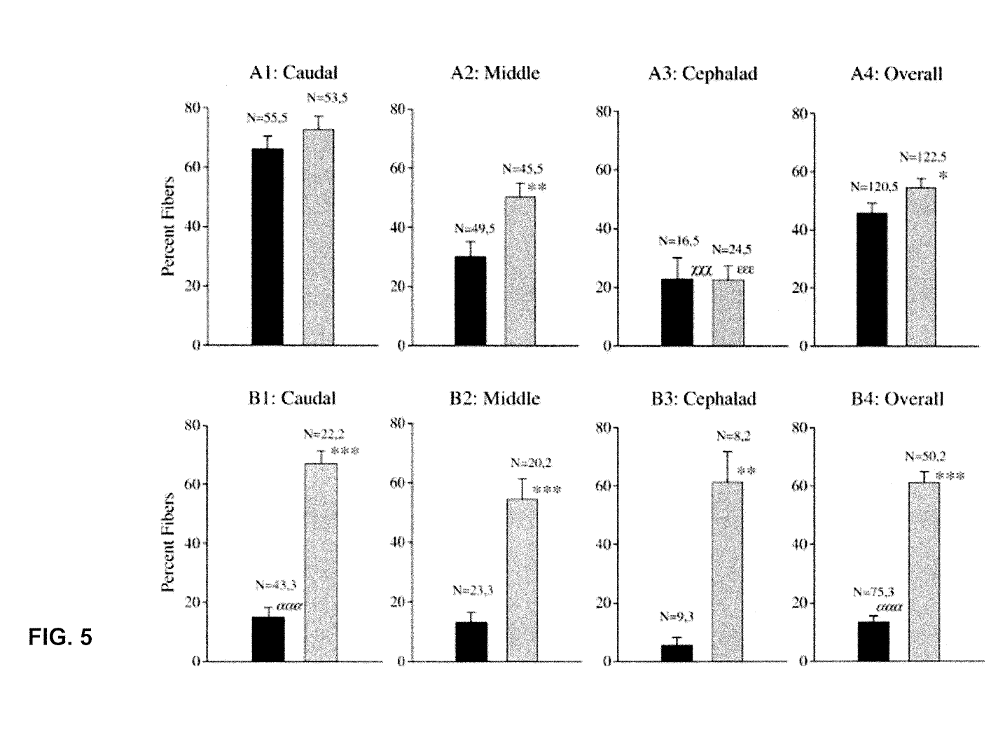

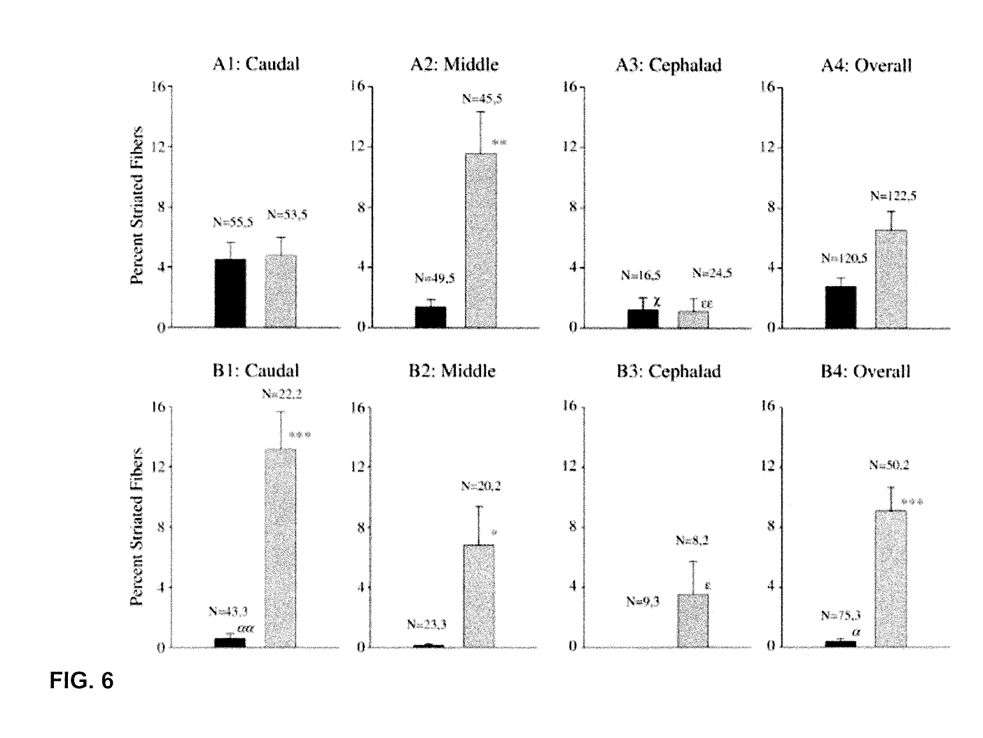

The Series 2 TS muscles exhibited a significant (P<0.001) effect of TS region on the percentage of fibers in both the saline-injected and PDTC-treated preparations (FIG. 5A1, 5A2, 5A3, 5A4). In each case, the percent of fibers declined progressively in the cephalad direction. PDTC treatment produced a significant (P<0.01) 1.7-fold increase in the percent of fibers in the middle (compare FIGS. 2B and 2D) third of the muscle (FIG. 5A2), and a significant increase in the overall percent of fibers across all regions of the muscle (overall; P<0.05). A significant effect of region on the percent of striated fibers was also observed for the Series 2 saline-injected mice at an average age of 14.6 months (P<0.05; FIGS. 6A1, A2, A3). The PDTC-treated mice at this age exhibited a large (8.4-fold) and significantly (P<0.01) increased percent of striated fibers in the middle region (compare FIGS. 2B and 2D) of the TS muscle (FIG. 6A2).

There was a significant effect of age (between Series 2 and Series 3) on the percent of fibers (FIG. 5B1, 5B2, 53, 5B4) and striated fibers (FIG. 6B1, 6B2, 6B3, 6B4) in the saline-injected mice; and at 22 months, an average of less than 20% of each sampled area contained fibers (black bars in FIGS. 5B1-B4). The decline in fibers with age was highly significant (P<0.001) in the caudal region (compare FIGS. 5A1-B1) and in the overall total of all areas sampled across the TS (compare FIGS. 5B4-A4) but did not reach statistical significance in the middle or cephalad regions. At 22 months, the percent of striated fibers was less than 1% in all three TS regions of the saline-injected mice (FIGS. 6B1-B3; black bars) and was significantly decreased compared to Series 2 in the caudal region (FIG. 6B1; P<0.01) and in the overall total of all sampled areas (FIG. 6B4; α, P<0.05). At 22 months, no significant regional effect was observed for either the percent of fibers (FIG. 5B1-5B4) or striated fibers (FIG. 6B1-6B4) in the saline-injected (vehicle) mice. These results indicate that fiber loss and loss of striations in the mdx TS initially occur in the cephalad and middle regions and proceed caudally with aging (see FIG. 5 and FIG. 6).

PDTC treatment of the aged mdx mice (22 months) produced large (4.1- to 11.1-fold) and significant increases in the density of muscle fibers across all regions (FIGS. 5B1-B4; P<0.01 or P<0.001) and substantially increased (22- to 68-fold) the percent of striated fibers in the caudal (FIG. 6B1; P<0.001), middle (FIG. 6B2; P<0.05), and overall total of all sampled areas (FIG. 6B4; P<0.001). The PDTC-treated mice at this age did not exhibit a significant effect of region on the percent of fibers (FIGS. 5B1-B3) but did exhibit a significant effect of region (P<0.05) on the percent of striated fibers, with a progressive decline in this value proceeding in the cephalad direction (FIGS. 6B1-B3).

A fourth series of experiments was conducted using a longer period of PDTC administration beginning in younger 5 month mdx mice. The results of these experiments were similar to those in Series 1 through 3 and indicated a significant effect (P<0.001) of PDTC treatment on the percent of striated fibers in both middle and cephalad regions. In particular, treatment of an individual mdx mouse with injections (50 mg/kg) in 238 out of a total of 258 days resulted in an overall percent striated fibers of 40.4±2.6 (N=25 areas) and an overall percent fibers of 82.2±2.6 (N=25) at 13.5 months of age. Since saline-injected mice at an average age of 14.6 months exhibited an overall percent of striated fibers of approximately 2.8±0.6% (FIG. 6A4) and an overall percent fibers of 45.7±3.4%, this result provides strong evidence that PDTC treatment at earlier ages may at least partially prevent the loss of striated fibers in chronically stretched dystrophic muscle. Overall, these results indicate that PDTC treatment reduced the loss of striated muscle fibers by inhibiting and partially reversing a pathogenic mechanism that normally proceeds in the cephalad to caudal direction in the untreated mdx TS muscle.

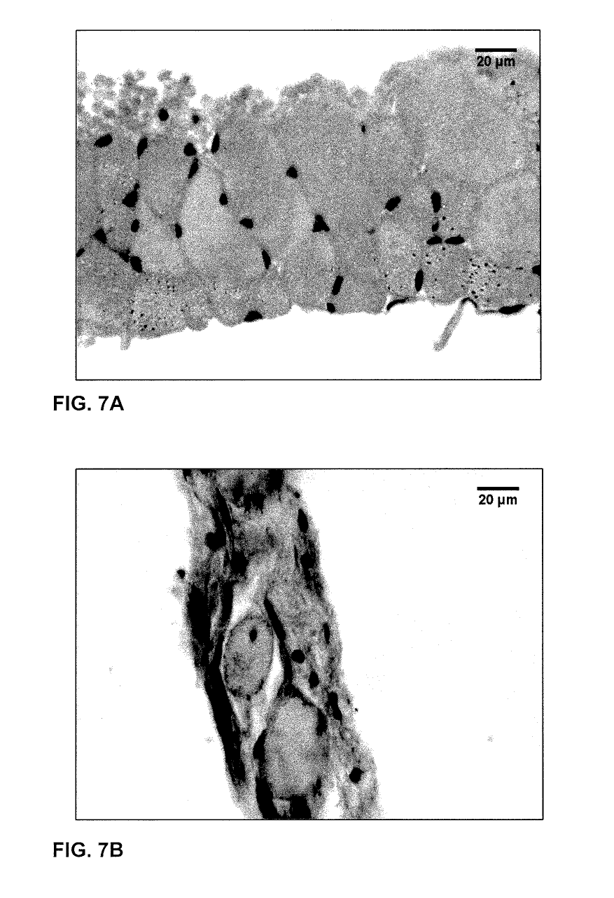

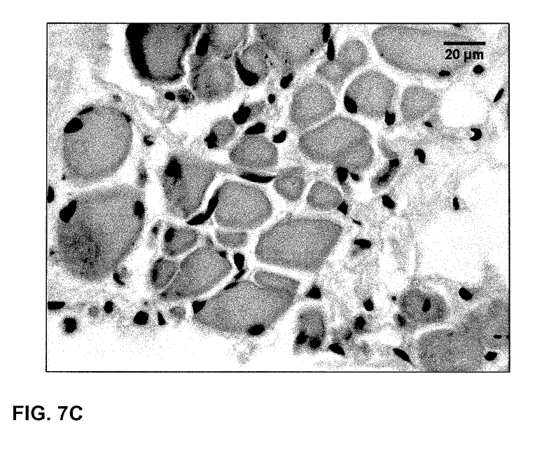

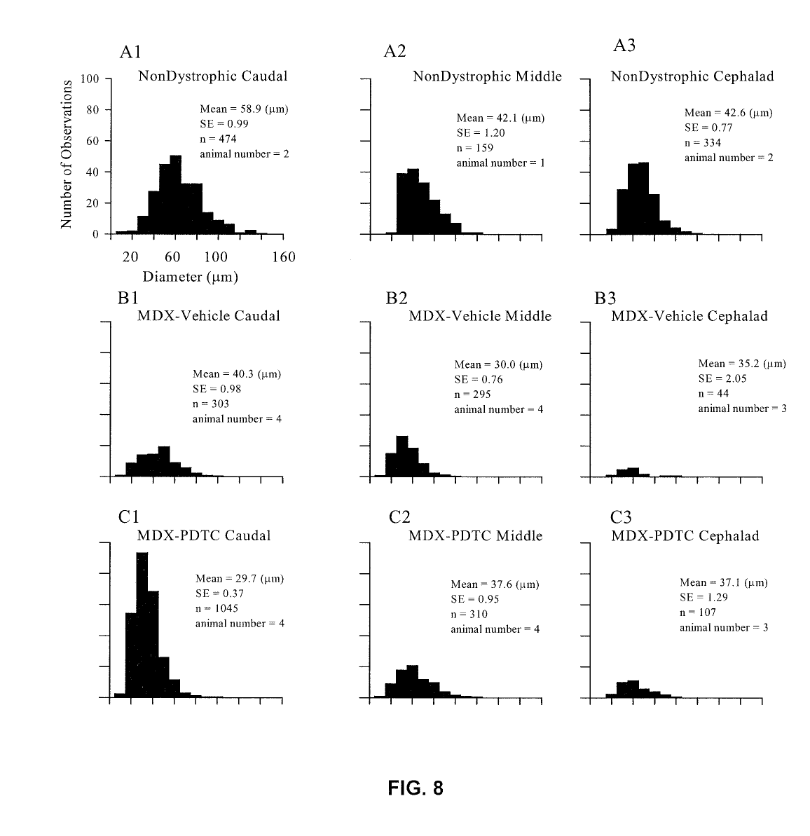

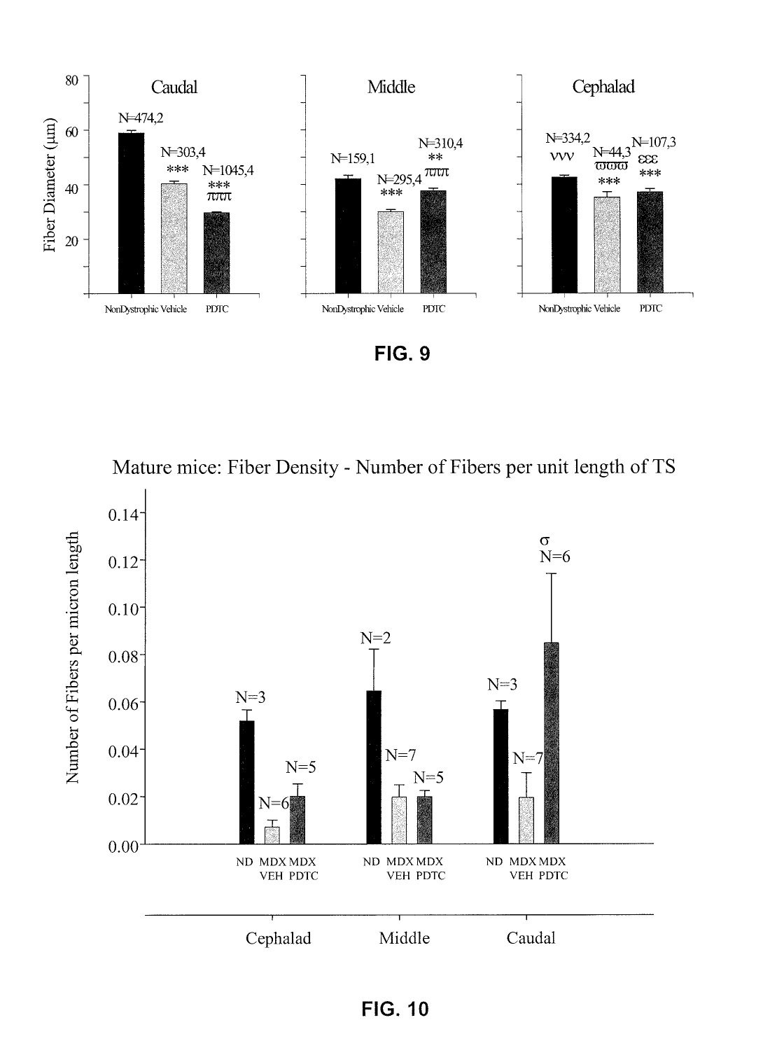

To further examine the morphological effects of chronic treatment with inhibitors of the NFκB pathway, the fixed TS preparations used in these investigations on mature mdx mice (Series 2 through 4) were sectioned and stained with hematoxylin and eosin using standard histological procedures (FIG. 7A, 7B, 7C). The results indicate that there is a total of approximately 500 to 600 fibers in the entire nondystrophic TS (FIG. 8A1, 8A2, A3). Vehicle treated mdx TS muscles from the Series 2 and 3 PDTC experiments exhibited a 68% reduction in fibers in the caudal region, a 54% reduction in the middle region, and a 91% reduction in the cephalad region (FIG. 8B1, 8B2, 8B3). Results from age-matched PDTC-treated mice indicated substantial increases in fiber number in the caudal region (FIG. 8C1), no change in the middle region (FIG. 8C2), and an increase in fiber number in the cephalad region (FIG. 8C3). All TS preparations (nondystrophic, mdx vehicle, and mdx PDTC treated) exhibited a significant (p<0.001) effect of region on fiber diameter (FIG. 9), a result which suggests that different magnitudes of passive stretch may differentially influence signaling pathways controlling fiber hypertrophy. Vehicle-injected mdx TS fibers exhibited a significant (p<0.001) reduction in diameter in all regions of the TS muscle in comparison to nondystrophic preparations (FIG. 9). PDTC treatment produced a significant (p<0.001) reduction in diameter in the caudal region and a significant (p<0.001) increase in diameter in the middle region in comparison to corresponding values from the vehicle-injected mdx mice (FIG. 9).

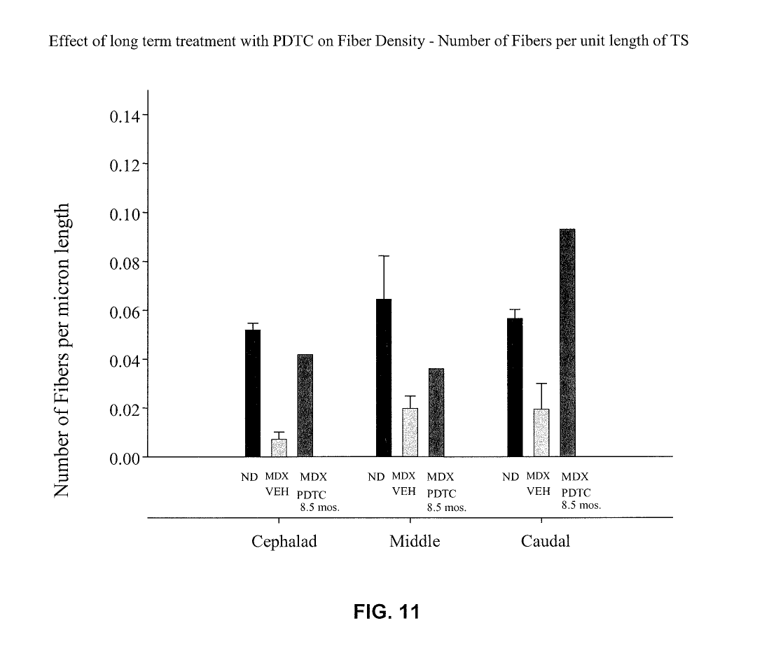

To determine the density of muscle fibers in the mdx TS muscle, individual sections representing all of the fibers within each TS muscle were evaluated by determining the average number of fiber cross sections per unit length of the TS muscle where the length is defined along the axis orthogonal to the principal fiber axis. This procedure was performed for all of the available TS muscles initially used in the Series 2 and 3 PDTC investigations. Mdx TS muscles treated with vehicle exhibited substantial decreases in fiber density in comparison to nondystrophic muscle and treatment with PDTC increased the number of fibers per unit length in both the cephalad and caudal regions (p<0.05) of the mdx TS muscle (FIG. 10). In particular, treatment of an individual mdx mouse with injections (50 mg/kg) in 238 out of a total of 258 days beginning at an age of 5 months and ending at 13.5 months of age resulted in substantial improvements in the density of fibers in comparison to 14.6 month vehicle treated mdx mice (FIG. 11).

These results indicate that treatment of mature mdx mice with PDTC increases fiber number and density (FIGS. 8,10) and reduces fiber diameter (FIG. 9) in the caudal TS region. Chronic PDTC treatment increased fiber diameter (FIG. 9) without altering fiber number or density (FIGS. 8 and 10) in the middle region of the TS. Each of these effects increases the total cross sectional diameter of working skeletal muscle and is therefore beneficial for dystrophic muscle structure and function. However, the differential effects of the chronic PDTC treatment on fiber diameter and fiber number may have important implications regarding satellite cell proliferation and fusion in dystrophic muscle. One explanation is that the signaling environment in the caudal dystrophic TS exposed to NFκB inhibitors favors the continued fusion of satellite cells into newly regenerated fibers leading to fiber splitting, increased numbers of fiber cross sections, and reduced fiber cross sectional diameters; while the environment in the middle TS favors fusion into a few relatively mature fibers and subsequent activation of pathways promoting differentiation and hypertrophy. These results indicate a therapeutic benefit of chronically inhibiting the NFκB pathway that may be facilitated by adjunctive treatment with inhibitors of other cell signaling pathways in order to compensate for differences in cell signaling which may depend upon the different functions of various dystrophic skeletal muscles.

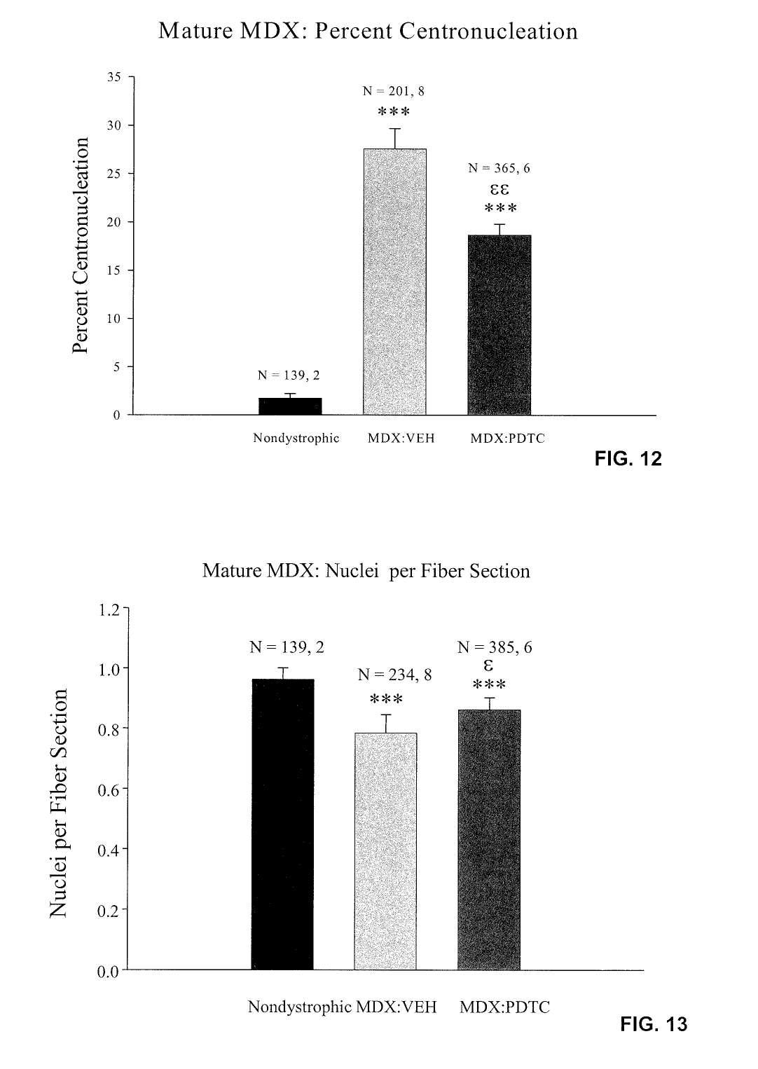

Determination of the total number of myonuclei across all regions of the mature nondystrophic, mdx-vehicle, and mdx-PDTC treated TS muscles in conjunction with determination of the total number of fiber cross sections in the same preparations provided an immediate measure of the number of myonuclei per sectioned fiber (FIG. 12). The results from the Series 2 and 3 PDTC investigations on mature mdx mice indicated that vehicle-injected mdx preparations exhibited a significant (p<0.001) decrease in the number of myonuclei per fiber and that PDTC treatment resulted in a significant (p<0.001) increase in the number of myonuclei per fiber in comparison to age matched vehicle treated mice. Percent centronucleation was increased (p<0.001) in the mdx-vehicle treated TS muscles in comparison to corresponding nondystrophic muscles, and PDTC treatment significantly (p<0.001) reduced percent centronucleation in mature mdx mice (FIG. 13; Series 2 and 3 investigations).

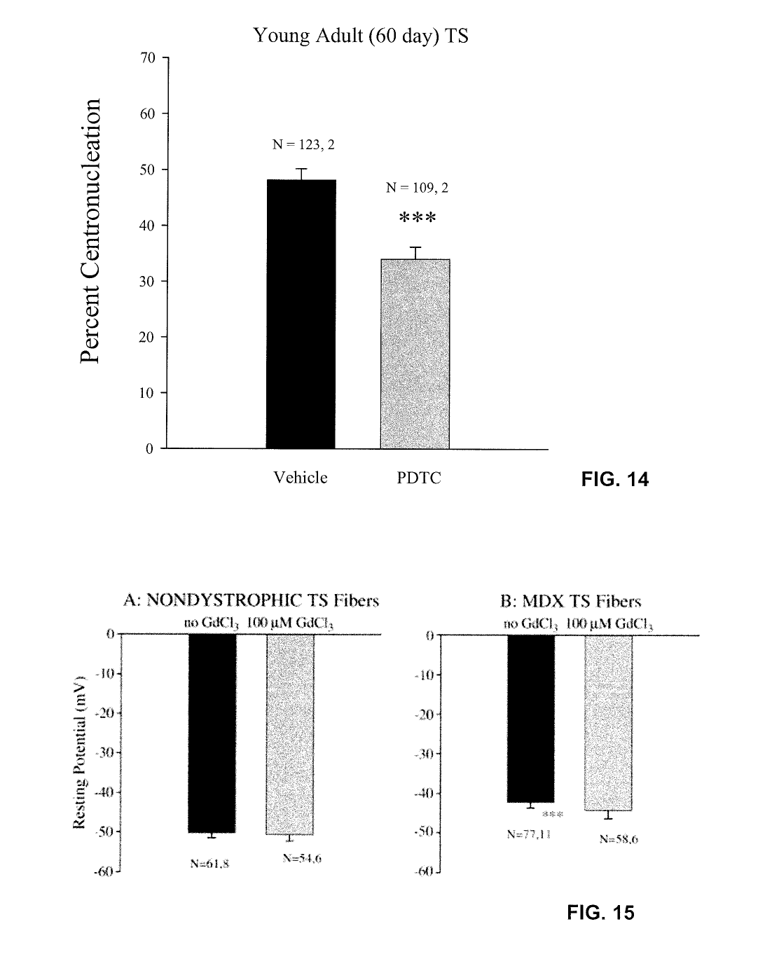

Another demonstration of the beneficial effects of PDTC treatment in reducing the percent centronucleation in dystrophic muscle was obtained by treating 30 day old mdx mice with daily injections of either vehicle or with PDTC (50 mg/kg) for a period of 30 days, and then determining the percent centronucleation at 60 days of age. PDTC treatment for a period of 30 days significantly (p<0.001) reduced the percent centronucleation observed in the young adult (60 day) mdx TS muscles (FIG. 14). These morphological results provide direct evidence that inhibitors of the NFκB pathway may have beneficial effects in treating both young and mature dystrophic preparations.

### Example 3

### Mdx TS Muscle Fibers Exhibit Reduced Resting Membrane Potentials That are Not Secondary to Enhanced Divalent Cation Influx

The effect of the Ca²⁺ channel blocker, Gd³⁺, on the resting membrane potential was examined in both nondystrophic and mdx TS muscle preparations. Gd³⁺ blocks both nonselective cation channels and more Ca²⁺-selective leak channels (Franco et al., 1991 and Yang and Sachs, 1989) and, at concentrations of 20-100 μM, eliminates fluorometric determinations of resting Ca²⁺ influx in a variety of cells (Broad et al., 1999, Carlson and Geisbuhler, 2003, Cox et al., 2002 and Samadi et al., 2005). Depending upon the contribution of resting Ca²⁺ influx to the resting membrane potential, addition of blocking concentrations of Gd³⁺ would be expected to hyperpolarize the plasma membrane, and an elevated resting Ca²⁺ influx in mdx muscles would be characterized by an enhanced sensitivity to the hyperpolarizing influence of 100 μM GdCl₃.

The potential effect of Gd³⁺ on resting potential was determined in several nondystrophic (5-17 months) and mdx (5-11.5 months) TS preparations by first recording from several cells in normal HEPES Ringer solution, and then adding 100 μM GdCl₃ to the solution while recording continuously from a single fiber. After removing the electrode from the fiber, the resting potential of several additional fibers were determined in the presence of the lanthanide. The average resting potential in nondystrophic fibers bathed in normal HEPES Ringer was −50.1±1.4 (SE) mV, a value lower than that recorded in this laboratory from nondystrophic diaphragm (−63±1.3 mV, N=98 fibers, 5 weeks to 2 years of age; Carlson and Roshek, 2001). In agreement with previous results from the diaphragm (Carlson and Roshek, 2001), however, mdx TS fibers had a significantly (P<0.001) reduced resting potential of −42.3±1.4 mV (FIG. 15A, 15B).

No effect on resting potential was observed when Gd³⁺ was added while recording from either nondystrophic or mdx fibers. The results from the total sample of fibers indicated a slight hyperpolarization in the presence of Gd³⁺ that failed to reach statistical significance in either the nondystrophic or mdx TS preparations (see FIG. 15). These results are consistent with previous fluorometric studies indicating no significant differences between resting Ca²⁺ influx in nondystrophic and mdx TS muscle fibers (Carlson et al., 2003) and further demonstrate that resting Ca²⁺ influx is not likely to be responsible for the significant resting depolarization that is characteristic of dystrophic muscle fibers (Carlson and Roshek, 2001, Nagel et al., 1990 and Sakakibara et al., 1977).

### Example 4

### Chronic PDTC Treatment May Restore or Substantially Improve Resting Membrane Potential in mdx TS Fibers

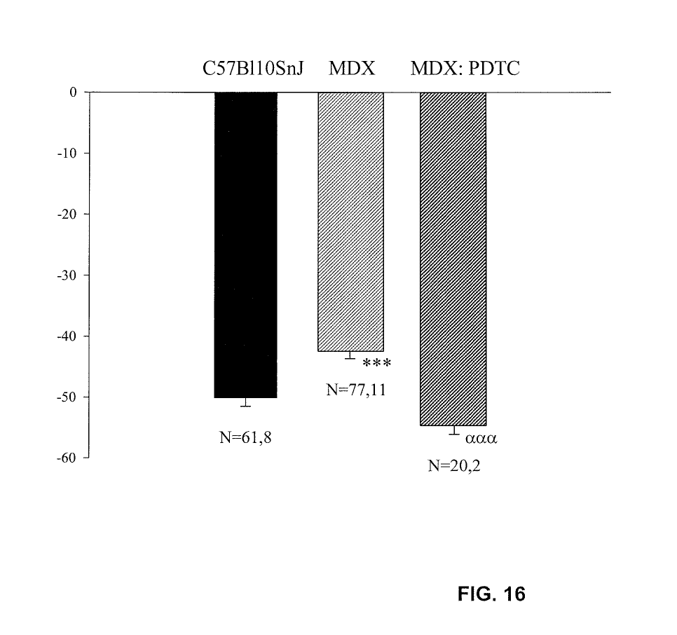

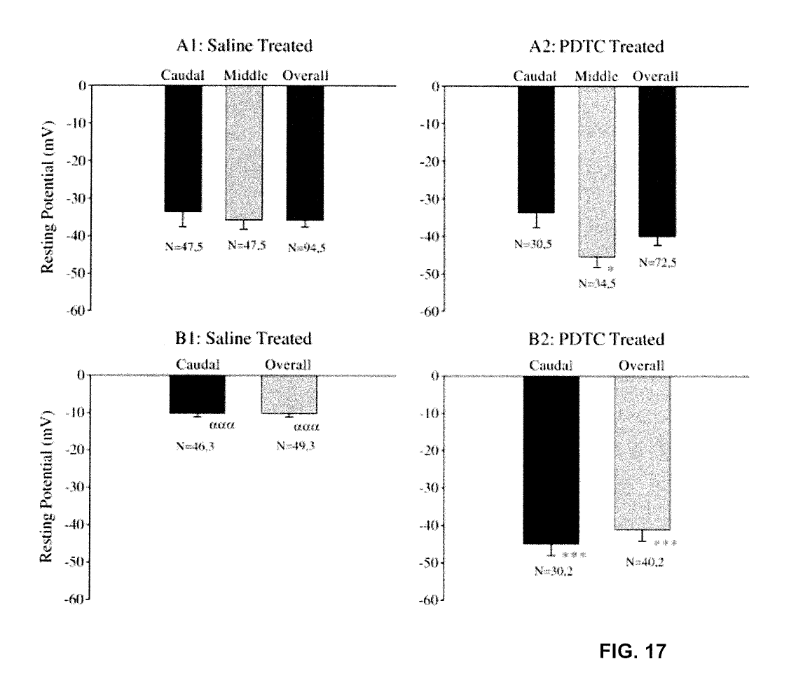

In the Series 1 experiments, daily PDTC treatment beginning at 6.5-7 months completely restored the resting potential to the levels seen in nondystrophic preparations within the middle and caudal regions of the TS (FIG. 16). The mature saline-injected mdx mice used in the Series 2 experiment (12-18 months at sacrifice; average age 14.6 months) exhibited lower resting potentials (overall average=−35.9 mV±1.8 SE) than the younger group of untreated mdx mice used in the initial Gd³⁺ investigations (5-11.5 months; average age 8.2 months). Daily treatment with PDTC increased the fiber resting potential for the older Series 2 mdx mice (FIG. 17A, Series 2) with a significant (P<0.05) increase in the resting potential in the middle region (from −35.8 to −45.4 mV) but not in the caudal region (FIGS. 17A1 and A2).

A significant decrease in resting potential was observed in the aged sample (Series 3) of saline-injected mice (22 months at sacrifice) in comparison to the Series 2 mice (14.6 month average age; FIG. 17B1; P<0.001), with the average resting potential declining to approximately −10 mV. At this age, there were not a sufficient number of intact fibers in either the middle or cephalad regions of saline-injected mice to obtain statistically meaningful determinations from these regions. However, following PDTC treatment it was possible to record from fibers in the middle region of the aged mdx mice (Series 3), where a substantially larger average resting potential of −32.2±6.5 mV (N=9 fibers, 2 mice) was observed (data not shown in FIG. 17). The PDTC-treated mice at this age also showed a highly significant increase in resting potential to approximately −45 mV in the caudal region and −40 mV in the combined data from middle, caudal, and cephalad regions (“overall”) in comparison to their saline-injected littermates (FIG. 17B2; P<0.001).

In the fourth series of PDTC experiments in which chronic administration of the drug was initiated in younger mdx mice (5 months), the overall resting potential obtained at 6.5-13.5 months (49.3±1.4 mV; N=64 fibers, 3 TS muscles) was approximately equal to that observed in nondystrophic TS fibers (−50.1±1.4 mV). In particular, the mdx mouse treated with PDTC between 5 and 13.5 months exhibited an average resting potential at 13.5 months that slightly exceeded nondystrophic levels (−53.3±2.0 mV, N=22 fibers), even though the average resting potential of saline-injected fibers at an average age of 14.6 months was −35.9±1.8 mV (FIG. 17A1). These results show a pronounced beneficial effect of PDTC treatment on the overall health of mdx TS fibers that is consistent with the positive effects of daily PDTC treatment on the survival of striated fibers in the chronically stretched mdx TS.

### Example 5

### Chronic Treatment With Inhibitors of the NFκB Pathway May Significantly Reduce an Index of Whole Body Fatigue in the mdx Mouse

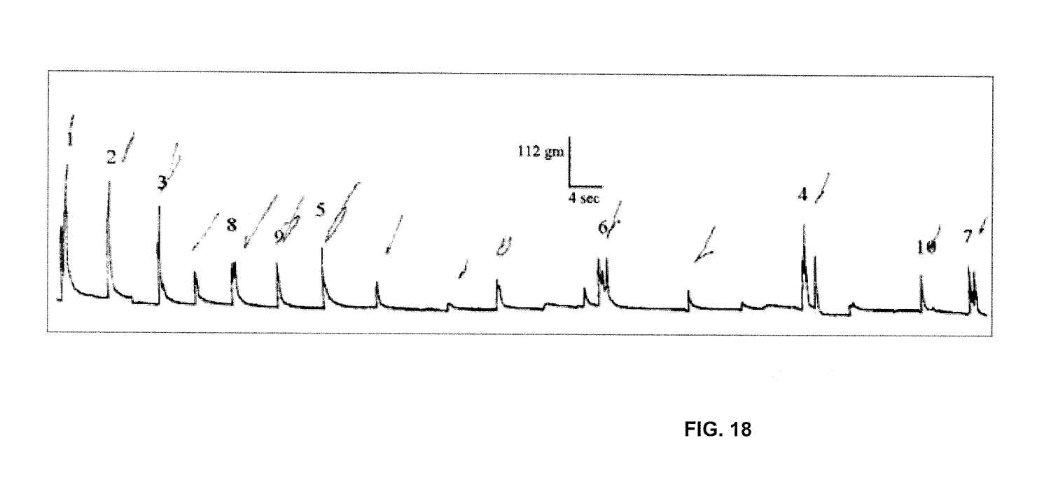

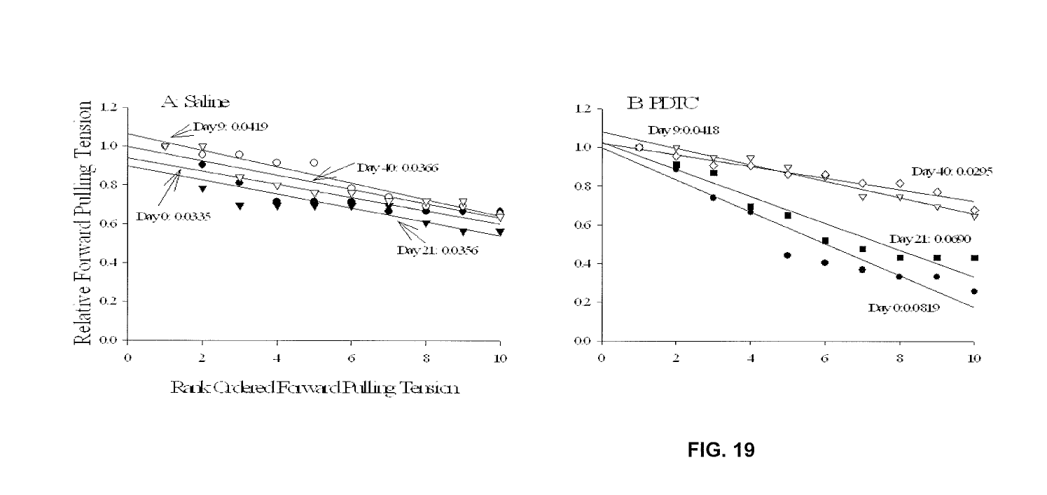

FIG. 18 presents individual “forward pulling tensions” (FPTs; upward deflections) recorded from a PDTC-treated mouse (Series 2) using the whole body tension (WBT) technique (Carlson and Makiejus, 1990). The rank order of FPTs from highest to lowest is indicated by the numbers in the figure. The average of the top 5 and top 10 FPTs divided by the mouse body weight are referred to as the WBT5 and WBT10, respectively, and are significantly reduced in mdx mice in comparison to nondystrophic controls (Carlson and Makiejus, 1990). In the Series 2 experiments, WBT measurements were obtained from each of the saline-injected and PDTC-injected mice on days 0, 9, 21 and 40 of the experiment.

As a first step in examining the potential effects of PDTC treatment on WBT tension development, the slope of the decline in FPTs was evaluated for each recording session for each mouse examined in the Series 2 experiments (FIG. 19A, 19B). This measure essentially provides a “fatigue index” (FI) since the slope of the decline in FPTs is a direct measure of the average magnitude of the decline in FPT per individual pull for rank-ordered pulls number 2 through 10. If 10 individual pulls obtained during the recording session have identical magnitudes representing the highest FPT, then the FI equals 0. In contrast, a 50% reduction in FPT per pull would produce an FI equal to −0.5.

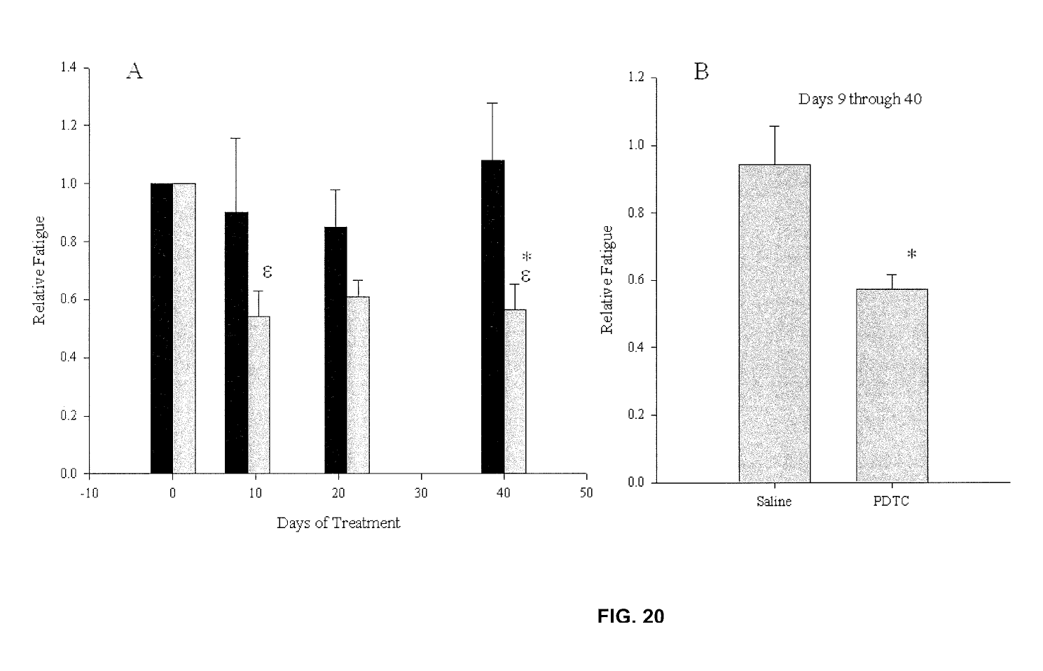

To examine the potential effect of PDTC treatment on FI in age matched adult mdx mice, a pre-treatment FU was obtained during a typical recording session from each of 10 mdx mice used in the Series 2 investigations (age 9.5 to 16 months at beginning of study). Five of the mice were subsequently treated with daily injections of vehicle (HEPES buffered Ringer's solution) and 5 of the mice were treated with daily injections of 50 mg/kg PDTC. Recordings for each of the mice were subsequently obtained on days 9, 21, and 40 of the treatment period (e.g., FIG. 19). To determine the potential effects of treatment on FI, the FIs obtained on days 9, 21, and 40 (e.g., FIG. 19) were each normalized to the pre-treatment FI for each of the 10 mice used in the study. FIG. 20 shows the average normalized FI values for the 5 vehicle-injected and 5 age-matched PDTC-treated mice used in this initial study (FIG. 20A; saline injections-black bars; PDTC injections-gray bars). A decrease in the FI indicating a reduction in the average loss of tension per pull during the course of the study would produce normalized FIs less than one, no change in FI would be associated with normalized FIs equal to 1.0, and increases in FI would produce normalized FIs greater than 1.0.

Daily injections of vehicle did not significantly alter the FI (FIG. 20). In contrast, the PDTC-treated mice showed a significant effect of treatment on the normalized FI (ε, p<0.05; Kruskal-Wallis ranks ANOVA; N=5 mice repetitively sampled) with significant differences observed between Day 0 and Day 9 and between Day 0 and Day 40 (E, Tukey, p<0.05). Direct comparisons between vehicle-injected and PDTC-injected mice failed to reach significance on Days 9 and 21, but were significantly different (*, p<0.05, t test) at Day 40 of treatment (FIG. 20A). When the post-treatment results (Days 9, 21, and 40) were combined for the two groups (FIG. 20B), there was a significant (*, p<0.05; t test) effect of PDTC treatment in reducing the FI. These results provided the first evidence that chronic treatment with inhibitors of the NFκB pathway produced a mild functional benefit by significantly reducing an index of whole body fatigue in the mdx mouse. It indicates that, during a typical 2 to 3 minute WBT recording session, mdx mice treated with inhibitors of the NFκB pathway exhibit less decline in total body strength and thereby produce more consistent FPTs than do age-matched, vehicle-injected mdx mice.

### Example 6

### Chronic Treatment With Inhibitors of the NFκB Pathway Produce Increases in Whole Body Tension in Mature mdx Mice

Carlson and Makiejus (1990) showed that mdx mice as young as 4 to 10 weeks of age exhibit skeletal muscle weakness that can be quantified using a simple noninvasive procedure for assessing whole body strength. In these initial experiments, the top 5 or top 10 FPTs observed during a WBT recording session (FIG. 18) were averaged and divided by the total body weight to obtain noninvasive measures of whole body tension, WBT5 and WBT10, respectively. Mdx mice exhibited significant reductions in WBT5 at all age intervals investigated between 4 weeks and 2 years of age. Although the WBT10 values were not explicitly reported in that initial study, the WBT10/WBT5 ratio was determined as an index of fatigue (“functional reserve”, FR) and shown to be significantly reduced in mdx mice at all age intervals examined (4-10 weeks, 10-20 weeks, >20 weeks).

In the initial series of experiments examining the effects of chronic PDTC administration on WBT in mature mdx mice, 3 separate series of investigations (Series 2, 3, and 4) were conducted using age-matched vehicle-injected and PDTC-injected mice that were older than 5 months of age at the beginning of drug treatment. Series 2 consisted of 10 vehicle-injected and PDTC-injected mice treated initially at 9.5 to 16 months of age. Series 3 mice were treated initially at 19-20 months of age, and series 4 were treated initially at 5 months of age. In each individual series, vehicle and PDTC treated mice were age-matched at the beginning of the study.

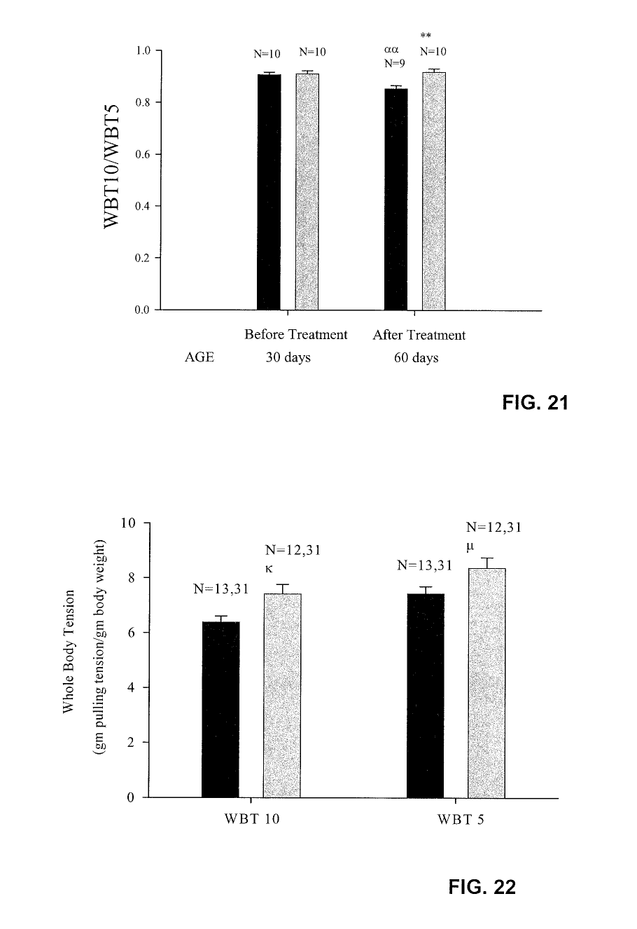

FIG. 21 represents the results from all WBT measurements obtained from all vehicle-injected and PDTC-injected mice in Series 2 through 4. In each case, measurements of WBT5 and WBT10 were obtained prior to the treatment period and on several occasions after at least 20 days of treatment (1 injection per day, et Carlson et al., 2005). FIG. 21 shows the post-treatment results obtained from all the mice in Series 2 through 4 and indicates that PDTC-treated mice exhibited significantly (p<0.05) elevated WBT10 and WBT5 values in comparison to age-matched, vehicle-injected mdx mice. A comparison of post-treatment to pre-treatment values of WBT10 and WBT5 for vehicle-injected mice indicated no influence of vehicle on these variables, while a similar comparison for PDTC-treated mice showed that chronic treatment with the drug significantly (p<0.05; t test) increased both the WBT10 and the FR above pre-treatment levels. These results indicate that chronic treatment with inhibitors of the NFκB pathway produce a mild functional benefit in increasing total body strength in a manner that is consistent with the reduction in FI and enhanced consistency of FPT seen initially in the Series 2 investigations.

### Example 7

### Chronic Treatment With Inhibitors of the NFκB Pathway Prevents Developmental Decreases in Functional Reserve in Young mdx Mice

To further examine the potential functional benefit of treating mdx mice with an agent that stabilizes cytosolic IκBα and inhibits the NFκB pathway, mdx mice at approximately 30 days of age were treated for 30 consecutive days with either vehicle or 50 mg/kg PDTC, and WBT measurements were obtained in each mouse before and after the treatment period. At 30 days of age, mdx mice exhibited a significant reduction in both WBT10 and WBT5 in comparison to nondystrophic mice but had FR (WBT10/WBT5) values that approached those seen in nondystrophic mice. After 30 days of treatment (60 days of age), the vehicle-injected mice showed a significant (αα, p<0.01; t test) age-dependent reduction in FR in comparison to the FR observed in 30 day old mdx mice (FIG. 22). In contrast, mdx mice treated daily with an NFκB inhibitor showed a stable FR of approximately 0.9 that was significantly (**, p<0.01; t test) increased relative to that seen in the vehicle-injected mdx mice at 60 days of age. Although both the WBT10 and WBT5 values were increased relative to the vehicle-injected mice, these increases did not reach statistical significance at 60 days of age. However, the WBT10 value was increased significantly more than the WBT 5 value in the PDTC treated mice, thus producing the significant increase in FR at this age. These results indicate that the FR in mdx mice declines significantly between 1 and 2 months of age and that daily treatment with an inhibitor of the NFκB pathway prevents this decline in muscle function that is a characteristic of Duchenne and related muscular dystrophies.

In summary, chronic treatment of a sample of age-matched mature mdx mice (age 9.5 to 16 months at beginning of treatment period) indicated a significant (p<0.05) improvement in the FI, a measure of the decline in FPT over the top 10 FPTs in the noninvasive procedure for determining whole body tension (Carlson and Makiejus, 1990). This indicates that PDTC-treated mature mdx mice exhibited significant functional improvement characterized by the ability to produce more consistent FPTs over a typical WBT recording session (FIG. 20). Secondly, a larger sample of mature mdx mice exhibited a significant improvement in both WBT5 and WBT10 in comparison to age-matched, vehicle-injected mice (FIG. 21). This indicates that PDTC treatment induced mild but significant increases in total body strength even when administered to mice that were in the advanced stages of the disease. The third observation in these initial studies was that PDTC treatment prevented a decline in the FR when administered to a population of young adult mdx mice (FIG. 22). This indicates that inhibition of the NFκB pathway in young mdx mice prevents a decline in function that is characteristic of Duchenne and related muscular dystrophies. Overall, these results indicate that in vivo inhibition of the NFκB pathway produces a mild functional benefit in increasing total body strength primarily by enhancing the consistency of FPTs in the non-invasive determination of whole body strength (Carlson and Makiejus, 1990).

### Example 8

### Chronic In Vivo Treatment With Inhibitors of the NFκB Pathway Improves Tension Development in Adult Adult MDX Muscle Preparations

To further determine whether treatment with NFκB inhibitors influences skeletal muscle function in dystrophic muscle, mdx mice were treated daily with either PDTC (50mg/kg) or vehicle as previously described (Carlson et al., 2005). In one set of experiments, mature mdx mice (average age 15 months at sacrifice) were treated for at least 2 months (average treatment period=72 days). Following the treatment period, the mice were anaesthetized with sodium pentobarbitol (0.1 mg/kg, ip) and the gastrocnemius preparation (lateral and medial heads, associated plantaris muscle intact) was isolated as initially described in Carlson and Makiejus (1990).

Briefly, the mouse to be examined is placed ventral side down and the gastrocnemius muscle exposed after cutting or reflecting the overlying sartorius muscle. The calcaneous and plantaris tendons are first tied with surgical thread that is tied to a metal hook that fits directly into an isometric tension transducer (Grass Instruments, Model FT03C). After cutting the tendons distal to the ligature, the gastrocnemius, plantaris and soleus muscles are reflected dorsally and the soleus muscle removed from the preparation. The preparation is kept moist with HEPES buffered Ringer solution (in mM: 147.5 NaCl, 5 KCl, 2 CaCl₂, 11 glucose, 5 HEPES, pH 7.35) throughout the surgery and the mouse hindlimb is firmly attached to the surface of a Sylgaard tray using pins (which do penetrate any tissue) or specially constructed hooks. The metal hook and thread are then attached to the isometric tension transducer which is itself attached to a micromanipulator (Narishige) that is used to alter muscle resting length. The muscle is stimulated directly (Grass S9 stimulator) with suprathreshold pulses using 1 inch fine (approximate gauge 26) bipolar silver chloride electrodes that are spaced approximately 5 mm apart on the reflected surface of the muscle preparation. The muscle preparation is mildly stretched while applying 5 to 10 individual pulses (4 msec) of increasing intensity to determine the intensity that produces an asymptotic maximum in twitch tension. The preparation is then stimulated with approximately 10 to 20 individual pulses at this suprathreshold intensity while the muscle length is systematically altered to determine the optimal length for maximal tension development (Iₒ). During this time period and throughout the recording session, the muscle is periodically moistened (about every 2 minutes) with HEPES Ringer and the depth and frequency of respiration is noted. In the rare cases in which the mouse was initially over-anesthetized (<=5% of experiments in practice), a rapid fatigue of tension development was observed signaling a decline in cardiac output during the recording session. Such preparations which did not exhibit stable twitch amplitudes under non-fatiguing stimulation (e.g., 1 pulse 30 seconds) were discarded from further analyses. In rare circumstances, preparations initially exhibited stable twitch amplitudes and subsequently exhibited declining tetanic tensions that suggested a decline in cardiac output. Under these circumstances, the tetanic tension data was discarded from further analyses.

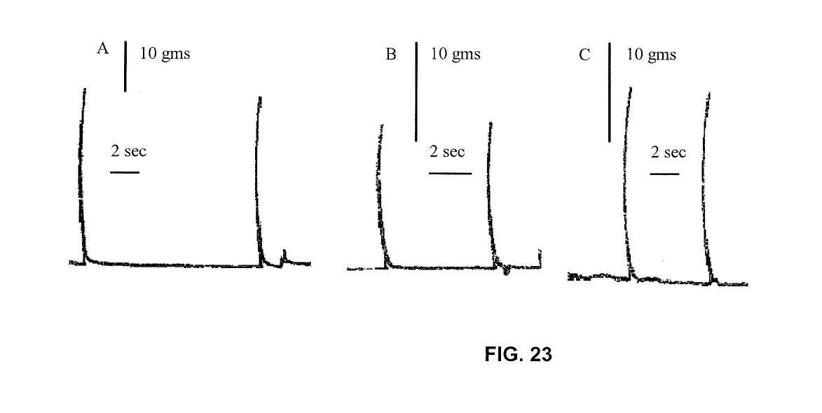

Optimal length was determined by carefully measuring increasing tensions as the muscle length was increased along the ascending limb of the length-tension curve and then noting a 5 or 10% decline in tension as the muscle was lengthened beyond lₒ. At this point, the muscle length was shortened back to lₒ and stimulated approximately 5 times (with appropriate rest periods between stimulations) to determine the twitch tension at lₒ (FIG. 23). After determining twitch tension at lₒ, the preparation was stimulated briefly (1-2 minutes) at frequencies of 0.2, 0.5, and 1.0 Hz to assess the stability of twitch tension. Stimulation was then applied at 10 Hz for a period of 20 to 60 sec to assess the decline in twitch tension at this frequency. The subsequent rate of recovery of twitch amplitude over a period of approximately 1 to 3 minutes following 10 Hz stimulation was also determined before assessing twitch:tetanus ratios (about 1-2 sec tetanic stimulation) at both 30 and 50 Hz. Finally, the decay of twitch amplitude at 10 Hz and the subsequent recovery of twitch tension was assessed two more times before obtaining plasma samples and euthanizing the animal.

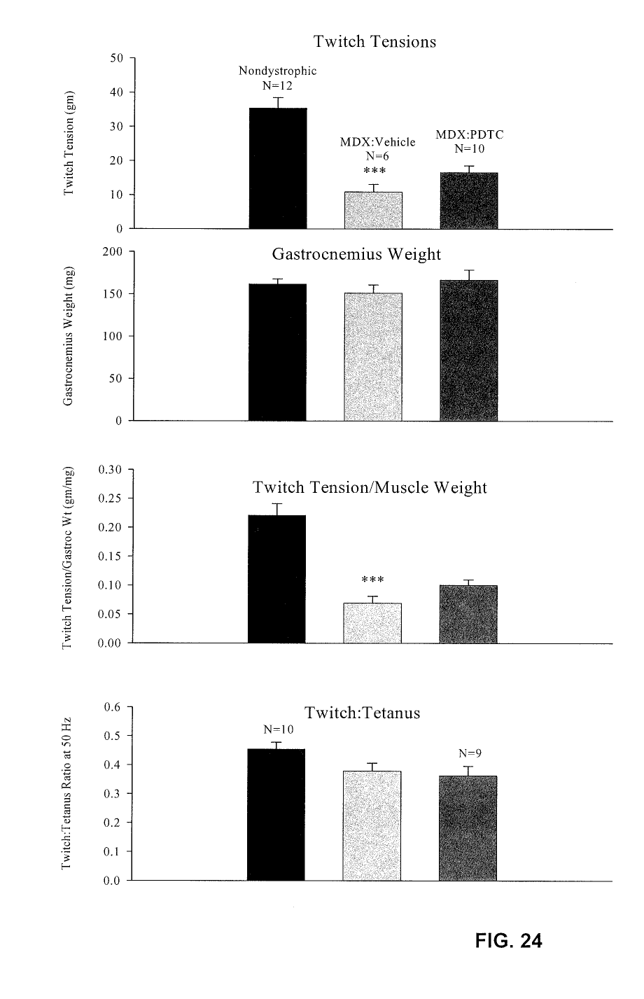

As initially reported in Carlson and Makiejus (1990), in comparison to age-matched nondystrophic mice, mature vehicle-injected mdx mice (average age about 15 months, Series 2-4; cf Carlson et al., 2005) exhibited highly significant (p<0.001) decreases in twitch tension (gm) and specific twitch tension (gm tension/mg muscle wet weight) to approximately 30% of nondystrophic values with no significant changes in gastrocnemius weight or in twitch/tension ratio (FIG. 24). Mature, PDTC-treated mice exhibited a 52% improvement (relative to vehicle-injected mice) in the average twitch tension and a 45% improvement in twitch tension/gm that just failed to reach statistical significance (p>=0.05).

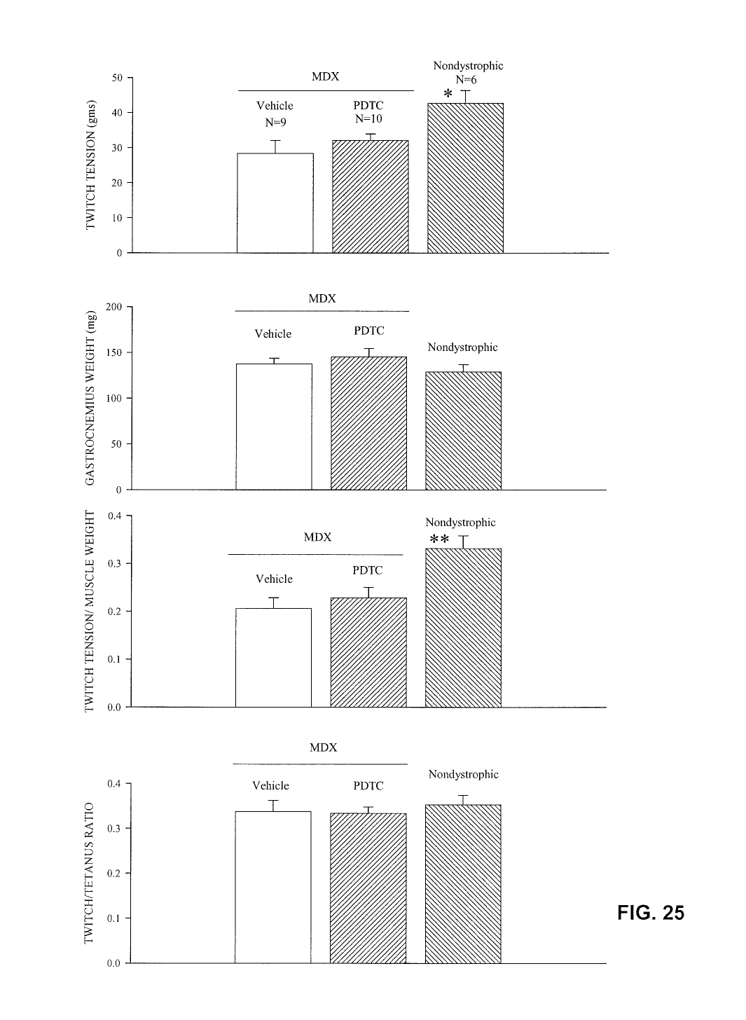

Similar results were obtained from young adult mice (2 month old). In this case, the mdx twitch tensions and twitch tension/gm muscle weight were significantly reduced to 66% (p<0.05) and 62% (p<0.01), respectively, of the corresponding nondystrophic values (FIG. 25). In comparison, the mature (15 month) mdx preparations exhibited twitch and twitch/muscle weight values of approximately 30% of nondystrophic levels (FIG. 24). These results indicate that the mdx gastrocnemius muscle progressively weakens as a consequence of the dystrophic process, similar to what is observed in Duchenne muscular dystrophy. This conclusion indicates that the mdx mouse is a functionally valid model for studying the potential utility of proposed treatments for Duchenne and the related muscular dystrophies (Carlson and Makiejus, 1990). In comparison to mdx vehicle-treated mice, PDTC treatment produced a 13% increase in twitch tension and an 11% increase in twitch tension/mg muscle weight in the young adult preparations (FIG. 25).

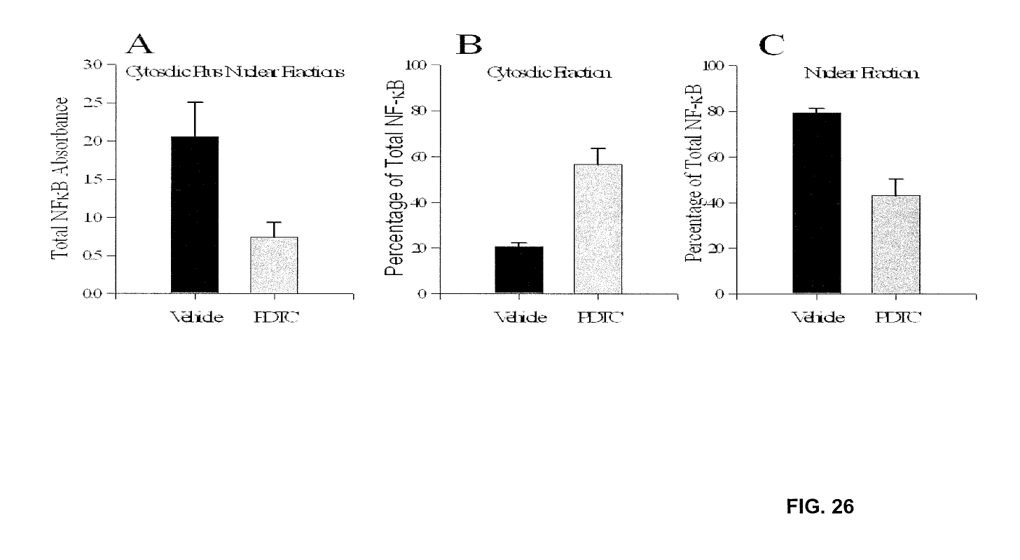

The results of Trans AM assays demonstrate that chronic treatment of adult mdx mice with PDTC reduces total cellular NFκB and the proportion of nuclear NFκB in mdx skeletal muscle (FIG. 26). The Trans AM assay and corresponding electrophoretic mobility shift assays (EMSA), along with Western Blot analyses of IκB-α (FIG. 1) and total cellular NFκB, are useful for screening compounds that inhibit the NFκB pathway in dystrophic skeletal muscle and identifying new compounds that have corresponding beneficial effects in treating dystrophic subjects.

### Example 9

### Cytosolic Extracts of Adult MDX Skeletal Muscle Exhibit Significant Increases in Cytokine Expression in Comparison to Nondystrophic Preparations

To determine whether cytosolic extracts from dystrophic muscle exhibit elevated levels of cytokines that are regulated by NFκB, the diaphragm muscles were removed from euthanized mature nondystrophic (C57Bl10SnJ) and mdx mice, and cytosolic and nuclear extracts were obtained from this muscle tissue using procedures that were slightly modified from those described in Carlson et al. (Neurobiology of Disease, 20, 719-730, 2005). In these experiments, the nondystrophic mice were between 8 and 31 months of age at euthanasia (average age—18.4 months) and the mdx mice had an average age of 14.6 months. Each diaphragm was divided into costal and crural regions which were processed and analyzed separately. After determining protein concentrations for each of the cytosolic and nuclear extracts (Lowry assay), samples of each extract were used to obtain standard ELISA determinations of mouse TNFα, IL-6, and IL-1β using standard procedures (Assay Designs, Inc.). These particular NFκB products were chosen because of their role in the inflammatory reaction and previously published results indicating the presence of these cytokines in nondystrophic skeletal muscle homogenates (Molina et al., Neuroimmunomodulation, 4, 28-36, 1997; Jonsdottir et al., J. of Physiol. (Lond.), 528.1, 157-163, 2000; Lang et al., Shock, 19(6), 538-546, 2003).

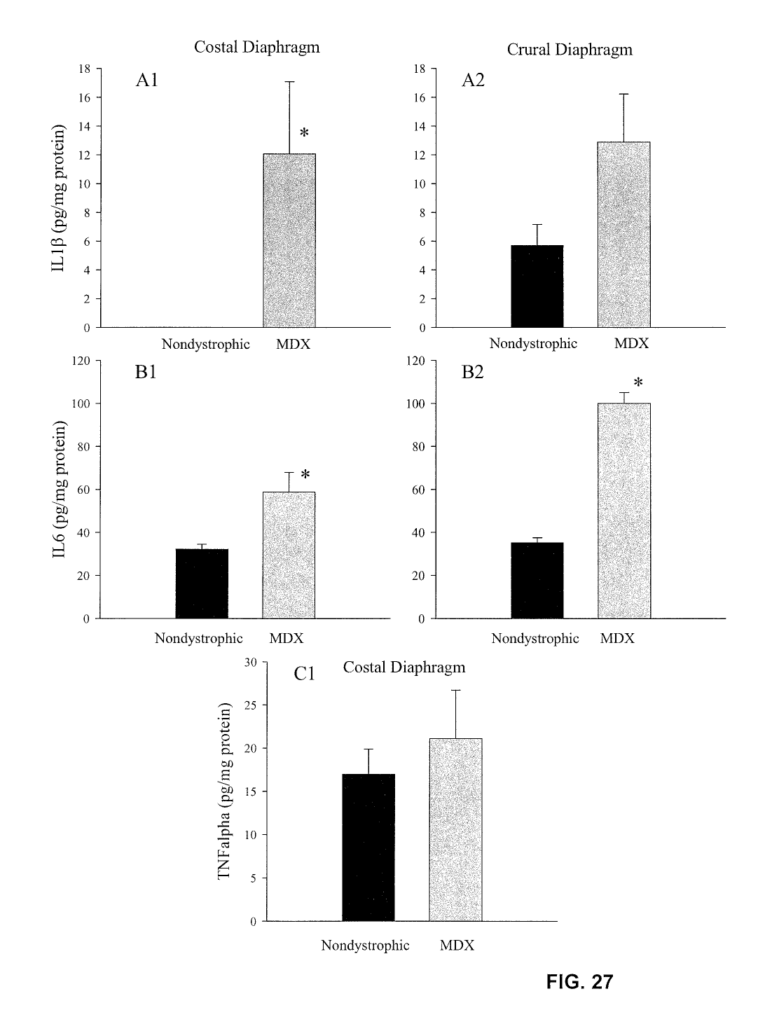

FIG. 27 (ioncluding parts A1, A2, B1, B2, C1) shows that the cytokines TNFα, IL-6, and IL-1β were each present in conventional cytosolic extracts of skeletal muscle and that freshly excised costal and/or crural diaphragms from mdx mice exhibited statistically significant (p<0.05) increases in IL-1β and IL6 in comparison to corresponding adult nondystrophic preparations. The levels of TNFα were also elevated in the costal mdx diaphragm but this increase did not reach statistical significance (p>0.05).

### Example 10

### Sulfasalazine Treatment In Vivo May Reduce Skeletal Muscle Nuclear Activation of NFκB in Dystrophic (mdx) Skeletal Muscle

MDX mice aged 3 to 3.5 months were injected with either sulfasalazine (SS; 100 mg/kg, ip) or vehicle (HEPES Ringer; in mM: 147.5 NaCl, 5 KCl, 2 CaCl2, 11 glucose, 5 HEPES, pH 7.35) and euthanized at 3 hours post-injection. The costal diaphragms were removed and flash frozen before beginning the extract procedure. Nuclear and cytosolic extracts were obtained using a procedure slightly modified from that described in Carlson et al. (2005) and Kumar and Boriek (2003). Briefly, the muscles were weighed after removing tendinous components, and frozen and homogenized by mortar and pestle in low salt lysis (LSL) buffer on ice (1 mg muscle wet weight/18 μl solution; in mM: 10 HEPES, 10 KCl, 1.5 MgCl₂, 0.1 EDTA, 0.1 EGTA, 1 dithiothreitol (DTT), 0.5 phenylmethylsulfonylfluoride (PMSF); 0.5 mg/ml benzamidine, 4.0 μl/ml protease inhibitor cocktail Sigma #8340 (PIC) to produce the following final concentrations—2.1 μg/ml leupeptin, 3.85 μg/ml aprotinin, 0.416 mM AEBSF (Sigma A8456), 16 μM bestatin, 6 μM pepstatin A, 5.6 μM E64; pH 7.9). To lyse the cells, the ground tissue was subjected to 2 freeze-thaw cycles (5 minute freeze on dry ice followed by thawing at room temperature), and subsequently vortexed and centrifuged (13,000 rpm, 15 sec). The supernatant cytosolic extract was immediately frozen (−80° C.) for subsequent analyses as needed, while the nuclear pellet was washed one time with 500 μl of low salt lysis buffer before being resuspended on ice in a high salt nuclear extraction buffer (in mM: 20 HEPES, 420 NaCl, 1 EDTA, 1 EGTA, 1 DTT, 0.5 PMSF; 25% glycerol, 0.5 mg/ml benzamidine, 4.0 μl/ml PIC; pH 7.9) at a ratio of 4 μl of solution per mg muscle wet weight. The suspension was incubated on ice for 30 minutes and vortexed for 10 sec every 5 minutes before being centrifuged (13,000 rpm, 6 minutes) at 4° C. The supernatant nuclear extract was then removed and frozen (−80° C.) for the subsequent EMSA which was performed within 5 days following the in vivo treatment. Corresponding experiments to determine potential contamination of nuclear extracts with cytoplasmic proteins were performed by determining cytokine levels in both nuclear and cytosolic extracts of dystrophic muscle (ELISA assay). The results of these experiments indicated essentially zero contamination of the nuclear extracts by cytoplasmic proteins. Protein determinations were determined by the method of Lowry using bovine serum albumin standards (20 μg nuclear protein was added to each lane).

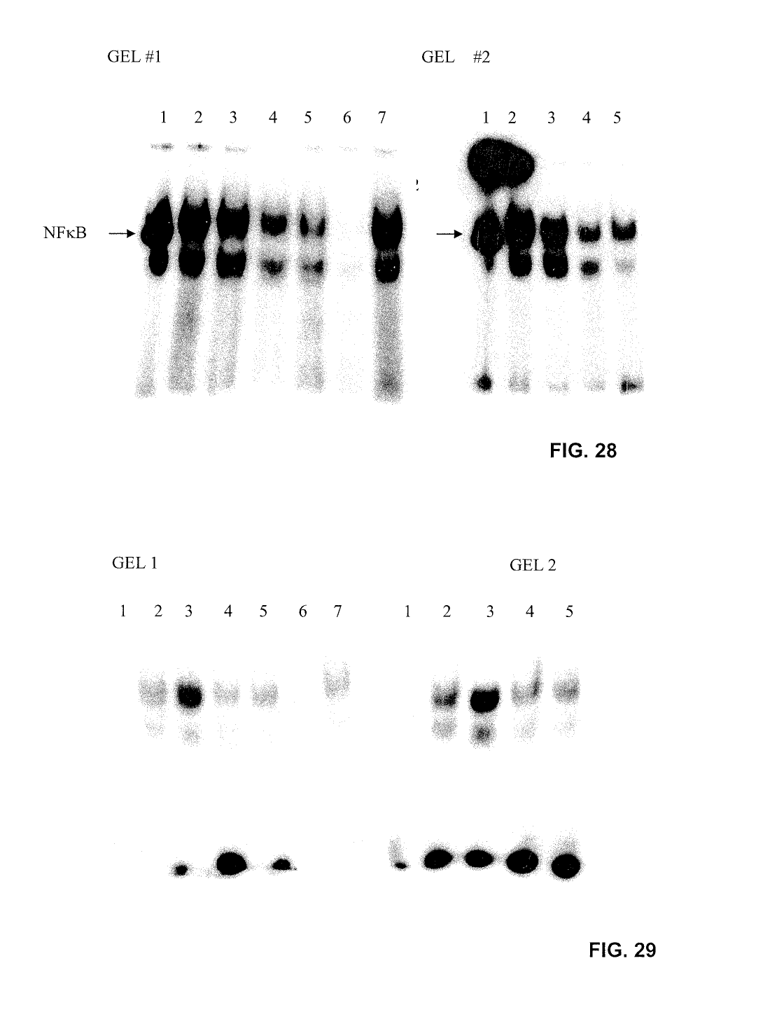

The results shown in FIG. 28 indicate a reduction of nuclear NFκB binding to the appropriate consensus sequence in the SS treated mice (compare lanes 4 and 5 to lanes 2 and 3). Based upon the therapeutic effects of chronic PDTC treatment (Carlson et al., 2005), which reduced total cellular NFκB and the proportion of nuclear NFκB in mdx skeletal muscle (FIG. 26) by stabilizing cytosolic IκB-α (FIG. 1), these results showing an acute effect of SS on the nuclear activation of NFκB in dystrophic muscle directly indicate a therapeutic benefit in treating muscular dystrophy.

### Example 11

### Parthenolide Treatment In Vivo Reduces Skeletal Muscle Nuclear Activation of NFκB in Dystrophic (mdx) Skeletal Muscle

MDX mice, aged 3 to 3.5 months, were injected with either parthenolide (5 mg/kg in vehicle) or vehicle alone (HEPES Ringer; in mM: 147.5 NaCl, 5 KCl, 2 CaCl2, 11 glucose, 5 HEPES, pH 7.35; 0.1% DMSO) and euthanized at 3 hours post-injection. The costal diaphragms were removed and flash frozen before beginning the extract procedure. Nuclear and cytosolic extracts were obtained as described in Example 10. Nuclear extracts were used for EMSA assay as described in Example 10. Protein determinations were determined by the method of Lowry using bovine serum albumin standards (20 μg nuclear protein was added to each lane).

Parthenolide is a member of the class of sesquiterpene lactones that inhibits NFκB activation by a variety of mechanisms including stabilization of cytosolic IκB-α (Hehner et al., J. Biol. Chem., 273(3), 1288-1297, 1998; Wong and Menendez, Biochem. Biophysica Res. Comm., 262, 375-380, 1999), inhibition of IKK (Kwok et al., Chem and Biol., 8, 759-766, 2001), and inhibition of the binding of NFκB to the κB consensus sequence (Sheehan et al., Molec. Pharmacol., 61, 953-963, 2002). The results shown in FIG. 29 were analyzed densitometrically to indicate that the single injection of PTN reduced the nuclear activation of NKκB by 50% in comparison to the vehicle injected dystrophic mice (compare lanes 4 and 5 to lanes 2 and 3). These results provide the first demonstration that parthenolide and other sesquiterpene lactones inhibit the nuclear activation of NFκB in dystrophic skeletal muscle following a single in vivo injection. Based upon the therapeutic effects of chronic PDTC treatment (Carlson et al., 2005), which reduced total cellular NFκB and the proportion of nuclear NFκB in mdx skeletal muscle (FIG. 26) by stabilizing cytosolic IκB-α (FIG. 1), these results showing an acute effect of parthenolide and other sesquiterpene lactones on the nuclear activation of NFκB in dystrophic muscle provide the first direct indication of the therapeutic utility of this class of substances in treating muscular dystrophy.

### Example 12

### Sulfasalazine Treatment In Vivo May Reduce Cytokine Expression in Some Dystrophic (mdx) Skeletal Muscles

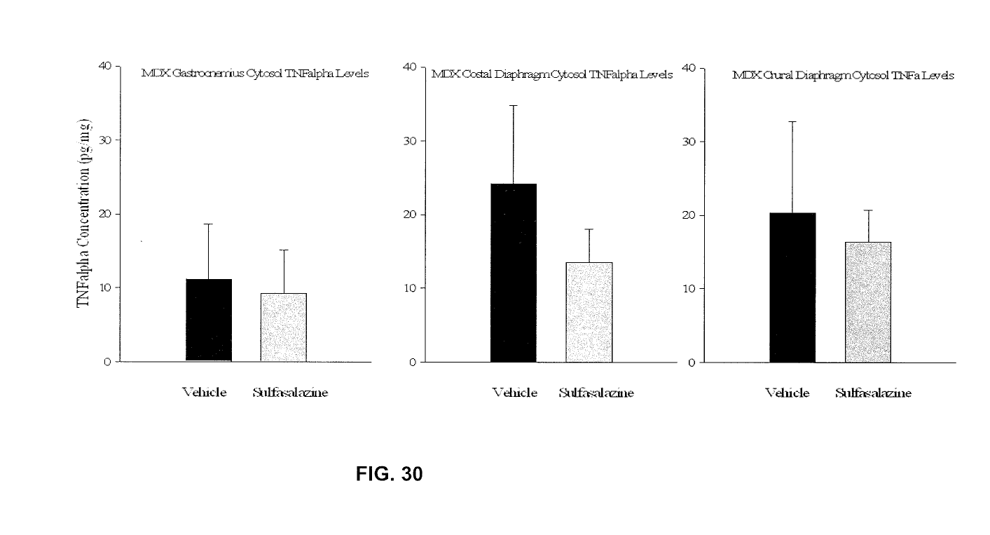

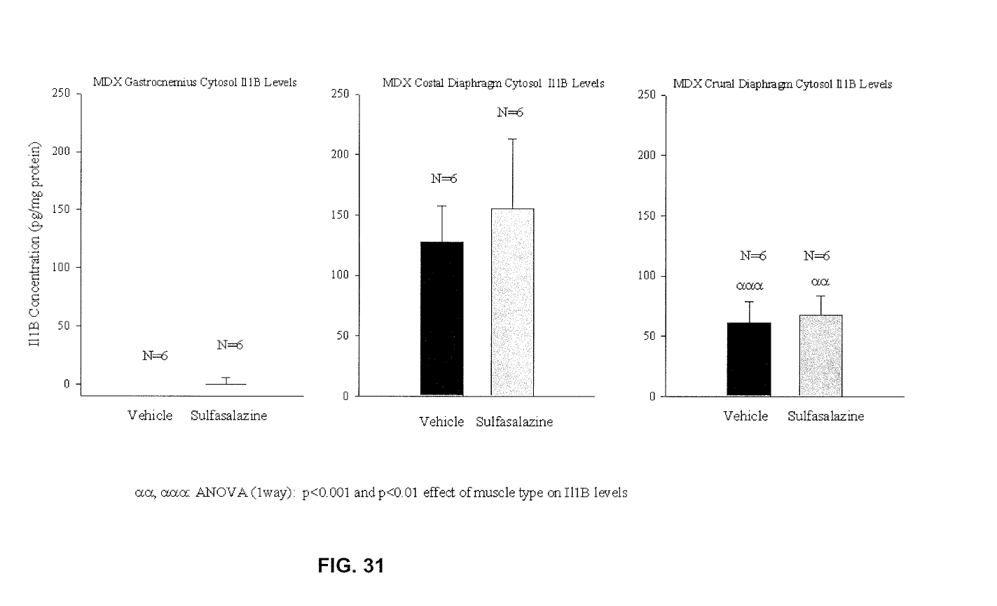

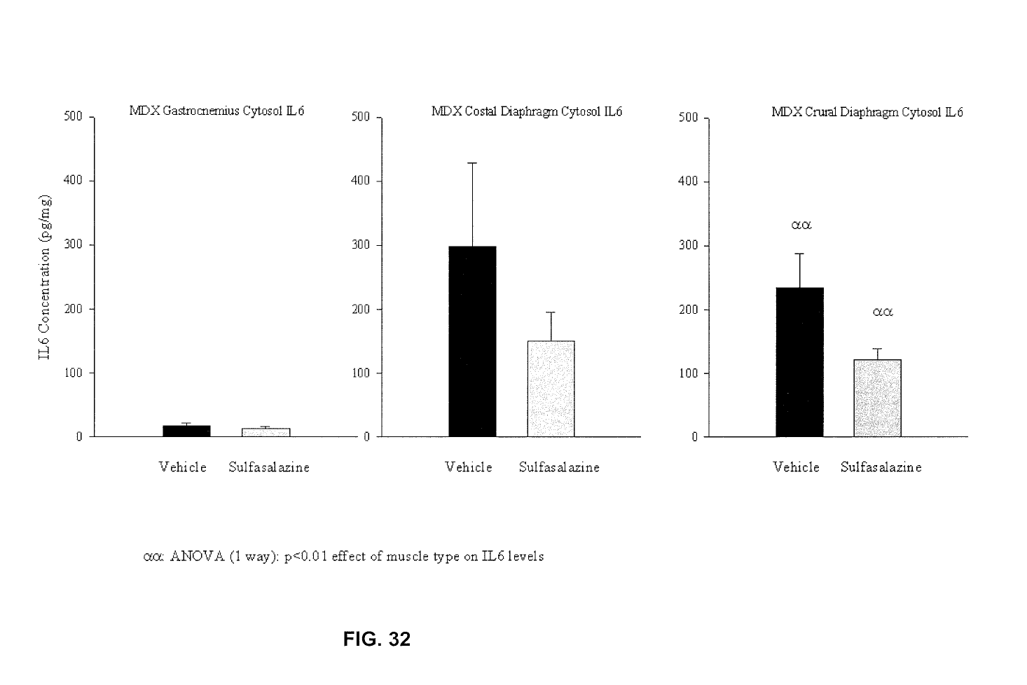

MDX mice aged 3 to 3.5 months were injected with either SS (100 mg/kg) or vehicle (HEPES Ringer; in mM: 147.5 NaCl, 5 KCl, 2 CaCl2, 11 glucose, 5 HEPES, pH 7.35) and euthanized at 3 hours post-injection. The costal and crural diaphragms and the gastrocnemius muscle were then immediately removed and flash frozen before beginning the procedure to obtain nuclear and cytosolic extracts as described in Example 10. ELISA determinations of TNFα, Il1-β, and IL6 were determined as described in Example 9. FIG. 30 shows that mdx gastrocnemius, costal diaphragm, and crural diaphragms exhibited roughly equivalent expression of TNFα and that a single injection of sulfasalazine reduced the expression of TNFα in the cytosolic extracts from mdx costal diaphragm. FIG. 31 shows that the expression of IL1-β was significantly increased in both the costal and crural diaphragms in comparison to the gastrocnemius muscle which did not exhibit IL1-β expression. This observation suggests that the expression of IL1-β may contribute to the more severe phenotype characteristic of mdx diaphragm muscle. A single injection of sulfasalazine did not reduce the expression of IL1-β. However, a single injection of sulfasalazine did reduce the expression of IL6 in both the costal and crural diaphragms (FIG. 32) suggesting that the significantly (p<0.01) elevated expression of this cytokine in mdx diaphragm in comparison to gastrocnemius may contribute to the dystrophic phenotype. These results are consistent with the results showing that a single sulfasalazine injection reduces the nuclear activation of NFκB (FIG. 28), and further suggest that the potential utility of various compounds in treating muscular dystrophy may be assessed by determining the acute and chronic effects of the compound on cytokine expression in a variety of dystrophic muscle samples.

### Example 13

### Chronic In Vivo Treatment with Sulfasalazine Significantly Improves the Resting Membrane Potential in Dystrophic (mdx) Triangularis Sterni (TS) Muscle Fibers

Sulfasalazine (“SS”) (Sigma Number S-0883) was dissolved in standard HEPES Ringer solution (pH 7.35) at a concentration of 10 mg/ml by adding a few drops of 0.5 M NaOH until the solution turned a clear pink or red indicating that the sulfasalazine powder had dissolved (pH approximately 10.0). An equal amount of 0.5 M NaOH was then added to an equal volume of vehicle. These solutions were immediately used to inject one sulfasalazine-treated mouse (ip) with 100 mg/kg of the sulfasalazine solution (0.3 ml for a 30 gm mouse) and another littermate with an equivalent volume of vehicle solution. Aside from a brief (5 to 10 sec) period of restlessness immediately following the injection, the mice displayed no obvious side effects from either the vehicle or SS injection. This procedure was repeated with fresh solutions on a daily basis for 68 (SS treated) and 70 (vehicle treated) consecutive days when each mouse received a final injection prior to being euthanized. The mice maintained their body weight and displayed no obvious side-effects to this chronic treatment schedule. After euthanizing the mice, the TS muscles were removed and intracellular recordings of resting potential were obtained using the techniques described in Carlson et al. (2005).

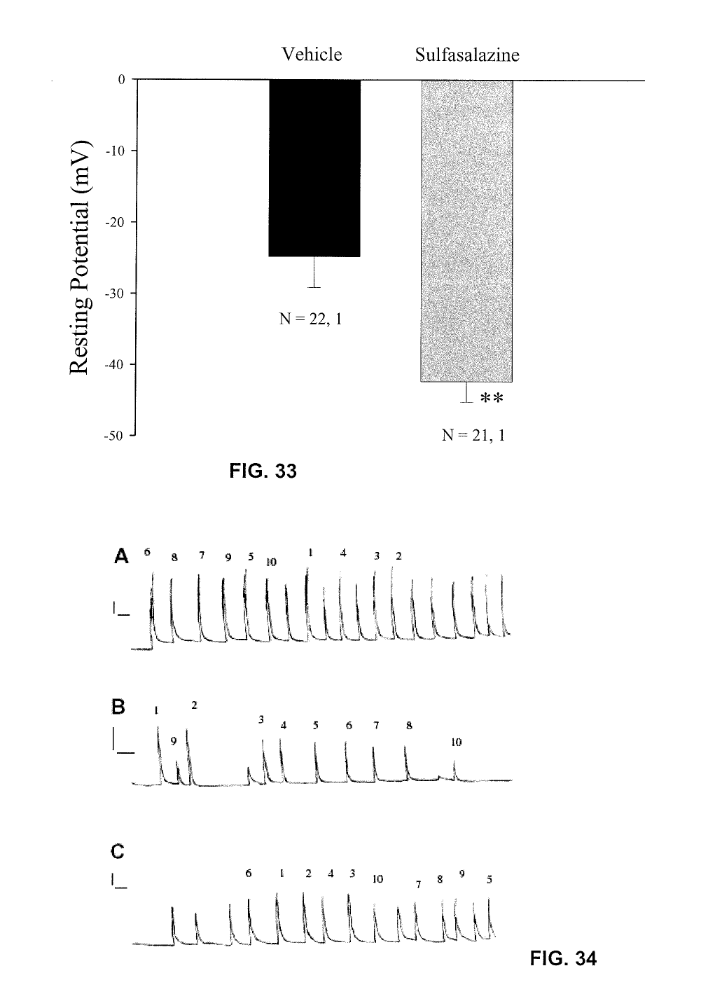

The results indicate that chronic treatment with SS significantly improved the resting potential of TS muscle fibers (FIG. 33) in a manner consistent with previous results obtained using chronic PDTC treatment (FIGS. 16, 17). The results indicate that chronic treatment with inhibitors of the NFκB pathway such as sulfasalazine have beneficial effects in treating muscular dystrophy (Carlson et al., 2005). Sulfasalazine had previously been shown to inhibit the NFκB pathway in a variety of human tissues (See Wahl et al., 1998; Gan et al., 2005; Lappas et al., 2005; and Robe et al. 2004). The results of electrophoretic shift assays (EMSA) following a single injection of SS provided the first demonstration that this drug effectively blocks the NFκB pathway in dystrophic muscle (FIG. 28). The available evidence indicates that sulfasalazine stabilizes IκB-α (Wahl et al., 1998) and inhibits IKK (Weber et al., 2000) in other tissues.

SS is used at similar doses in treatment of rheumatoid arthritis and ulcerative colitis. The results described here indicate that chronic treatment with SS has beneficial effects in improving the electrical characteristics of dystrophic muscle fibers by improving the resting membrane potential as initially demonstrated using the IκB-α stabilizing agent, PDTC (Carlson et al., 2005). These results also provide the first evidence that this drug may reduce nuclear NFκB activation in dystrophic muscle (FIG. 28) and that chronic treatment may substantially improve the electrical characteristics of dystrophic muscle fibers (FIG. 33). These results provide the first indication supporting the use of sulfasalazine in human clinical trials to treat Duchenne and related muscular dystrophies.

### Example 14

### Agents which Inhibit p65 Transactivation are More Effective at Reducing Nuclear NF-kappaB Activation and Enhancing Limb Muscle Function than Agents which Stabilize Cytosolic IkappaB-Alpha

### PDTC and UDCA Treatment

Mdx (C57 Bl10-mdx) and nondystrophic (C57Bl10-SnJ) mice were obtained from Jackson laboratories and bred under conditions approved by the Institutional Animal Care and Use Committee (IACUC). All studies were performed in accordance with the guidelines provided by the IACUC. Male and female PDTC treated mdx mice received daily intraperitoneal (ip) injections of 50 mg/kg pyrollidine dithiocarbamate (PDTC; Sigma P8765) dissolved in sterile-filtered (0.2 μm) HEPES-buffered Ringers solution (147.5 mM NaCl, 5 mM KCl, 2 mM CaCl₂, 11 mM glucose, 5 mM Hepes, pH 7.35). Age- and gender-matched groups of vehicle-treated mice received corresponding injections of HEPES Ringers. See Carlson et al., 2005. The PDTC experiments were conducted on two series of mature adult mdx mice (9.5-16 month; 19-20 months) and on a series of young adult (1 month) mice. In each case, the mice in the experimental and vehicle-treated groups were matched according to age and gender with an equal number of males and females in each group. WBT tension measurements were obtained prior to initiating treatment and on multiple occasions following at least 3 weeks (20 days) of treatment.

The results of PDTC treatment on WBT and other measurements of limb muscle function are described in Examples 5 through 7 of this disclosure. To compare the therapeutic utility of PDTC to NF-kappaB inhibitors that function by decreasing the nuclear transactivation of p65, the effects of PDTC on WBT and nuclear p65 activation were compared to corresponding results using the drug ursodeoxycholic acid (UDCA) which has been shown in other systems to directly reduce nuclear p65 transactivation without changing the cytosolic concentration of IkappaB-alpha (Miura et al., 2001).

A group of 30 day-old mdx mice were exposed to daily ip injections (30 day) of 40 mg/kg UDCA (Sigma #5127; 2 mg/ml; N=12 experimental) in an isotonic saline vehicle (1.02% NaCl, pH 8.4,) or a synthetic taurine derivative of UDCA (tauroursodeoxycholic acid, TUDCA, SpA Laboratorio Farmacologico, Cenate Sotto, Italy; 6 mg/ml; N=4 experimental) administered in a saline vehicle (NaCl 6 g/l, KCl 0.3 g/l, CaCl₂ 0.2 g/l, Na acetate 4 g/l, 300 mosM/l, pH 6.1). No differences in the effectiveness of the two closely related compounds was observed and the results were therefore combined and compared to a group of age- and gender-matched mdx mice treated with the appropriate vehicle solution.

### Noninvasive Measure for Assessing Whole Body Skeletal Muscle Function in Mice

WBT measurements were obtained as indicated in Examples 5 through 7 by placing an approximately 0.5 inch square piece of adhesive tape around the tail about 1 inch from the tip. See Carlson and Makiejus (1990). A small (2 cm) alligator clip was then attached to the adhesive tape around the tail and a second piece of adhesive was secured around the alligator clip to prevent slippage. The alligator clip was pre-fastened to a 3 inch long thread of 30 gauge flexible steel wire. The opposite end of the wire was attached to a metal hook that fitted directly into a Grass Force displacement transducer (FT103C) and the mouse was positioned at the mouth of a polyvinyl chloride tube (approximately 1 ft. long) that had been lined interiorly with a conventional aluminum screen (14 Mesh, 0.020 in.). The diameter of the tube was 1.5 inches for mature mice and 0.5 inches for the young adult mice (30-60 days of age). The procedure of attaching the clip to the mouse generally took less than 1 min.

After attaching the thread to the tail of the mouse, the mouse was gently led into the dark interior of the tube. The thread was attached to the transducer and the position of the transducer adjusted until the thread was taut. The tail was gently stroked with serrated forceps to cause the mouse to try to escape and the resultant increase in tension produced by each forward pull (“Forward Pulling Tension”, FPT) was recorded using a Grass Model 7 polygraph. Approximately 15-20 FPTs were recorded during each session. FPTs were expressed in grams and were divided by body weight to obtain WBT. A typical recording session was completed in approximately 5 min. The measurements were obtained during both the morning and afternoon hours from mice maintained on a 12:12 light dark cycle. In most cases, measurements were obtained from all relevant experimental groups (e.g. nondystrophic and mdx) during identical sessions.

Four parameters were routinely obtained from each WBT recording session. The mean force of the five maximal FPTs divided by the body weight is the WBT5, while the corresponding value for the top 10 FPTs is the WBT10. Carlson and Makiejus (1990). In addition, the ratio of the WBT10 to the WBT5 value is a measure of the consistency of FPT generation during a recording session and is termed the “Functional Reserve” (FR; see e.g., Carlson, and Makiejus (1990)). A second measure of FPT consistency is the proportional decline in FPT per pull for the top 10 ordered FPTs (PDP). As defined In Example 5 above, the parameter PDP may also be termed the “fatigue index” or FI. To obtain the PDP, each of the top 10 FPTs was divided by the maximal FPT and a linear regression of these normalized FPTs was obtained. The negative slope of this relationship (FPTₓ/FPT₁ vs ordered pull number, where x is ordered pull number 1 through 10 and FPT₁ is the maximal FPT) is equal to the average proportional decline in FPT per pull for ordered pulls 2 through 10.

### Cytosolic and Nuclear Skeletal Muscle Fractions

Cytosolic and nuclear fractions were obtained from isolated costal diaphragm using techniques that were slightly modified from Kumar and Boriek (2003). The muscles were removed from vehicle and drug-treated euthanized mdx mice at the end of the treatment period, and were flash-frozen and stored at −80° C. for a period of 1 to 4 weeks. The muscles were then thawed and weighed before being homogenized on ice using a mortar and pestle in low salt lysis (LSL) buffer (1 mg muscle wet weight/18 μl solution; in mM: 10 HEPES, 10 KCl, 1.5 MgCl₂, 0.1 EDTA, 0.1 EGTA, 1 dithiothreitol (DTT), 0.5 phenylmethylsulfonylfluoride (PMSF); 0.5 mg/ml benzamidine, 4.0 μl/ml protease inhibitor cocktail Sigma #8340 (PIC) to produce the following final concentrations—2.1 μg/ml leupeptin, 3.85 μg/ml aprotinin, 0.416 mM AEBSF (Sigma A8456), 16 μM bestatin, 6 μM pepstatin A, 5.6 μM E64; pH 7.9). To lyse the cells, the ground tissue was then subjected to 2 freeze-thaw cycles (5 min freeze on dry ice followed by thawing at room temperature), and was subsequently vortexed and centrifuged (13,000 rpm, 15 s). The supernatant cytosolic extract was immediately frozen (−80° C.) for subsequent analyses as needed, while the nuclear pellet was washed in 50 μl LSL (2 times) before being re-suspended on ice in a high salt nuclear extraction buffer (in mM: 20 HEPES, 420 NaCl, 1 EDTA, 1 EGTA, 1 DTT, 0.5 PMSF; 25% glycerol, 0.5 mg/ml benzamidine, 4.0 μl/ml PIC; pH 7.9) at a ratio of 4 μl of solution per mg muscle wet weight. Preparations were incubated on ice for 30 min and vortexed for 10 s every 5 min before being centrifuged (13,000 rpm, 6 min) at 4° C. The supernatant nuclear extract was then removed and frozen (−80° C.) for subsequent analyses. Potential contamination of nuclear extracts with cytoplasmic proteins was less than 5%. Protein determinations for each fraction were obtained using the Bradford assay.

### TransAM NF-κB ELISA Assay

The Trans-Am assay (Active Motif; Carlsbad, Calif.) was used to determine nuclear NF-κB activation in nuclear fractions which were applied to the 96 well ELISA plates at 2-20 μg protein. The p65 primary antibody (catalogue number 40096) was used to detect p65 bound to oligonucleotide in vehicle and drug-treated preparations in accordance with kit instructions. A nuclear extract from Jurkat cells was routinely run as a positive control in association with each set of control and experimental samples.

### Statistical Analysis

Sigmaplot 9.0 and Sigmastat v 3.0 and 3.1 were used. The means and standard errors were evaluated using t-tests or Mann-Whitney Rank Sums tests and one way ANOVA or Kruskall-Wallis one way ANOVA on Ranks (Sigma Stat 3.1) as appropriate. Direct comparisons between age-matched groups of vehicle vs drug-treated mdx mice or untreated nondystrophic vs mdx mice were evaluated using t-tests or Mann-Whitney Rank Sums tests. Age-related changes in WBT variables were evaluated using one way ANOVA or Kruskall-Wallis one way ANOVA on ranks followed by direct comparisons between groups using Tukey's test or Dunns test, as appropriate. Significance was defined at the 0.05 level.

TABLE 1
Evaluation of the WBT5 indicates the overall reproducibility and
consistency of the whole body tension method for assessing the total body
strength of adult nondystrophic mice.

| | Age | | N | WBT 5 |
|---|---|---|---|---|
| Carlson and Makiejus (1990) | 1-2-5 | months | 56.42 | 13.8-14.1 |
| | 2.5-5 | months | 24.17 | |
| | 5-11 | months | 17.7 | |
| Makiejus et al. (1991) | >5 | months | 7.17 | 13.2 |
| Hudecki et al. (1993) | 4-5 | months | 15 | 14.6 |
| | 5-6 | months | 15 | 15.6 |
| Deconinck et al. (1997) | 3-4 | months | 9 | 14.5 |

The results obtained from four different laboratories indicate that nondystrophic mice exert WBT5 values of approximately 13-16 times body weight over a wide range of ages. N values represent the number of determinations and the number of mice used in each study.

### WBT Determinations Yield Several Discrete Assessments of Whole Body Function Throughout the Lifespan of Nondystrophic and mdx Mice

In the initial studies examining muscle weakness in the mdx mouse, WBT measurements indicated a significant (p<0.01) reduction in WBT5 in a sample of 13 mdx mice and 42 nondystrophic mice as early as 4 to 10 weeks of age. In the present report, WBT determinations were obtained on a larger sample of male and female mdx (N=114) and nondystrophic (N=56) mice that encompassed the entire lifespan (30 days to 2 years of age). Since no gender-specific differences were observed, the results from males and females were combined.

While a small percentage of FPTs within any recording session occurred without stimulation (i.e., at the discretion of the mouse), most (>90%) were induced by the experimenter by a gentle stroke of the tail using a pair of serrated forceps. These strokes were applied at the discretion of the experimenter at intervals of 4-10 s and were based on experimenter assurance that the mouse was properly positioned within the tube. In a small percentage of cases (<5%), individual mdx mice failed to perform a sufficient number (approximately 15) of forward pulling maneuvers within the 5 min recording period. Results from these WBT sessions were not included in the analyses.

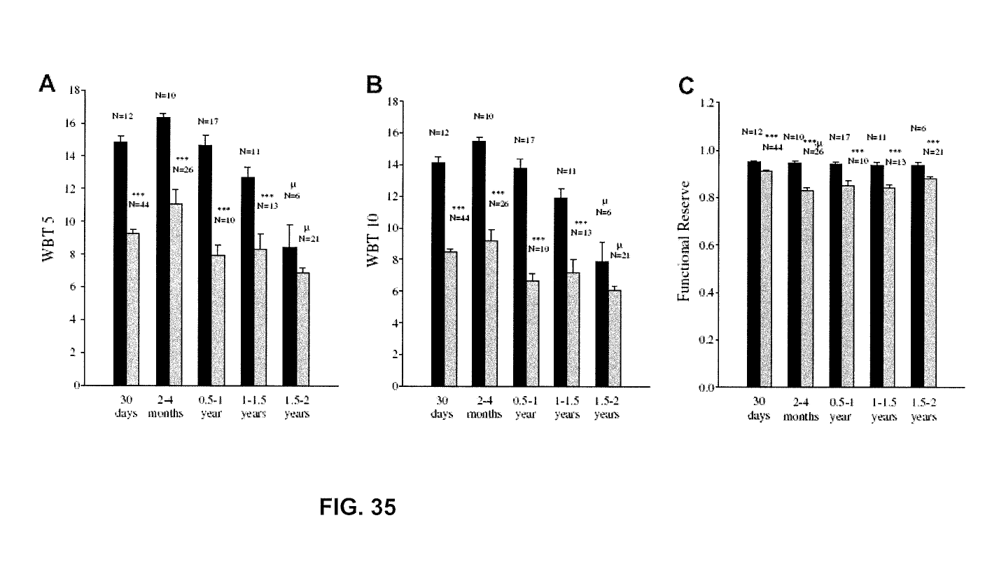

FIG. 34 shows portions of individual records of forward pulling tensions (FPTs) which illustrate the top 10 FPTs exerted during typical WBT recording sessions. The records were obtained from (A) a 7 month nondystrophic (C57Bl10SnJ) male (Wt., 38 g), (B) a 6 month mdx male (32 g) treated daily with vehicle (HEPES Ringer) and (C) a 6 month mdx male (34 g) treated for 30 consecutive days with 50 mg/kg PDTC. The ranked forward pulling tensions for each record are indicated above each tracing (1-10). The average of the top 10 FPTs divided by the body weight is the WBT10. The WBT 5 represents the average of the top 5 FPTs divided by the body weight and the FR equals the WBT10/WBT5. The PDP is obtained by determining the negative slope of the decline in normalized FPT for the top 10 FPTs as shown in FIG. 35. Calibrations in A, B, and C indicate 65 g (vertical) and 2 s (horizontal).

The individual FPTs obtained within each WBT recording session were ordered from largest to smallest in order to identify the top 10 and top 5 FPTs (numbers 1 through 10 in FIG. 34). Since approximately 15 and 20 FPTs were obtained from each session and since only data from the top 10 FPTs were used in the analyses, in effect, each mouse was given approximately 1.5-2 opportunities to manifest pulling strength for each of the top 10 FPTs that were ultimately utilized.

Portions of records obtained from nondystrophic mice, and from mdx mice treated either with daily vehicle or PDTC injections, show the top 10 FPTs (in gms) obtained during a typical WBT recording session, and indicate that the maximal FPT exerted by each mouse may occur at any particular time during the recording session (FIG. 34). For example, the 7 month nondystrophic mouse represented in FIG. 34A exerted a maximal FPT (FPT₁, number 1 in FIG. 34A) of approximately 490 g at roughly the midpoint of the record in FIG. 34A. An examination of the ordered FPTs during the session (FIG. 34A) indicates that the tenth highest FPT exerted by this mouse (i.e., number 10) was approximately 83% of FPT₁ (FIG. 34A). In contrast, the 6 month mdx male represented in FIG. 34B produced a maximal FPT of approximately 180 g (number 1 in FIG. 34B) and had an FPT₁₀ that was equal to only 36% of the FPT₁ value. The littermate mdx male treated with 50 mg/kg PDTC for 30 days exerted a maximum FPT of approximately 310 g, and had an FPT₁₀ that was approximately 77% of the FPT₁ (FIG. 34C). For each WBT recording session, four parameters were obtained: the average of the top 5 FPTs divided by body weight (WBT5); the average of the top 10 FPTs divided by body weight (WBT10); the WBT10/WBT5 (FR); and the negative slope of the decline in normalized FPT over the top 10 FPTs (PDP).

### WBT5, WBT10, and FR are Significantly Reduced Throughout the Lifespan of mdx Mice

FIG. 35 shows that Mdx mice exhibit significant deficits in FPT that begin at 30 days of age and continue throughout the mdx lifespan. (A) WBT5 measures indicate a significant (μ; p<0.05; ANOVA) peak in FPT generation at approximately 3 months (2-4 months) in both nondystrophic (black histobars) and mdx (grey histobars) populations. The WBT5 from the mdx population was substantially and significantly (***p<0.001) decreased at all age intervals with the exception of the aging population (1.5-2 years). (B) WBT10 values indicate similar age-related patterns (μ; p<0.05) in both the nondystrophic (black) and mdx (grey) population with significant (***p<0.001) deficits in the mdx population. (C) The FR of nondystrophic mice (black bars) is independent of age and remains at approximately 0.94-0.95 throughout the lifespan of the nondystrophic mouse. The FR of mdx mice is significantly lower than the corresponding nondystrophic values at all age intervals (***p<0.001) but undergoes a significant age-related decrease between 30 days and 2-4 months of age (μ, p<0.05). In the older mdx populations the FR remains constant at approximately 0.83-0.88. Means and standard errors are shown.

Both nondystrophic and mdx mice exhibited a developmental peak in WBT5 and WBT10 at approximately 3 months (2-4 months) that was followed by a subsequent decline as the animals aged (FIGS. 35A, B; Kruskal-Wallis ANOVA; p<0.001). In the nondystrophic mice, the WBT5 and WBT10 values significantly (Dunn's method, p<0.05) declined to approximately 67% and 50% of the peak value at 1.25 years and 1.75 years, respectively. Less substantial age-related declines (Dunn's method; p<0.05) were observed in the mdx population.

Mdx mice exhibited significantly (p<0.001, t-test) and substantially (about 60-62% of nondystrophic values) reduced WBT5 and WBT10 values as early as 30 days after birth (FIGS. 35A, B). The WBT5 and WBT10 values obtained from mdx mice remained significantly (p<0.001, t-test or Mann-Whitney rank sum test) reduced at 2-4 months, 6-12 mo, and 1-1.5 years. At 1.5-2 years of age, the values from the nondystrophic mice converged to approximate the reduced WBT values of the mdx population (FIGS. 35A, B).

The WBT10 divided by the WBT5 (FR) provides a measure of the consistency of tension generation over the top 10 FPTs for each mouse. Nondystrophic mice exhibited FR values of approximately 0.94-0.95 throughout their lifespan. The FR of mdx mice at 30 days (0.91) was significantly lower than the corresponding value from the nondystrophic population, and remained significantly (p<0.001) reduced throughout the lifespan of the mdx mouse (FIG. 35C; cf Carlson and Makiejus (1990)). In contrast to the population of nondystrophic mice, mdx mice exhibited a significant (Kruskal-Wallis ANOVA; p<0.001) age-related decrease in FR from 0.91 at 30 days to a significant low of 0.83 at 2-4 months (Dunn's method, p<0.05; FIG. 35C).

In summary, the results indicate that mdx mice exhibit significant whole body muscle weakness characterized by WBT5 and WBT10 values that were between 53% and 67% of the corresponding nondystrophic values at 1 month to 1.5 years. In general, the percent decrease in the WBT10 value exceeded the decrease in WBT5 by 5-8% leading to a significant reduction in the FR of the mdx population. In contrast to nondystrophic mice, the mdx mice exhibited a significant age-related decline in FR between the ages of 30 days and 3 months (FIG. 35A-C).

### Mdx Mice Exhibit Enhanced Weakening During a Typical WBT Recording Session

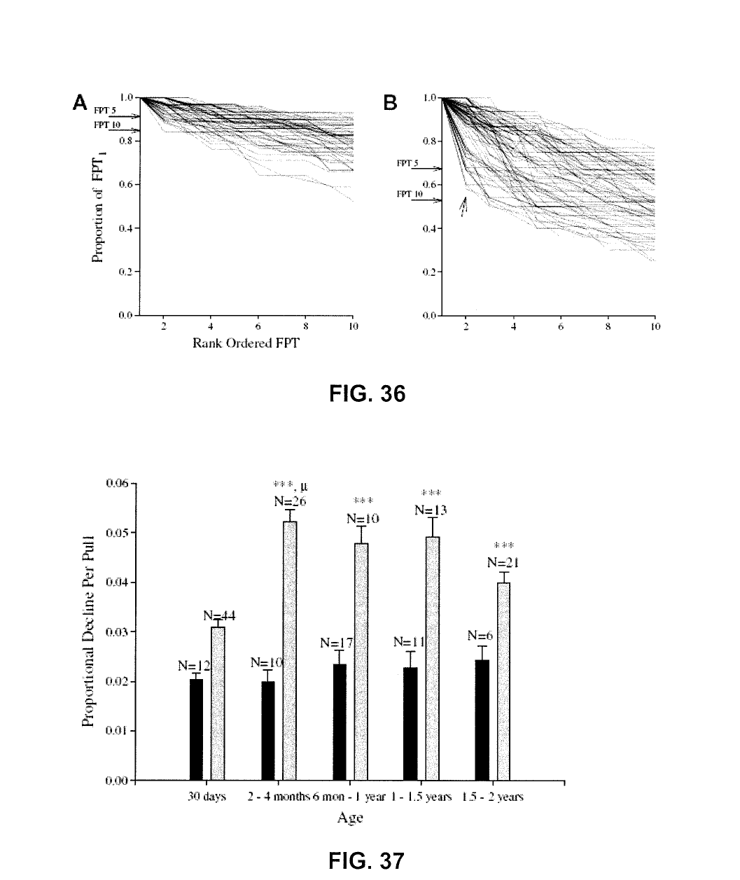

The reduced FR characteristic of the mdx population suggests that mdx mice exhibit enhanced weakening during the performance of a forward pulling maneuver. FIG. 36 summarizes the rank-ordered FPTs obtained from adult nondystrophic (FIG. 36A) and mdx (FIG. 36B) mice. More particularly, FIG. 36 shows that examination of the decline in FPT over the top 10 FPTs indicates that mdx mice exhibit a greater proportional decline in FPT per maximal forward pulling maneuver than nondystrophic mice. The ordinate shows the FPTs for rank-ordered pulls 1 through 10 normalized to the top FPT (FPT₁) observed during the WBT recording session (See e.g., FIG. 34). (A) Results from several adult nondystrophic mice (>3 months) indicate that the 5th and 10th largest FPTs (FPT5, FPT10; horizontal arrows) are on the average approximately 0.91 and 0.85 of the largest FPT. (B) Corresponding results from adult mdx mice indicate that the 5th and 10th largest FPTs are approximately 0.67 and 0.53 of the largest FPT (horizontal arrows). Oblique arrow indicates individual records exhibiting relatively large reductions in the second highest FPT (i.e., FPT2). The average FPT₅ and FPT₁₀ values for the nondystrophic population were approximately 0.91 and 0.85 of the FPT₁ value, respectively (FIG. 36A). In contrast, the FPT₅ and FPT₁₀ values for the mdx population were 0.67 and 0.53 of the FPT₁ value (FIG. 36B).

To provide a direct quantitative measure of the decline in rank-ordered FPTs, linear regressions of the normalized FPT (i.e., FPTₓ/FPT₁) were performed over the top 10 FPTs of each recording session. Although this approach may slightly underestimate the decline per pull in those cases that exhibited more substantial declines between FPT₁ and FPT₂ (e.g. arrow, FIG. 36B), it nevertheless provides a simple and reproducible determination of the average decline in FPT per maximal forward pulling effort (PDP).

The PDP values obtained from mdx mice were significantly (p<0.001) higher than nondystrophic values in all age categories (FIG. 37). FIG. 37 shows that Mdx mice exhibit significantly larger PDP values than age-matched nondystrophic mice. The average PDP was significantly (***p<0.001) elevated in the mdx population beginning at 30 days of age and extending throughout the mdx lifespan. The average PDP for the nondystrophic population remained at approximately 0.02 throughout the lifespan. In contrast, the mdx population exhibited a significant (μ, p<0.05) age-related increase in the PDP between 30 days and 2-4 months of age. Means and standard errors are shown.

Nondystrophic mice exhibited an average PDP of approximately 0.02 indicating a 2% decline in FPT per pull over the top 10 efforts. The PDP for nondystrophic mice did not vary with age, while the PDP for the mdx population increased significantly (p<0.001) between 30 days and 3 months. At 30 days of age, mdx mice had an average PDP of about 0.03 (3% decline per pull), while at 3 months the PDP of mdx mice (0.052) was greater than two times the corresponding value in the nondystrophic population (0.02). The PDP for the mdx population remained elevated at approximately 0.05 between 2 months and 1.5 years and then declined to approximately 0.04 in the aging population (FIG. 37). These results are consistent with the age-dependent reduction in FR seen in the 3 month mdx population (FIG. 35C) and indicate that mdx mice exhibit a greater proportional weakening following a “best effort” forward pulling maneuver than nondystrophic mice. This deficit significantly worsens between 30 days and 3 months after birth (FIGS. 35C and 37).

Daily Administration of UDCA Produces More Substantial Improvement in WBT than PDTC

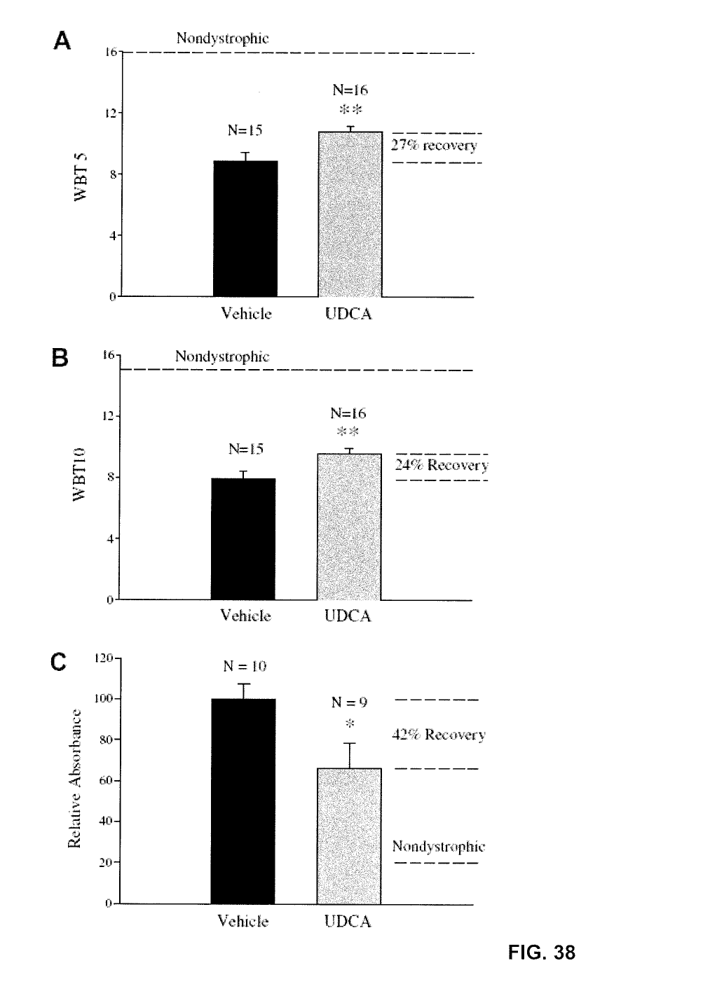

As shown in Examples 5-7 above, daily PDTC treatment produced moderate increases in WBT that were associated with a reduction of the characteristic weakening that occurred following a forward pulling maneuver. To assess the therapeutic effects of another NFkB inhibitor, namely, UDCA, experiments were performed to compare the effects of PDTC and UDCA on muscular dystrophy subjects. UDCA is an NF-κB inhibitor that interacts with the glucocorticoid receptor and inhibits nuclear transactivation of p65 (Miura et al., 2001). Treatment (40 mg/kg, ip, daily) was initiated at 30 days of age and continued for another 30 days. WBT determinations obtained at the end of the treatment period from the UDCA treated mice were significantly (p<0.01) higher than those obtained from the age-matched vehicle-treated mice (FIGS. 38A, B).

FIG. 38 shows that long term treatment of young adult mdx mice with UDCA increases WBT5 (A), WBT10 (B), and reduces nuclear p65 activation (C) in the mdx costal diaphragm. Horizontal dashed lines indicate average levels observed in nondystrophic, vehicle, and UDCA treated preparations as indicated. In each case, percent recovery is determined by dividing the mean improvement observed in the UDCA treated preparation (UDCA-vehicle) from the deficit observed in the vehicle-treated preparation (nondystrophic-vehicle mdx). Mdx mice were treated for 30 days beginning at 1 month of age. Horizontal dashed lines in (A) and (B) representing nondystrophic values were obtained from a cohort of nondystrophic mice at 2 months of age. (C) shows the results of nuclear p65 activation assays using the Trans AM ELISA-based method for determining relative amounts of p65 bound to the NF-κB consensus sequence. The results were obtained from two separate groups of vehicle and UDCA treated preparations that were assayed using two different 96 well plates. For each plate, the absorbance for each sample was normalized to the average absorbance determined for the vehicle-treated preparations on that plate. The results indicate that the UDCA treated preparations exhibited a mean absorbance that was approximately 66% of the absorbance in the vehicle-treated preparations. The approximate level for the nondystrophic preparations was estimated from parallel studies indicating that the absorbance in nuclear fractions from adult mdx costal diaphragm is approximately 5-fold higher than that observed in nondystrophic costal diaphragm. Millman et al., 2007. N is the number of mice and preparations in each condition. Shown are the means and standard errors. ** and ** indicate p<0.05 and p<0.01, respectively, between vehicle and UDCA treated preparations. WBT5 (FIG. 38A) and WBT10 values in the UDCA treated mdx mice were increased by about 21% above the levels observed in the vehicle-treated mice corresponding to a recovery of 27% and 24%, respectively, of the dystrophic deficit observed in WBT5 and WBT10. Since the increase in WBT10 and WBT5 induced by UDCA treatment were nearly identical, the drug had no influence on either FR or PDP.

### Effects of PDTC and UDCA Treatment on Nuclear p65 Activation in the Costal Diaphragm

Although previous studies had shown that long term treatment with PDTC effectively reduced nuclear NF-κB activation in mdx quadriceps (Messina et al. (2006)), others showed that long term treatment with the IκB-α kinase (IKK) inhibitor curcumin did not reduce nuclear NF-κB activation in the mdx costal diaphragm Durham et al. (2006). Therefore, the effects of long term treatment with PDTC and UDCA on nuclear p65 activation were examined using the mdx costal diaphragm which exhibits a larger increase in p65 nuclear activation than either the mdx gastrocnemius or triangularis sterni muscle. Millman et al. (2007). Parallel studies using mature adult mdx mice indicated that a 30 day PDTC treatment (50 mg/kg/day, ip) did not significantly reduce nuclear p65 activation in the costal diaphragm (23% reduction, N=4 vehicle-treated, N=3 PDTC-treated, p=0.4). Similarly, PDTC treatment of a group of young adult mdx mice for a period of 60 days beginning at 30 days of age had no effect on nuclear p65 activation in the mdx costal diaphragm (N=5 vehicle-treated, N=5 PDTC-treated). These results are at variance with the effects of long term treatment with PDTC on nuclear NF-κB activation in the mdx quadriceps muscle (Messina et al., 2006) and suggest that the very elevated levels of nuclear p65 activation seen in the mdx costal diaphragm are more resistant to treatment with NF-κB inhibitors than limb muscles which exhibit smaller dystrophic increases in nuclear p65 activation. Millman et al. (2007).

In contrast to the results with PDTC, however, 30 day treatment with UDCA produced a significant (p<0.05) 34% reduction in nuclear p65 activation in the mdx costal diaphragm (FIG. 38C) corresponding to a 42% recovery towards the values observed in mature nondystrophic preparations. Singh et at (2009). These results indicate that the larger improvement in the WBT parameters observed in the UDCA treated mdx mice was associated with superior efficacy in reducing nuclear p65 activation during long term treatment.

### Example 15

### Direct Comparisons Between the Cellular Expression of Each of the Primary Signaling Components of the NF-κB Pathway in mdx and Nondystrophic Muscle

Studies were conducted to examine the expression of major NF-κB signaling molecules in muscle tissues isolated from nondystrophic (i.e. normal) or mdx mice in order to provide new information relevant to the development of improved methods for decreasing NF-κB activation in dystrophic muscle (Singh et al., 2009). The protein p65 is an important component (or subunit) of the NF-κB dimer and contains the protein domain that is responsible for transactivation of the NF-κB promoter and inducing the expression of NF-κB dependent genes. More specifically, these results show that the elevated nuclear activation of NF-κB in dystrophic muscle is secondary to the increased cellular expression of most of the components of the NF-κB pathway in skeletal muscle, such as p65. These results further suggest that the increased NF-κB activation in dystrophic muscle is likely not caused solely by elevated IκB-α kinase (IKK) activity. In summary, direct comparisons between the cellular expression of each of the primary signaling components of the NF-κB pathway in mdx and nondystrophic muscle indicate that agents which inhibit p65 transactivation or p65 expression have enhanced utility in depressing nuclear NF-κB activation in dystrophic muscle. These studies are described in details below.

### Muscle Preparations

The gastrocnemius muscle preparation consisted of the medial and lateral heads along with the associated plantaris muscle. The two costal hemi-diaphragms were also obtained along with the TS muscles. Age-matched nondystrophic (C57Bl10-SnJ) and mdx mice (C57Bl10- mdx) mice were euthanized by cervical dislocation following exposure to CO₂ or following Na+ pentobarbital anesthesia using protocols approved by the Institutional Animal Care and Use Committee (IACUC). After removing the muscles in a HEPES Ringer saline solution (147.5 mM NaCl, 5 mM KCl, 2 mM CaCl2, 11 mM glucose, 5 mM HEPES, pH 7.35), the preparations were blotted, flash-frozen and stored at −80° C. for subsequent analysis. All animal experiments were approved by the IACUC in accordance with NIH guidelines.

### MG132 Administration

The influence of the proteasomal inhibitor MG132 on the cytosolic expression of IκB-α and Pi-IκB-α was determined by administering 5 month nondystrophic and mdx mice MG132 (1 mg/kg, intraperitoneal) or vehicle (DMSO) in vivo 2 h prior to euthanization and isolation of the diaphragm. The individual intact costal diaphragms from the MG132 group were then exposed to MG132 (50 mM in HEPES Ringer) in vitro for another 2 h while the costal diaphragms from the vehicle group were exposed to HEPES Ringer and vehicle. This procedure of combining in vivo and in vitro exposure to MG was used to maximize inhibition of proteasomal degradation and facilitate the cytosolic accumulation of Pi-IκB-α and IκB-α in the MG132 treated preparations.

### Muscle Extracts

Whole cell lysates (WCL) were prepared using a Dounce homogenizer and isotonic lysis buffer (WCLB: 20 mM Tris-HCl, 150 mM NaCl, 1 mM Na₂ EDTA, 1 mM EGTA, 1% Triton, 2.5 mM sodium pyrophosphate, 1 mM beta-glycerophosphate, 1 mM Na3VO4, 1 mM phenylmethylsulfonyl fluoride, 1 mM dithiothreitol, 4 μl/ml protease inhibitor cocktail, 10 μl/ml phosphate inhibitor, pH 7.5) at a concentration of 15 μl per mg tissue. Homogenized tissue was then subjected to two freeze/thaw cycles (5 min freezing on dry ice) and subsequently vortexed for approximately 10 s before centrifugation (6 min, 13,000 rpm, 4° C.). The supernatant containing the whole cell lysate was removed and stored at −80° C.

Cytosolic and nuclear extracts were obtained by homogenizing individual muscles at a concentration of 1 mg wet weight per 18 μl of low salt lysis buffer (LSLB: 10 mM HEPES, 10 mM KCl, 1.5 mM MgCl₂, 0.1 mM EDTA, 0.1 mM EGTA, 1 mM dithiothreitol, 1.0 mg/ml benzamidine, 0.5 mM phenylmethylsulfonyl fluoride, 4.0 μl/ml protease inhibitor cocktail Sigma #8340, 10 μl/ml phosphatase inhibitor Sigma #P2850, pH 7.9. See Kumar et al. (2003); Carlson et al. (2005)). The homogenized muscle was maintained on dry ice for at least 10 min, and subjected to two subsequent cycles of freeze-thaw lysis using dry ice (5 min freeze, thaw at room temperature) before centrifugation (13,000 rpm, 20 s, 4° C.) and removal of the supernatant (cytosolic extract; stored at −80° C.). Pellets were subsequently washed twice (50 μl of LSLB) and resuspended in ice-cold high-salt lysis buffer (HSLB: 20 mM HEPES (pH 7.9), 420 mM NaCl, 1 mM EDTA, 1 mM EGTA, 25% glycerol, 1 mM dithiothreitol, 0.5 mM phenylmethylsulfonyl fluoride, 1.0 mg/ml benzamidine, 4 μl/ml protease inhibitor cocktail, 10 μl/ml phosphate inhibitor; 4 μl/mg wet 5 min intervals and centrifuged (13,000 rpm, 6 min, 4° C.). The supernatant (nuclear extract) was stored at −80° C. Protein concentration was determined using the Bradford assay (Bio-Rad 500-0006) in a standard 96 well plate.

### DNA Binding Activity

DNA binding was determined using the TransAM™NF-κB p65 assay kit (Active Motif, Carlsbad, Calif.) in accordance with the manufacturer's instructions. Sample absorbance was directly compared between individual muscle preparations on individual plates and normalized to a standard amount of positive control (2.5 μg Jurkat nuclear extract).

### Western Blotting

Muscle proteins and immunoprecipitates (20 μg) were separated by SDS-PAGE under reducing conditions and transferred to PVDF membranes (Bio-Rad). Membranes were blocked with 5% non-fat dehydrated milk in Tris buffered saline containing Tween 20 (TBST; 25 mM Tris, pH 7.4, 137 mM NaCl, 2.7 mM KCl, 0.1% Tween20) for 1 h at room temperature. Primary antibodies were incubated with 5% BSA in TBST overnight at 4° C. Secondary antibody horseradish peroxidase (HRP) conjugate anti-rabbit IgG (Jackson Laboratory) was incubated with 5% non-fat dehydrated milk in TBST for 1 h at room temperature. The immunoblot detection was performed using the ECL detection system (Amersham, UK) according to the instructions of the manufacturer. After obtaining the desired immunoblot, the PVDF membrane was stripped for 30 min at 50° C. and re-probed with a second type of primary antibody. Equal loading of proteins for cytosolic and whole cell extracts was evaluated by developing a blot for GAPDH. The transcription factor oct1 was used as a loading check for nuclear extracts. Densitometric analysis was performed using Image J, Sigma Stat and Sigma Plot.

### Antibodies and Reagents

The anti-p-p65^{Ser536}, p65, Rel B, p-IKKα^{[ser180]/[ser181]}, IKKβ, IKKα, p-Iκβ-α^{ser32}, IκB-α, p100/52, Pi-Akt, Akt, and GAPDH antibodies were purchased from Cell Signaling Technology (Boston, Mass.). The antibodies against p105/50 and TRAF-3 were purchased from Santa Cruz Biotechnology Inc. (Santa Cruz, Calif.). The TransAM NF-kappa B p65 ELISA kit #40096 was used to determine p65 binding in nuclear extracts (Active Motif, Carlsbad, Calif.). The cell lysis buffer used to obtain whole cell lysates was purchased from Cell Signaling Technology (#9803). The protease inhibitor cocktail (cat #P8340), phosphatase inhibitor (P2850) cocktail, phenylmethanesulfonyl fluoride (P7626), dl-dithiothreitol (D0632), benzamidine hydrochloride hydrate (B6506), and proteasomal inhibitor MG132 (Z-leu-leu-leu-al; C2211) were purchased from Sigma-Aldrich (St. Louis, Mo.).

### Immunoprecipitation

Muscle cell protein lysates were diluted to 1 μg protein/μl in RIPA lysis buffer (Santa Cruz Biotechnology, sc-24948) and were incubated with 10 μg primary antibody and resuspended in 10 μl volume of protein A/G PLUS-Agarose (Santa Cruz Biotechnology, sc-24948) at 40° C. with gentle shaking (overnight). Immune complexes were collected by centrifugation at 13,000 rpm for 10 min, and washed four times with 100 μl RIPA buffer.

### Statistical Analyses

Densitometric evaluation of protein levels was conducted by dividing the density of the protein of interest by the appropriate protein used to check equal loading between lanes (GAPDH for cytosolic and whole cell lysates; oct 1 for nuclear extracts). Evaluation of the phosphorylation status of a particular protein was determined by dividing the density of the phosphorylated protein (e.g. P-Akt) by the total amount of the relevant protein present (e.g., Akt). Comparisons were made between age (3 to 5 months) and gender-matched (equal distribution of males and females per group) groups of mdx (C57Bl10-mdx) and nondystrophic (C57Bl10SnJ) mice. Direct comparisons between groups of nondystrophic and mdx mice were evaluated using t tests or Mann-Whitney Rank Sums tests (0.05 level of significance; Sigmaplot 9.0 and Sigmastat v 3.0). Muscle-specific differences were evaluated using one way ANOVA or Kruskall-Wallis one way ANOVA on ranks followed by direct comparisons using Tukey's test or Dunn's test.

### Results

### Nuclear p65 Activation

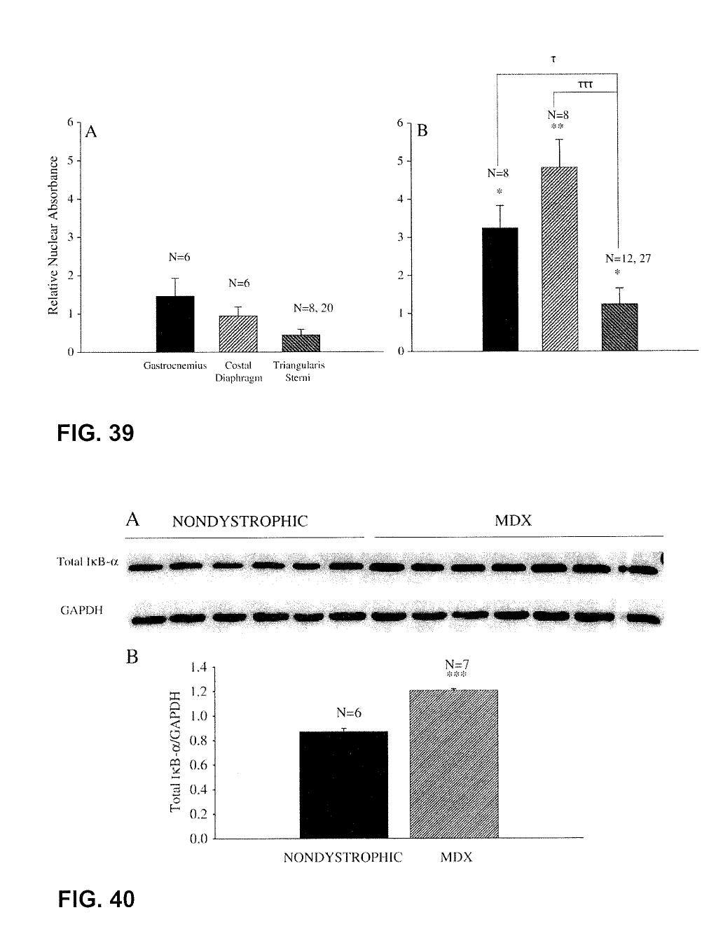

Nuclear p65 activation was significantly increased in the limb and respiratory musculature of the mdx mouse with the largest proportional increase in the costal diaphragm (5.1 fold increase above nondystrophic; p=0.01) and the smallest in the gastrocnemius muscle (2.2 fold increase, p<0.05; FIG. 39). FIG. 39 shows that nuclear extracts from limb and respiratory musculature exhibit significant increases in nuclear p65 activation. Panel A shows the nuclear absorbances obtained using the Trans AM ELISA based assay (Active Motif) for activated p65 from nondystrophic nuclear extracts of gastrocnemius, costal diaphragm, and TS muscles are presented relative to a positive control containing a standard amount of nuclear protein (2.5 μg) obtained from Jurkat cells. The positive control was run in parallel to specific muscle (e.g. gastrocnemius, costal diaphragm, or TS) nuclear (3 μg) samples from age-matched (4 to 6 month old, male) nondystrophic and mdx mice. Panel B shows the nuclear absorbances representing activated p65 from nuclear extracts of mdx gastrocnemius, costal diaphragm and TS muscles. *, and ** indicate p<0.05 and p=0.01 in comparison to corresponding nondystrophic muscle nuclear extracts. T, and TTT indicate p<0.05 and p<0.001 between indicated mdx muscle samples (ANOVA followed by Tukey test). The largest proportional increase above nondystrophic levels for nuclear p65 activation was observed for the costal diaphragm (**). For the gastrocnemius and costal diaphragm muscle preparations, N represents the number of mice (and corresponding muscle preparations with 1 preparation per mouse). For the TS muscle preparations, N represents the number of samples, and the number of mice and corresponding muscle preparations (both left and right TS muscles) that were pooled to obtain sufficient nuclear protein for the assay. Shown are the means and standard errors.

Although nuclear p65 activation was not significantly different between the 3 nondystrophic muscles examined (FIG. 39A), a one way ANOVA indicated a muscle-specific difference in the activation observed in the mdx muscles (p<0.001; FIG. 39B). The largest nuclear p65 activation was observed in the mdx costal diaphragm and the smallest in the mdx TS muscle. In the mdx mouse, the nuclear extracts from both the gastrocnemius (p<0.05; Tukey) and the costal diaphragm (p<0.001; Tukey) exhibited significantly larger activated p65 levels than the TS muscle (FIG. 39B). These results indicate that the NF-κB pathway is dysregulated in the mdx musculature, with the largest proportional increase in nuclear p65 activation occurring in the costal diaphragm.

Expression of IκB-α in Whole Cell Lysates from mdx and Nondystrophic Costal Diaphragm

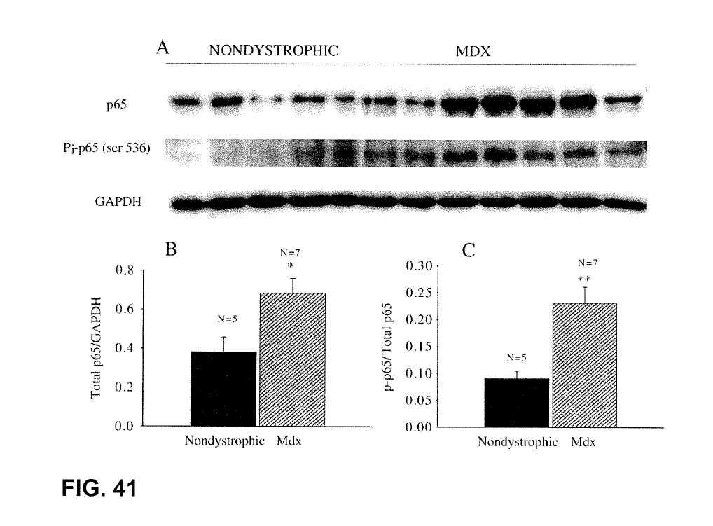

Since the costal diaphragm exhibited the largest proportional increase in nuclear p65 activation, it was used to examine the expression of the major signaling components in the classical and alternative NF-κB pathways. IκB-α is an NF-κB gene product, and would be expected to accumulate with an increase in nuclear p65 activation. Siebenlist et al. (1994). However, increased IKK activity in mdx cytosol would enhance the proteasomal degradation of IκB-α and reduce levels of the protein in cytosolic extracts and whole cell lysates. See Kumar et al. (2003); and Acharyya et al. (2007). Densitometric evaluation of whole cell lysates using GAPDH as a loading control indicated that total cellular IκB-α was significantly (p<0.001) increased by 39% in the mdx costal diaphragm (FIG. 40). FIG. 40 shows Increases in whole cell expression of IκB-α in mdx costal diaphragm. Panel A shows Western blots of IκB-α (top row) and GAPDH (lower row) for whole cell lysates from 5 month old nondystrophic (left) and mdx (right) costal diaphragms. Panel B shows densitometric evaluation of total IκB-α relative to GAPDH for nondystrophic (black histobar) and mdx (gray cross-hatched histobar) costal diaphragm whole cell lysates. *** indicates p<0.001 in comparison to nondystrophic diaphragm. N refers to the number of mice and costal diaphragm preparations. Shown are the means and standard errors (B, C).

Expression of p65, p105, and p50 in mdx and Nondystrophic Costal Diaphragm

Total cellular expression of p65 was determined in whole cell lysates from age-matched (5 month) adult nondystrophic and mdx costal diaphragms. Whole cell extracts from mdx diaphragms exhibited p65 levels (normalized to GAPDH) that were 1.8 fold higher than those observed in nondystrophic costal diaphragm (p<0.05; FIG. 41 B). The amount of phosphorylated p65 was quite low in nondystrophic muscle but was apparent in the whole cell extracts from mdx diaphragm (FIG. 41A). The proportion of p65 in the phosphorylated state in the mdx diaphragm was increased 2.5 fold relative to what was observed in nondystrophic diaphragm (p<0.01; FIG. 41 C). FIG. 41 shows increases in the whole cell expression of p65 in mdx costal diaphragm. (A)Western blots of p65 (top row), Pi-p65 (ser 536; middle row), and GAPDH (lower row) for whole cell lysates from 3 month old nondystrophic (left) and mdx (right) costal diaphragms. (B) Densitometric evaluation of total p65 relative to GAPDH. (C) Proportion of phosphorylated p65. Black histobars—nondystrophic, gray cross—hatched histobars—mdx. * and ** indicate p<0.05 and p<0.01, respectively, in comparison to nondystrophic values. N refers to the number of mice and corresponding muscle preparations. Shown are the means and standard errors (B, C).

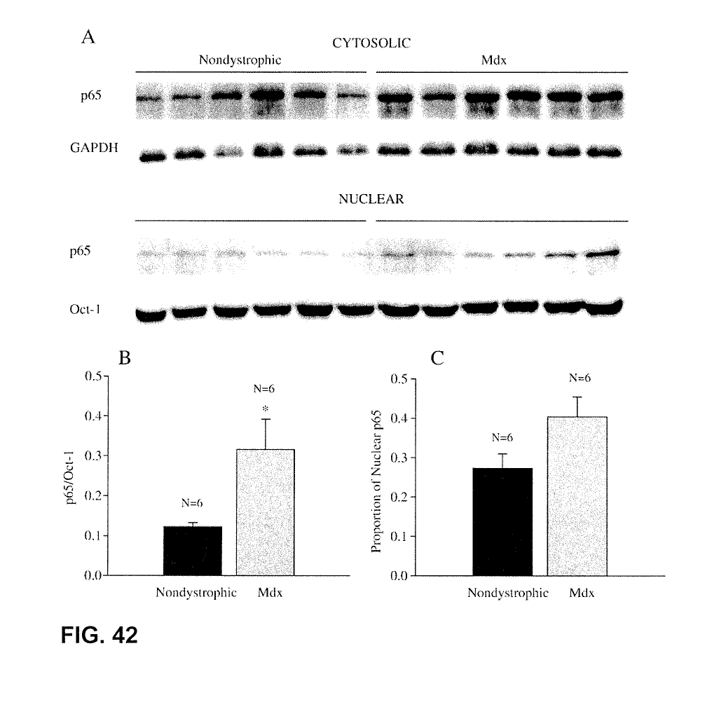

The results from nuclear extracts indicated that the absolute amount of p65 in the nuclear compartment was significantly (p<0.05) increased by 2.6 fold in the mdx costal diaphragm (FIGS. 42A, 42B). FIG. 42 shows increases in the cytosolic and nuclear expression of p65 in mdx costal diaphragm. (A)Western blots of p65 (top rows) and appropriate loading controls (GAPDH for cytosolic fractions, Oct-1 for nuclear extracts; bottom rows) for cytosolic and nuclear extracts obtained from 4 to 5 month old nondystrophic and mdx costal diaphragms. (B) Nuclear expression of p65 relative to Oct-1 as loading control. (C) Proportion of nuclear p65 determined using the density of cytosolic and nuclear p65 corrected for the appropriate loading control ((nuclear p65/Oct1)/((nuclear p65/Oct1)+(cytosolic p65/GAPDH))). Black histobars—nondystrophic; gray histobars—mdx. * indicates p<0.05 in comparison to nondystrophic levels. Shown are the means and standard errors. N is the number of mice and corresponding muscle preparations.

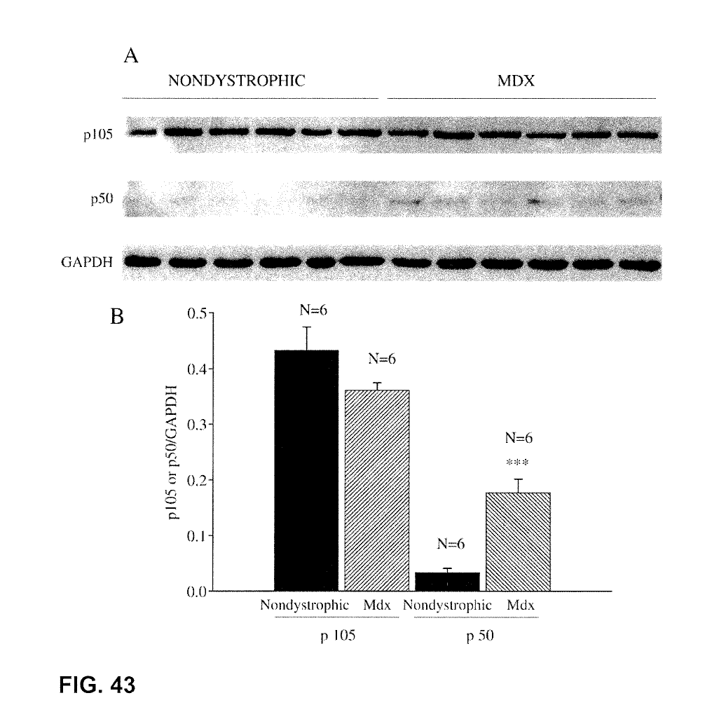

The percentage of p65 in the nuclear compartment (corrected for loading using GAPDH and Oct 1) was increased by 1.5 fold in the mdx costal diaphragm, an effect which failed to reach statistical significance (p=0.06; FIG. 42C). The expression of p50 in nondystrophic muscle was quite low relative to the expression of p105 (FIG. 43A). FIG. 43 shows increases in the whole cell expression of p50 in mdx costal diaphragm. (A)Western blots of p105 (top row), p50 (middle row), and GAPDH (bottom row) for whole cell lysates from 5 month old nondystrophic (left) and mdx (right) costal diaphragms. (B) Densitometric determination of p105 and p50 expression relative to GAPDH. *** indicates p<0.001 relative to nondystrophic preparations. Shown are the means and standard errors. N is the number of mice and corresponding costal diaphragm extracts. Whole cell extracts from mdx muscle exhibited p50 levels that were approximately 5.3 fold higher than those in nondystrophic muscle (p<0.001). Expression of the precursor p105 was reduced slightly (p>0.05) in mdx muscle in comparison to its expression in nondystrophic muscle (FIGS. 43A, B). These results indicate that the proportion of phosphorylated p65 and the expression of p65 and p50 are substantially elevated in the mdx diaphragm (FIGS. 41-43).

### Expression of the IKK Complex in mdx and Nondystrophic Costal Diaphragm

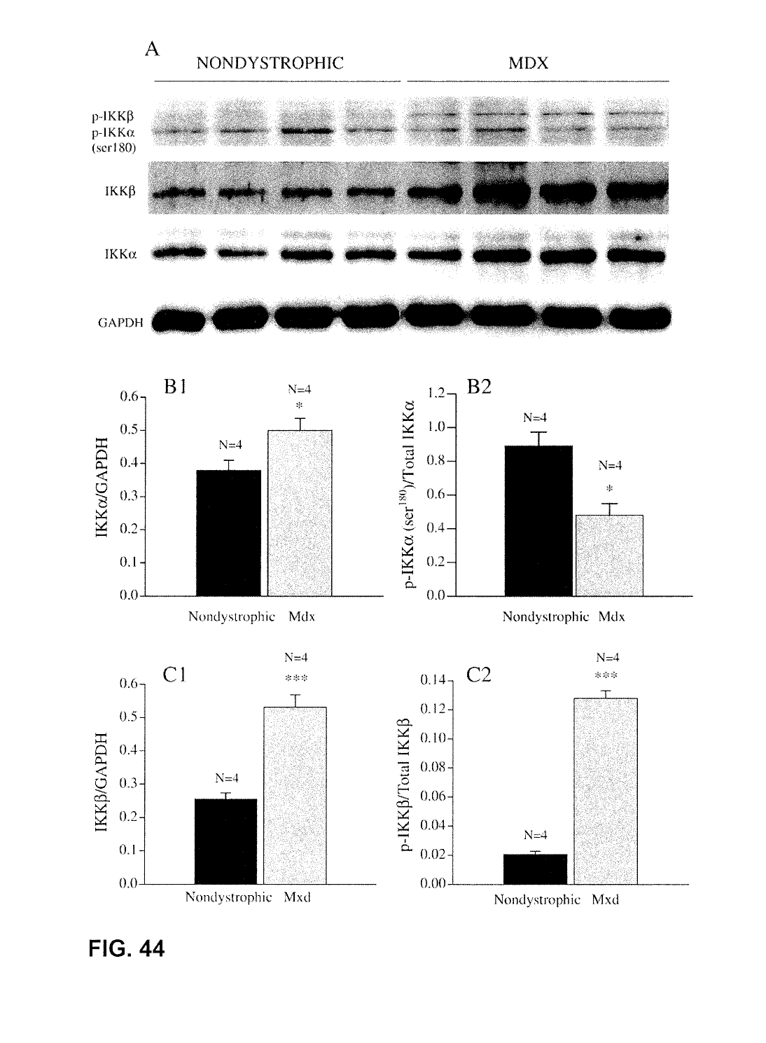

FIG. 44 shows alterations in the whole cell expression of IKKα and IKKβ in mdx costal diaphragm. (A)Western blots of phospho-IKKβ (ser 181) and phospho-IKKα (ser 180; top row), IKKβ (2nd row), IKKα (3rd row), and GAPDH (bottom row) for whole cell lysates from 5 month old nondystrophic (left) and mdx (right) costal diaphragms. (B1) Expression of IKKα relative to GAPDH. (B2) Proportion of phosphorylated IKKα. (C1) Expression of IKKβ relative to GAPDH. (C2) Proportion of phosphorylated IKKβ. * and *** indicate p<0.05 and p<0.001 in comparison to nondystrophic preparations. Shown are the means and standard errors, N is the number of mice and muscle preparations (B, C). The expression of IKKβ relative to GAPDH in whole cell lysates from 5 month old mdx costal diaphragm preparations was approximately double the level in age-matched nondystrophic diaphragm (p<0.001; FIGS. 44A and 44C1). In younger preparations (3 months), IKKβ expression was significantly (p<0.01) reduced to approximately 68% of nondystrophic levels in the mdx costal diaphragm. At 5 months, IKKα expression was increased by 1.3 fold (p<0.05; FIGS. 44A and B1). In another sample of mice aged between 3 and 5 months, IKKα expression relative to GAPDH was identical in nondystrophic and mdx costal diaphragms. These results suggest that the expression of IKKα and IKKβ in mdx costal diaphragm is a function of age and that significant increases in the expression of each of these subunits are not observed in younger adult preparations that may be undergoing more extensive muscle regeneration. However, by 5 months of age, the expression of both IKKα and IKKβ are significantly increased relative to nondystrophic levels (FIGS. 44A, 44B1 and 44C1).

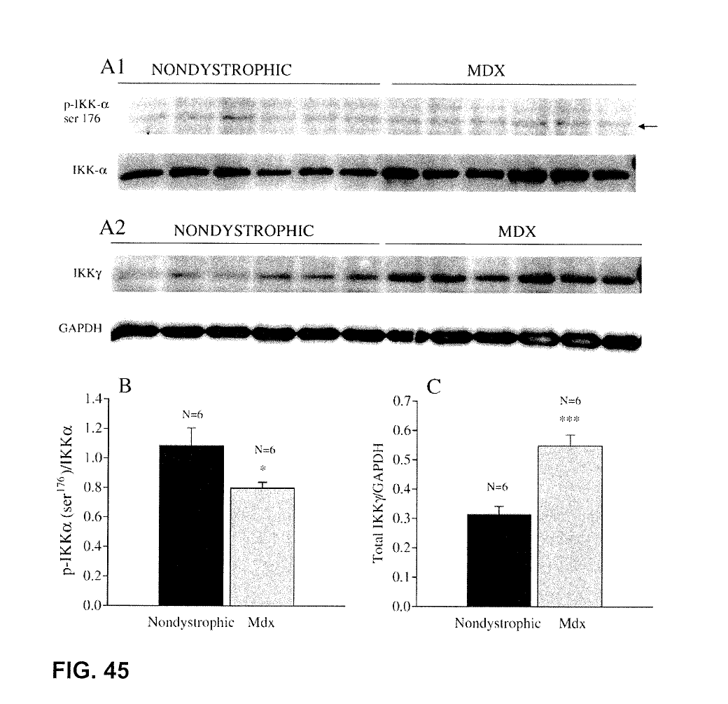

Consistent with these results, IKKγ expression in 5 month old mdx costal diaphragm was significantly increased to levels that were 1.75 fold higher than those observed in age-matched nondystrophic costal diaphragms (p<0.001; FIGS. 45A2 and 48C). FIG. 45 shows expression of phosphorylated IKKα (ser 176), total IKKα, and IKKγ in mdx costal diaphragm. (A1) Western blots of phospho-IKKα (p-IKKα, ser 176, top row, arrow-right side) and total IKKα (bottom row) in whole cell lysates from 5 month old nondystrophic (left) and mdx (right) costal diaphragms. (A2) Western blots of IKKγ (top) and GAPDH (bottom) in whole cell lysates from 5 month old nondystrophic (left) and mdx (right) costal diaphragms. (B) Proportion of IKKα phosphorylated at ser 176. (C) Densitometric evaluation of IKKγ expression. N is the number of mice and corresponding muscle preparations. * and *** indicate p=0.05 and p<0.001, respectively. Black histobars—nondystrophic, gray histobars—mdx.

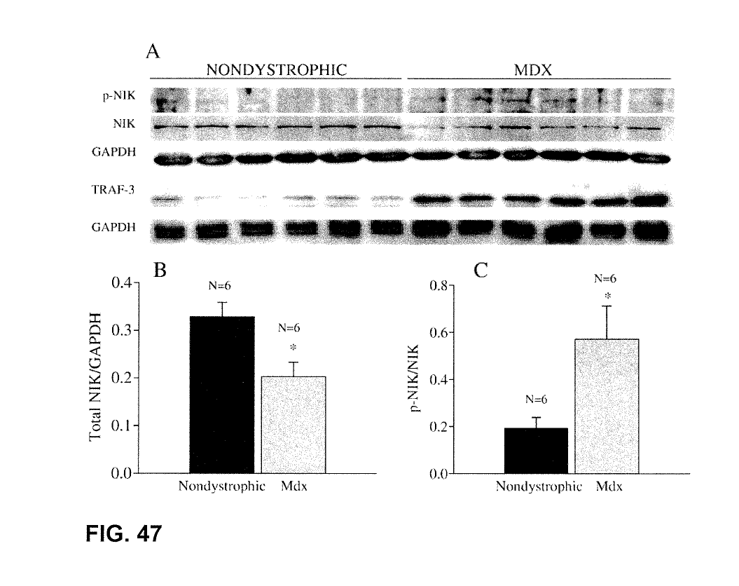

The proportion of total cellular IKKβ in the phosphorylated state in 5 month old mdx diaphragm whole cell lysates was 6 fold higher than that observed in nondystrophic diaphragm (p<0.001; FIGS. 44A and 47C2). In contrast, the proportions of IKKα phosphorylated at ser 180 (FIGS. 44A and 44B2) and at ser 176 (FIG. 45A1—arrow, B) were significantly (p<0.01) reduced in the mdx costal diaphragm.

Expression of RelB, p100, p52, NIK, and Phosphorylated NIK in mdx and Nondystrophic Costal Diaphragm

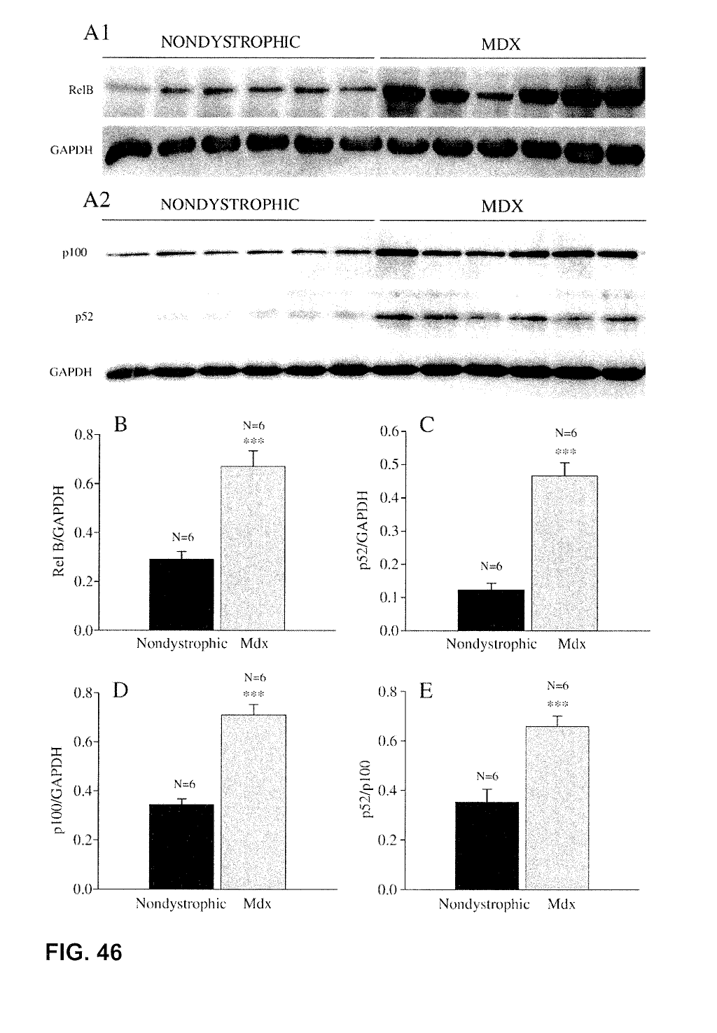

Rel B expression relative to the GAPDH loading control was increased by 2.3 fold in mdx compared to nondystrophic costal diaphragm (p<0.001; FIGS. 46A1 and 49B). FIG. 46 shows increases in the whole cell expression of alternative NF-κB pathway signaling components in the mdx costal diaphragm. (A1) Western blots of RelB and GAPDH and (A2) p100 (top row), p52 (middle row) and GAPDH (bottom) in whole cell lysates from 5 month old nondystrophic (left) and mdx (right) costal diaphragms. (B) through (D) show the expression of RelB (B), p52 (C), and p100 (D) relative to the GAPDH loading control. (E) shows the relative levels of expression for p52 and p100 (p52/p100). Black histobars—nondystrophic; gray histobars—mdx. N is the number of mice and corresponding muscle preparations. *** indicates p<0.001 in comparison to nondystrophic preparations.

Whole cell lysates from mdx costal diaphragm also exhibited significant increases in the expression of p100 (2 fold, p<0.001; FIGS. 46A2 and 46D) and p52 (3.8 fold, p<0.001; FIGS. 46A2 and 46C). The ratio of p52 expression to p100 expression was significantly (2 fold, p<0.001) increased, thus indicating enhanced processing of p100 in the mdx costal diaphragm (FIG. 46E).

Total cellular NIK expression relative to GAPDH was significantly (p<0.05) reduced by 40% in mdx diaphragm, but the proportion of phosphorylated NIK in mdx diaphragm was increased 3 fold (p<0.05) above the levels observed in the nondystrophic preparations (FIG. 47). FIG. 47 shows Alterations in the expression of NIK, phospho-NIK, and TRAF-3 in mdx costal diaphragms. (A)Western blots of phospho-NIK (p-NIK; Thr 559; top row), NIK (2nd row), GAPDH (3rd row) in whole cell lysates from 5 month old nondystrophic (left) and mdx (right) costal diaphragms. The whole cell lysates were run on a second gel to determine expression of TRAF 3 (4th row) relative to GAPDH (5th row). (B) Relative expression of NIK. (C) Proportion of phosphorylated NIK. * indicates p<0.05 in comparison to nondystrophic preparations. Shown are the means and standard errors, N refers to the number of mice and corresponding muscle preparations. The net effect was to produce no change in the level of phosphorylated NIK relative to GAPDH (p>0.05). Consistent with the negative influence of TRAF 3 on NIK expression, TRAF 3 expression was substantially elevated in mdx whole cell lysates (FIG. 47A). Hayden and Ghosh (2008).

The Effect of MG 132 on the Proportion of Phosphorylated IκB-α and the Expression of IκB-α in Cytosolic Extracts from Nondystrophic and mdx Costal Diaphragms

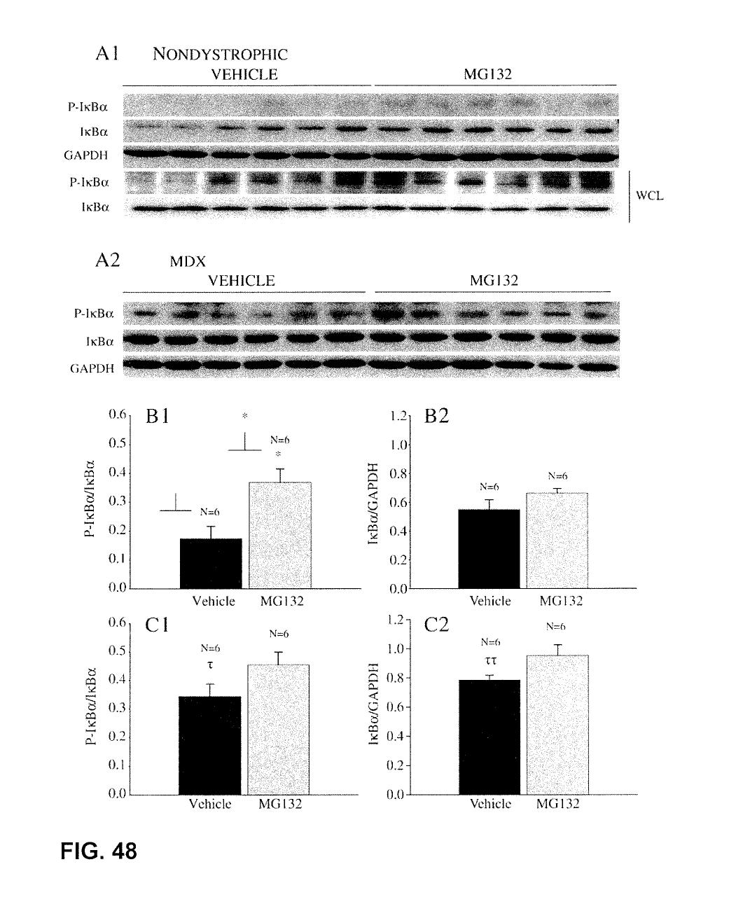

In contrast to the results from previous investigations, IκB-α expression (relative to GAPDH) in untreated mdx cytosol was significantly (p=0.01) increased by 1.4 fold over the levels observed in nondystrophic preparations (FIGS. 48A1—vehicle, A2—vehicle, B2—black histobar—nondystrophic, C2—black histobar—mdx). Kumar et al. (2003); and Acharyya (2007). Phosphorylated IκB-α was quite low in nondystrophic cytosol and in whole cell extracts (FIG. 48A1—vehicle).

FIG. 48 shows the influence of proteasomal inhibition on the cytosolic expression of IκB-α and phospho-IκB-α in nondystrophic and mdx costal diaphragm. (A1) Western blots of phospho-IκB-α (top row), IκB-α (2nd row), and GAPDH (3rd row) from cytosolic extracts of 5 month old nondystrophic mice pre-treated with MG132 (right side) or vehicle (left). Whole cell extracts (WCL) from these vehicle and MG132 treated nondystrophic diaphragms were also used to verify the expression of phospho-IκB-α (4th row) relative to total IκB-α (5^{th} row). (A2) Corresponding Western blots obtained from cytosolic extracts of mdx costal diaphragms from 5 month old mice. (B) The effect of MG132 treatment on the proportion of phosphorylated IκB-α (B1) and on the relative expression of IκB-α (B2) in the cytosol of nondystrophic costal diaphragms. Horizontal and vertical lines in B1 represent the mean and standard errors for the proportion of phosphorylated IκB-α in the whole cell extracts from vehicle and MG132 treated preparations. (C) The effect of MG132 treatment on the proportion of phosphorylated IκB-α (C1) and expression of IκB-α (C2) in the cytosol of mdx costal diaphragms. Black histobars—vehicle treated; gray histobars—MG132 treated. N is the number of mice and corresponding muscle preparations. * indicates p<0.05 in comparison to nondystrophic vehicle-treated preparations (B1). T (01) and TT (C2) indicate p<0.05 and p<0.01 in comparison to corresponding measures obtained from vehicle-treated nondystrophic preparations. Shown are the means and standard errors.

Consistent with the increased expression of IKK complex components and the increase in phosphorylated IKKβ in mdx whole cell lysates (FIG. 44), the amount of phosphorylated IκB-α and the proportion of phosphorylated IκB-α in the cytosol of mdx costal diaphragms was significantly higher (p<0.05) than that observed in the untreated nondystrophic diaphragms (FIGS. 48A1—vehicle, A2—vehicle, B1—black histobar—nondystrophic, C1—black histobar—mdx).

Treatment with the proteasomal inhibitor MG132 produced a significant (p<0.05) doubling of the proportion of phosphorylated IκB-α in both cytosol and whole cell extracts from nondystrophic diaphragm (FIGS. 48A1 and 48B1). However, MG132 did not significantly increase the proportion of phosphorylated IκB-α in cytosolic extracts from the mdx diaphragm(1.3 fold increase, pN 0.05; FIGS. 48A2 and 48C1). The expression of cytosolic IκB-α was not significantly increased by MG132 in either preparation (FIGS. 48B2 and 48C2). These results indicate that the untreated nondystrophic proteasome more effectively removes Pi-IκB-α from the cytosol than the untreated mdx proteasome.

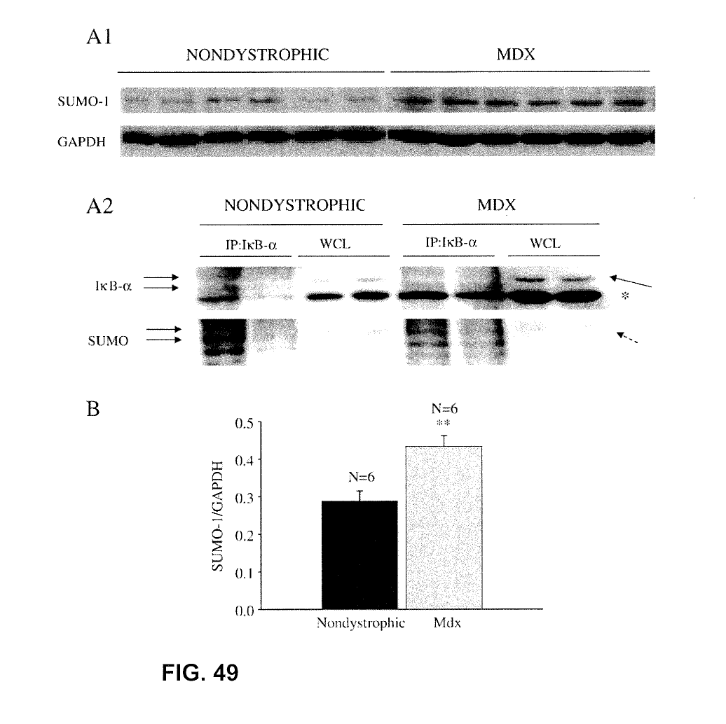

To examine whether cytosolic IκB-α may be protected from degradation in the mdx cytosol, the cytosolic expression of SUMO was determined in nondystrophic and mdx costal diaphragms. Densitometric evaluation indicated a significant (p<0.01) 1.5 fold increase in SUMO expression (relative to GAPDH) in the cytosol of mdx costal diaphragm (FIGS. 49A1 and 49B).

FIG. 49 shows SUMO expression and sumoylation of IκB-α in nondystrophic and mdx costal diaphragm. (A1) Western blots of SUMO-1 (top) and GAPDH (bottom row) in cytosolic extracts from 5 month old nondystrophic (left) and mdx (right) costal diaphragms. (A2) Western blots of whole cell lysates (WCL) and corresponding whole cell lysate immunoprecipitates of IκB-α (IP: IκB-α) obtained from 4 to 5 month old nondystrophic (left) and mdx (right side) costal diaphragms. Top row shows immunoblot for IκB-α and lower row immunoblot for SUMO. * on right side of top row indicates primary IκB-α band and arrows on left side indicate two bands that co-stain for IκB-α (top row) and SUMO (bottom row) in IκB-α immunoprecipitates. Angled solid arrow on right side indicates the presence of a minor band that is positive for IκB-α (top row) and SUMO (dashed angled arrow on right side, bottom row) in whole cell lysates. (B) Densitometric evaluation of total SUMO expression relative to GAPDH in cytosolic extracts from nondystrophic and mdx diaphragm (means and standard errors). Black histobar—nondystrophic, gray histobar—mdx. N is the number of mice and corresponding muscle preparations. ** indicates p<0.01 in comparison to nondystrophic mean. Immunoprecipitation of cytosolic IκB-α revealed the presence of a sumoylated IκB-α (FIG. 49A2) that had previously been reported in a variety of cell lines. Desterro et al. (1998). These results indicate that SUMO binds to cytosolic IκB-α in skeletal muscle (FIG. 49A2) and may contribute to the elevated IκB-α expression in mdx cytosol (compare black histobars of FIGS. 48B2 and 48C2).

### Mdx Costal Diaphragms Exhibit Increases in the Proportion of Phosphorylated Cytosolic Akt

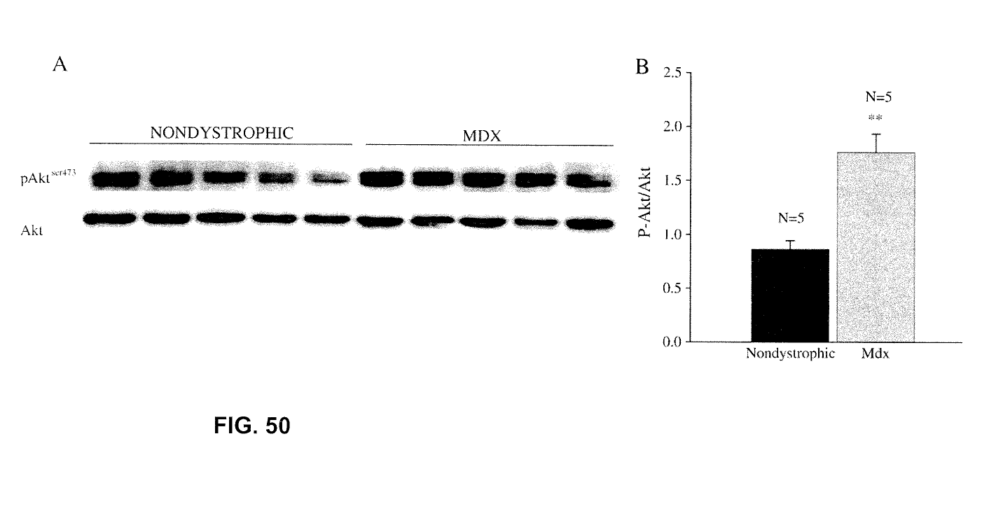

Increases in the activation of the Akt pathway have been reported in the mdx and mdx-utr-/-mouse and in Duchenne muscular dystrophy. See Peter et al. (2006). Since increased phosphorylation of Akt could contribute to enhanced activation of the NF-κB pathway, the phosphorylation status of Akt was examined in whole cell lysates from nondystrophic and mdx costal diaphragms. Manning and Cantley (2007). Mdx costal diaphragm lysates exhibited significantly (p<0.01) higher proportions of phosphorylated Akt that were approximately double those observed in nondystrophic preparations (FIG. 50). FIG. 50 shows increases in whole cell Akt phosphorylation in mdx costal diaphragm. (A) Western blots of phospho-Akt (pAktser473, top row) and Akt (bottom row) in whole cell lysates from 3.5 month old nondystrophic (left side) and mdx (right side) costal diaphragms. (B) Proportion of phosphorylated Akt in nondystrophic (black histobar) and mdx (gray histobar) costal diaphragms. Shown are the means and standard errors. N is the number of mice and corresponding muscle preparations. ** indicates p<0.01 in comparison to nondystrophic muscle.

Attempts to reduce Akt phosphorylation in mdx muscle by in vivo exposure to the Akt inhibitor triciribine (1 mg/kg, daily, intraperitoneal, 6 days treatment; Yang et al. (2004)), or dexamethasone (1 mg/kg; intraperitoneal, daily, 21 days treatment) which blocks IGF receptor activation upstream of Akt in skeletal muscle, were ineffective at reducing the proportion of phosphorylated Akt in the mdx costal diaphragm. See Sandri et al. (2004).

As shown in the studies above,the expression level of p65 was substantially increased in dystrophic muscle as compared to that of normal tissues. This observation indicates that treatments to reduce NF-κB activation by stabilizing cytosolic IκB-α and therefore reducing the proportion of the total cellular p65 residing in the nucleus, will not be as effective as treatments to reduce the cellular expression (production) of p65, or treatments to reduce or inhibit p65 transactivation of the NF-κB promoter (Singh et al., 2009). Therefore, at least two approaches can be taken to find new therapeutic agents for treating MD. One approach is to use a therapeutic agent to inhibit p65 transactivation. The other approach is to use a therapeutic agent to reduce the cellular expression of p65.

### Example 16

### Treating Muscular Dystrophy with Agents that Reduce p65 Transactivation

Many agents, including chemicals, polynucleotides, or polypeptides, have been identified as inhibitors of NF-κB transactivation (also called “NF-κB transactivation inhibitors”). These agents typically inhibit NF-κB transactivation by competing for DNA binding sites on target genes, or by competing against NF-κB for binding to other co-factors that are essential for NF-κB transactivation. One such inhibitor is ursodeoxycholic acid (UDCA). Miura et. al. (2001). Other inhibitors may include, for example, the peptide inhibitors as taught in U.S. Pat. No. 7,368,430.

In one experiment, the capabilities of ursodeoxycholic acid (UDCA) and pyrrolidine dithiocarbamate (PDTC; Siegel et al., 2009) to suppress p65 activation were compared (Example 14). PDTC is known to be an IκB-α stabilizing agent that inhibits the nuclear translocation of NF-κB, while UDCA has been shown to reduce NF-κB transactivation in several mammalian cell lines by a mechanism that involves interaction with glucocorticoid receptors (GR), nuclear translocation of the GR-UDCA complex, and inhibition of p65 transactivation. Miura et. al. (2001). UDCA was more effective in suppressing NF-κB activation in the mdx diaphragm than PDTC. This study also shows that UDCA produced larger improvements in limb muscle function than PDTC.

Different compositions containing either UDCA alone, or in conjunction with certain inhibitors of IKKβ, or other agents that inhibit the nuclear translocation of NF-κB, may be administered intravenously to treat MD. Agents that reduce the cellular expression of p65 may also be administered in conjunction with the various NF-κB inhibitors.

### Example 17

### Treating Muscular Dystrophy with Agents that Reduce p65 Expression Levels

Various agents that reduce the expression of p65 are administered alone or in combination to a subject with MD (also called “Inhibitors of p65 expression” in this disclosure). These agents may act at the transcription level by decreasing the transcription of p65 gene, or at the translation levels by suppressing the translation of p65 mRNA into p65 protein. The agents may also alter the stability of the p65 mRNA or p65 protein thus reducing the levels of p65 protein.

In one aspect, one such agent, gossypol, is administered to a subject with MD. Gossypol is usually isolated from cottonseed, and has been reported to decrease constitutive NF-κB activation in human leukemic cells. Gossypol is known to decrease nuclear expression of the transcription factor Sp1 and the binding of Sp1 to DNA, an effect which was associated with significant reductions in cytosolic and nuclear p65 expression and reductions in p65 mRNA levels (Moon et al, 2008).

In one embodiment, siRNAs that complement the p65 mRNA may also be administered to an subject with MD to reduce the expression of the p65 gene in viva In one aspect, methods similar to the ones described in Duan et al. (2007) for inhibiting p65 expression using siRNA may be employed to suppress p65 expression in the muscular tissues of a subject, such as a human or a mouse. In another aspect, the octa-guanidine adjunct (“vivo-morpholinos”) methodology may be used to facilitate tissue delivery following intraperitoneal or intravascular administration (Wu et al., 2009). These agents may be used alone, or in combination with other NF-κB inhibitors that operate under different mechanisms.

### REFERENCES

Acharyya S, Villalta A, Bakkar N, Bupha-Intr T, Janssen P M L, Carathers M, et al. Interplay of IKK/NF-κB signaling in macrophages and myofibers promotes muscle degeneration in Duchenne muscular dystrophy. J Clin Invest 2007; 117:889-901.
Almawi, W. Y. and O. K. Melemedjian, Negative regulation of nuclear factor-κB activation and function by glucocorticoids, J. Mol. Endocrinol. 28 (2002), pp. 69-78.
Andrade, F. H., J. D. Porter and H. J. Kaminski, Eye muscle sparing by the muscular dystrophies: lessons to be learned?, Microsc. Res. Tech. 48 (2000), pp. 192-203.
Arahata, K., S. Ishiura, T. Ishiguro, T. Tsukahara, Y. Suhara, C. Eguchi, T. Ishihara, I. Nonaka, E. Ozawa and H. Sugita, Immunostaining of skeletal and cardiac muscle surface membrane with antibody against Duchenne muscular dystrophy peptide, Nature 333 (1988), pp. 861-863.
Araki, S. and S. Mawatari, Ouabain and erythrocyte-ghost adenosine triphosphatase, Arch. Neurol. 24 (1971), pp. 187-190.
Ariga, A., J.-I. Namakawa, N. Matsumoto, J.-I. Inoue and K. Umezawa, Inhibition of tumor necrosis factor α-induced nuclear translocation and activation of NFκB by dehydroxymethylepoxyquinomicin, J. Biol. Chem. 277 (2002) (27), pp. 24625-24630.
Barnes, P. J., Molecules in focus: nuclear factor-κB, Int. J. Biochem. Cell Biol. 29 (1997) (6), pp. 867-870.
Blake, D. J., A. Weir, S. E. Newey and K. E. Davies, Function and genetics of dystrophin and dystrophin-related proteins in muscle, Physiol. Rev. 82 (2001), pp. 291-329.
Blondeau, N., C. Widmann, M. Lazdunski and C. Heurteaux, Activation of nuclear factor-κB is a key event in brain tolerance, J. Neurosci. 21 (2001) (13), pp. 4668-4677.
Broad, L. M., T. R. Cannon and C. W. Taylor, A non-capacitative pathway activated by arachidonic acid is the major Ca2+ entry mechanism in rat A7r5 smooth muscle cells stimulated with low concentrations of vasopressin, J. Physiol. 517 (1999), pp. 121-134.
Bulfield, G., W. G. Siller, P. A. L. Wright and K. J. Moore, X-chromosome-linked muscular dystrophy (mdx) in the mouse, Proc. Natl. Acad. Sci. U.S.A. 81 (1984), pp. 1189-1192.
Cai, D., J. D. Frantz, N. E. Tawa Jr., P. A. Melendez, B.-C. Oh, H. G. W. Lidov, P.-O. Hasselgren, W. R. Frontera, J. Lee, D. J. Glass and S. E. Shoelson, IKKβ/NF-κB activation causes severe muscle wasting in mice, Cell 119 (2004), pp. 285-298.
Carlson, C. G., The dystrophinopathies: an alternative to the structural hypothesis, Neurobiol. Dis. 5 (1998), pp. 3-15.
Carlson, C. G. and T. Geisbuhler, Diastolic calcium influx in isolated rat cardiac myocytes, Abstracts from the XXVth Annual Meeting, North American Section of the International Society for Heart Research, J. Mol. Cell. Cardiol. vol. 35 (6) (2003).
Carlson, C. G. and R. V. Makiejus. “A noninvasive procedure to detect muscle weakness in the mdx mouse.” Muscle and Nerve (1990) 13:480-484.
Carlson, C. G. and D. Roshek, Adult dystrophic (mdx) endplates exhibit reduced quantal sensitivity and enhanced quantal variation, Pflugers Arch. (Eur. J. Physiol.) 442 (2001), pp. 369-375.
Carlson, C. G., R. Ashmore, A. Gueorguiev, D. Roshek and J. Chu, Manganese quench rates and steady state calcium concentrations in cultured dystrophic myotubes and freshly excised adult dystrophic fibers, Neurosci. Abstr. vol. 27 (2001) Program number 105.14.
Carlson, C. G., A. Gueorguiev, D. M. Roshek, R. Ashmore, J. S. Chu and J. E. Anderson, Extra-junctional resting Ca2+ influx is not increased in a severely dystrophic expiratory muscle (triangularis sterni) of the mdx mouse, Neurobiol. Dis. 14 (2003) (2), pp. 229-239.
Carlson C G, Samadi A, Siegel A. Chronic treatment with agents that stabilize cytosolic IκBα enhance survival and improve resting potential in mdx muscle fibers subjected to chronic passive stretch. Neurobiol Dis 2005; 20:719-30.
Chakkalakal, J. V., M.-A. Harrison, S. Carbonetto, E. Chin, R. N. Michel and B. J. Jasmin, Stimulation of calcineurin signaling attenuates the dystrophic pathology in mdx mice, Hum. Mol. Genet. 13 (2004) (4), pp. 379-388.
Cox, J. L., T. Lancaster and C. G. Carlson, Changes in the motility of B16F10 melanoma cells induced by alterations in resting calcium influx, Melanoma Res. 12 (2002), pp. 211-219.
Cullen, M. J. and J. J. Fulthorpe, Stages in fibre breakdown in Duchenne muscular dystrophy: an electron-microscopic study, J. Neurol. Sci. 24 (1975), pp. 179-200.
Cuzzocrea, S., P. K. Chatterjee, E. Mazzon, L. Dugo, I. Serraino, D. Britti, G. Mazzullo, A. P. Caputi and C. Thiemermann, Pyrrolidine dithiocarbamate attenuates the development of acute and chronic inflammation, Br. J. Pharmacol. 135 (2002), pp. 496-510.
D'Acquisto, F. D., A. lalenti, T. luvone, M. DiRosa and R. Carnuccio, Inhibition of nuclear factor-κB prevents the loss of vascular tone in lipopolysaccharide-treated rats, Eur. J. Pharmacol. 365 (1999), pp. 253-257.
D'Acquisto, F. D., A. lanaro, A. lalenti, P. Maffia, M. C. Maiuri and R. Carnuccio, Transcription factor decoy oligodeoxynucleotides to nuclear factor-κB inhibit reverse passive Arthus reaction in rat, Naunyn-Schmiedeberg's Arch. Pharmacol. 364 (2001), pp. 422-429.
DeBacker, F., C. Vandebrouk, P. Gailly and J. M. Gillis, Long-term study of Ca2+ homeostasis and of survival in collagenase-isolated muscle fibres from normal and mdx mice, J. Physiol. (London) 542.3 (2002), pp. 855-865.
DeBosscher, K., W. V. Berghe and G. Haegeman, The interplay between the glucocorticoid receptor and nuclear factor-κB or activator protein-1: molecular mechanisms for gene repression, Endocr. Rev. 24 (2003) (4), pp. 488-522.
Deconinck N, Tinsley J, De Backer F, Fisher R, Kahn D, Phelps S, et al. Expression of truncated utrophin leads to major functional improvements in dystrophin deficient muscles of mice. Nat Med 1997; 11:1216-21.
Desterro J M P, Rodriguez M P, Hay RT . SUMO-1 modification of IκBα inhibits NF-κB activation. Mol Cell 1998; 2:233-9.
DeTroyer, A. and A. Legrand, Mechanical advantage of the canine triangularis sterni, J. Appl. Physiol. 84 (1998) (2), pp. 562-568.
DeTroyer, A. and V. Ninane, Triangularis sterni: a primary muscle of breathing in the dog, J. Appl. Physiol. 60 (1986), pp. 14-21.
DeTroyer, A., A. Legrand, P.-A. Gevenois and T. A. Wilson, Mechanical advantage of the human parasternal intercostal and triangularis sterni muscles, J. Physiol. (London) 513.3 (1998), pp. 915-925.
Duan J, Friedman J, Nottingham L, Chen Z, Ara G, Van Waes C., Nuclear factor-kappaB p65 small interfering RNA or proteasome inhibitor bortezomib sensitizes head and neck squamous cell carcinomas to classic histone deacetylase inhibitors and novel histone deacetylase inhibitor PXD101. Mol Cancer Ther. 2007 January; 6(1):37-50.
Durham W J, Arbogast S, Gerken E, Li Y-P, Reid M B. Progressive nuclear factor-kB activation resistant to inhibition by contraction and curcumin in mdx mice. Muscle Nerve 2006; 34:298-303.
El-Bohy, A. A. and B. L. Wong, The diagnosis of muscular dystrophy, Pediatr Ann. (2005) 34(7):525-30.
Ervasti, J. M. and K. P. Camp<ell, Membrane organization of the dystrophin-glycoprotein complex, Cell 66 (1991), pp. 1121-1131.
Fenichel, G. M., J. M. Florence, A. Pestronk, J. R Mendell, R. T. Moxley III, R. C. Griggs, M. H. Brooke, J. P. Miller, J. Robison, W. King, L. Signore, S. Pandya, J. Schierbecker and B. Wilson, Long-term benefit from prednisone therapy in Duchenne muscular dystrophy, Neurology 41 (1991), pp. 1874-1877.
Franco Jr., A., B. D. Winegar and J. B. Lansman, Open channel block by gadolinium ion of the stretch-inactivated ion channel in mdx myotubes, Biophys. J. 59 (1991), pp. 1164-1170.
Gan et al., J. of Gastroenterology and Hepatology, (2005) 20:1016-24.
Gosselin, L. C., J. E. Barkley, M. J. Spencer, K. M. McCormick and G. A. Farkas, Ventilatory dysfunction in mdx mice: impact of tumor necrosis factor-alpha deletion, Muscle Nerve 28 (2003), pp. 336-343.
Guttridge, D. C., M. W. Mayo, L. V. Madrid, C.-Y. Wang and A. S. Baldwin Jr., NF-κB-induced loss of Myo D messenger RNA: a possible role in muscle decay and cachexia, Science 289 (2000), pp. 2363-2366.
Hayden M S, Ghosh S. Shared principles in NF-κB signaling. Cell 2008; 32(3):344-62.
Hoffman, E. P., R. H. Brown and L. M. Kunkel, Dystrophin: the protein product of the Duchenne muscular dystrophy locus, Cell 51 (1987), pp. 919-928.
Hudecki M S, Pollina C M, Granchelli J A, Daly M K, Byrnes T, Wang J C, et al. Strength and endurance in the therapeutic evaluation of prednisolone—treated mdx mice. Res Commun Chem Pathol Pharmacol 1993; 79:45-60.
Hwang, J.-C., D. Zhou and W. M. St. John, Characterization of expiratory intercostal activity to triangularis sterni in cats, J. Appl. Physiol. 67 (1989) (4), pp. 1518-1524.
Jinni, E., K. Aoki, H. Saito, F. D. D'Acquisto, M. J. May, I. Nakamura, T. Sudo, T. Kojima, F. Okamoto, H. Fukushima, K. Okabe, K. Ohya and S. Ghosh, Selective inhibition of NF-κB blocks osteoclastogenesis and prevents inflammatory bone destruction in vivo, Nat. Med. 10 (2004) (6), pp. 617-624.
Jonsdottir, I. H., P. Scherling, K. Ostrowski, S. Asp, E. A. Richter and B. K. Pedersen, Muscle contractions induce interleukin-6 mRNA production in rat skeletal muscles, J. Physiol. 528.1 (2000), pp. 157-163.
Karin, M., Y. Yamamoto and Q. M. Wang, The IKK NF-κB system: a treasure trove for drug development, Nat. Rev. 3 (2004), pp. 17-26.
Koenig, M. and L. M. Kunkel, Detailed analysis of the repeat domain of dystrophin reveals four potential hinge segments that may confer flexibility, J. Biol. Chem. 265 (1990), pp. 4560-4566.
Kreydiyyeh, S. I. and R. Al-Sadi, Interleukin-1 beta increases urine flow rate and inhibits protein expression of Na+/K+ ATPase in the rat jejunum and kidney, J. Interferon Cytokine Res. 22 (2002) (10), pp. 1041-1048.
Kreydiyyeh, S. I., C. Abou-Chahine and R. Hilal-Dandan, Interleukin-1 beta inhibits Na+-K+ ATPase activity and protein expression in cardiac myocytes, Cytokine 26 (2004) (1), pp. 1-8.
Kumar, A. and A. M. Boriek, Mechanical stress activates the nuclear factor-kappa B pathway in skeletal muscle fibers: a possible role in Duchenne muscular dystrophy, FASEB J. 17 (2003), pp. 296-386.
Lang, C. H., C. Silvis, N. Deshpande, G. Nystrom and R. A. Frost, Endotoxin stimulates in vivo expression of inflammatory cytokines tumor necrosis factor alpha, interleukin-1beta,-6, and high mobility-group protein-1 in skeletal muscle, Shock 19 (2003) (6), pp. 538-546.
Lappas et al., Endocrinology, (2005) 146 (3), 1491-1497.
Li, X., P. E. Massa, A. Hanidu, G. W. Peet, P. Aro, A. Savitt, S. Mische, J. Li and K. B. Marcu, IKKα, IKKβ, and NEMO/IKKγ are each required for the NF-κB-mediated inflammatory response program, J. Biol. Chem. 277 (2002) (47), pp. 45129-45140.
Lloyd, S. J. and A. E. H. Emery, A possible circulating plasma factor in Duchenne muscular dystrophy, Clin. Chim. Acta 112 (1981), pp. 85-90.
Makiejus R V, Patel V K, Krishna G, Dierdorf S F, Bonsett C. Effect of adenylosuccinate on the strength of dystrophin lacking muscles of mdx mice. Biochem Arch 1991; 7:95-103.
Manning B D, Cantley L C. Akt/PKB signaling: navigating downstream. Cell 2007; 129:1261-74.
Matsuda, R., A. Nishikawa and H. Tanaka, Visualization of dystrophic muscle fibers in mdx mouse by vital staining with Evans blue: evidence of apoptosis in dystrophin-deficient muscle, J. Biochem. 118 (1995), pp. 959-964.
Matsumura, K. and K. P. Camp<ell, Dystrophin-glycoprotein complex: its role in the molecular pathogenesis of muscular dystrophies, Muscle Nerve 17 (1994), pp. 2-15.
Messina S, Bitto A, Aguennouz M, Minutoli L, Monici MC, Altavilla D, et al. Nuclear factor kappa-B blockade reduces skeletal muscle degeneration and enhances muscle function in Mdx mice. Exp Neurol 2006; 198: 234-41.
Millman G W, Turin E C, Samadi A, Carlson C G. The NF-kappaB pathway in dystrophic (mdx) skeletal muscle produces elevated active p65 in the nuclear compartment without altering the subcellular distribution of the transcription factor. American Society Cell Biology Abstracts 2007; #1823:499.
Miura T, Ouchida R, Yoshikawa N, Okamoto K, Makino Y, Nakamura T, Morimoto C, Makino I, Tanaka H. Functional modification of the glucocorticoid receptor and suppression of NF-kappa-B dependent transcription by ursodeoxycholic acid. J. Biol. Chem. 2001; 276 (50): 47371-47378.
Monici, M. C., M. Aguennouz, A. Mazzeo, C. Messina and G. Vita, Activation of nuclear factor-κB in inflammatory myopathies and Duchenne muscular dystrophy, Neurology 60 (2003), pp. 993-997.
Moon D-O, Kim M-O, Lee J-D, Kim G-Y. Gossypol suppresses NF-κB activity and NF-κB-related gene expression in human leukemia U937 cells, Cancer Letters, 264 (2), 192-200, 2008.

Nagel, A., F. Lehmann-Horn and A. G. Engel, Neuromuscular transmission in the mdx mouse, Muscle Nerve 13 (1990), pp. 742-749.
Ninane, V., R. E. Baer and A. De Troyer, Mechanism of triangularis sterni shortening during expiration in dogs, J. Appl. Physiol. 66 (1989) (5), pp. 2287-2292.
Orlowski, R. Z. and A. S. Baldwin Jr., NF-κB as a therapeutic target in cancer, Trends Mol. Med. 8 (2002) (8), pp. 385-389.
Peter, J. B., M. Worsfold and C. M. Pearson, Erythrocyte ghost adenosine triphosphatase (ATPase) in Duchenne dystrophy, J. Lab. Clin. Med. 74 (1969) (1), pp. 103-108.
Peter A K, Crosbie R. Hypertrophic response of Duchenne and limb-girdle muscular dystrophies is associated with activation of Akt pathway. Exptl Cell Res 2006; 312: 2580-91.
Petrof, B. J., J. B. Shrager, H. H. Stedman, A. M. Kelly and H. L. Sweeney, Dystrophin protects the sarcolemma from stresses developed during muscle contraction, Proc. Natl. Acad. Sci. U.S.A. 90 (1993), pp. 3710-3714.
Rangan, G. K., Y. Wang, Y.-C. Tay and D. C. H. Harris, Inhibition of nuclear factor-κB activation reduces cortical tubulointerstitial injury in proteinuric rats, Kidney Int. 56 (1999), pp. 118-134.
Robe et al., Clinical Cancer Research, (2004)10, 5595-5603.
Sakakibara, H., A. G. Engel and E. H. Lambert, Duchenne dystrophy: ultrastructural localization of the acetylcholine receptor and intracellular microelectrode studies of neuromuscular transmission, Neurology 27 (1977), pp. 741-745.
Samadi, A., R. J. Cenedella and C. G. Carlson, Diethylstillbestrol (DES) increases intracellular calcium in lens epithelial cells, Pfluegers Arch. (Eur. J. Physiol.) (2005).
Sandri M, Sandri C, Gilbert A, Skurk C, Calabria E, Picard A, et al. Foxo transcription factors induce the atrophy-related ubiquitin ligase atrogin-1 and cause skeletal muscle atrophy. Cell 2004; 117:399-412.
Satoh, A., T. Shimosegewa, M. Fujita, A. Masamune, M. Koizumi and T. Toyota, Inhibition of nuclear factor-κB activation improves the survival of rats with taurocholate pancreatitis, Gut 44 (1999), pp. 253-258.
Siebenlist, U., G. Franzoso and K. Brown, Structure, regulation and function of NF-κB, Annu. Rev. Cell Biol. 10 (1994), pp. 405-455.
Siegel A L, Bledsoe C, Lavin J, Gatti F, Berge J, Millman G, Turin E, Winders W T, Rutter J, Palmeiri B, Carlson C G. Treatment with inhibitors of the NF-κB pathway improves whole body tension development in the mdx mouse. Neuromuscular Disorders (2009) 19: 131-139.
Singh, S. and B. B. Aggarwal, Activation of transcription factor NF-κB is suppressed by curcumin (diferulolylmethane), J. Biol. Chem. 270 (1995) (42), pp. 24995-25000.
Singh R, Millman G, Turin E, Polisiakeiwicz L, Lee B, Gatti F, Berge J, Smith E, Rutter J, Sumski C, Winders W T, Samadi A, Carlson C G, Increases in nuclear p65 activation in dystrophic skeletal muscle are secondary to increases in the cellular expression of p65 and are not solely produced by increases in IκB-α kinase activity. J. of Neurological Sciences, 2009 (In press).
Takeuchi, T., S. Miura, L. Wang, K. Uehara, M. Mizumori, H. Kishikawa, R. Hokari, H. Higuchi, M. Adachi, H. Nakamizo and H. Ishii, Nuclear factor-κB and TNF-α mediate gastric ulceration induced by phorbol myristate acetate, Dig. Dis. Sci. 47 (2002), pp. 2070-2078.
Thaloor, D., K. J. Miller, J. Gephart, P. O. Mitchell and G. K. Pavlath, Systemic administration of the NF-κB inhibitor curcumin stimulates muscle regeneration after traumatic injury, Am. J. Physiol.: Cell Physiol. 46 (1999), pp. C320-C329.
Tutdibi, O., H. Brinkmeir, R. Rudel and K. J. Fohr, Increased calcium entry into dystrophin-deficient muscle fibers of MDX and ADR-MDX mice is reduced by ion channel blockers, J. Physiol. (London) 515.3 (1999), pp. 859-868.
Wahl et al., J. Clin. Invest., (1998) 101, 1163-74.
Weber et al., Gastroenterology, (2000) 119, 1209-18.
Weller, B., G. Karpati and S. Carpenter, Dystrophin-deficient mdx muscle fibers are preferentially vulnerable to necrosis induced by experimental lengthening contractions, J. Neurol. Sci. 100 (1990), pp. 9-13.
Wu B, Li Y, Morcos P A, Doran T J, Lu P, Lu Q L, Octa-guadinine morpholino restores dystrophin expression in cardiac and skeletal muscles and ameliorates pathology in dystrophic mdx mice, Molecular Therapy, doi:10.1038/mt.2009.38.
Yang, X. C. and F. Sachs, Block of stretch-activated ion channels in Xenopus oocytes by gadolinium and calcium ions, Science 243 (1989), pp. 1068-1071.
Yang L, Dan H C, Sun M, Liu Q, Sun X M, Feldman R I, et al. Akt/Protein kinase B signaling inhibitor-2, a selective small molecule inhibitor of Akt signaling with antitumor activity in cancer cells overexpressing Akt. Cancer Res 2004; 64:4394-9.
Yeung, E. W., S. I. Head and D. G. Allen, Gadolinium reduces short-term stretch-induced muscle damage in isolated mdx mouse muscle fibres, J. Physiol. (London) 552.2 (2003), pp. 449-458.
Yuksel, M., K. Okajima, M. Uchiba and H. Okabe, Gabaxate mesilate, a synthetic protease inhibitor, inhibits lipopolysaccharide-induced tumor necrosis factor-α production by inhibiting activation of both nuclear factor-κB and activator protein-1 in human monocytes, J. Pharmacol. Exp. Ther. 305 (2003), pp. 298-305.

We claim:

1. A method for treating muscular dystrophy in a subject, said method comprising the steps of
diagnosing a subject with Duchenne muscular dystrophy, and
administering to the subject an agent in an amount effective for decreasing the level or the activity of the p65 subunit of NFκB in the muscular tissues of the subject.

2. The method of claim 1, wherein the agent is at least one member selected from the group consisting of NF-κB transactivation inhibitors, p65 expression inhibitors, IκB stabilizing agent, Ikk inhibitors and combination thereof.

3. The method of claim 1 wherein the agent is an inhibitor of p65 expression.

4. The method of claim 3 wherein the agent decreases the translation from p65 mRNA into p65 protein.

5. The method of claim 4 wherein the agent is a small interfering RNA (siRNA) that complements the coding sequence of the p65 subunit of NFκB.

6. The method of claim 3 wherein the agent decreases the transcription of the p65 gene into p65 mRNA.

7. The method of claim 4 where the agent is a specific translation blocking vivo-morpholino to reduce the translation and synthesis of p65 in dystrophic muscle following either in vitro or in vivo exposure.

8. The method of claim 1 where the agent reduces cellular p65 levels by de-stabilizing cellular p65 in dystrophic tissues thereby promoting its degradation and/or cytosolic localization.

9. The method of claim 8 where the agent is an inhibitor of the peptidyl prolyl isomerase (PIN-1) that stabilizes the expression of p65 in dystrophic muscle fibers.

10. The method of claim 1 wherein the agent decreases the transactivation activity of the p65 subunit of NFκB in the muscular tissues of the subject.

11. The method of claim 1 wherein the agent is ursodeoxycholic acid (UDCA).

12. The method of claim 1 wherein the agent is administered as a pharmaceutical composition.

13. The method of claim 1 further comprising the step of monitoring NFκB levels in the subject to ascertain the effect of treatment.

14. The method of claim 1, wherein the agent is administered at an amount sufficient to decrease the level or the activity of the p65 subunit of NFκB by at least 30% in the muscular tissues of the subject.

15. The method of claim 1, wherein the agent is administered at an amount sufficient to decrease the level or the activity of the p65 subunit of NFκB by at least 50% in the muscular tissues of the subject.

16. The method of claim 1 wherein the agent is pyrrolidine dithiocarbamate (PDTC).

17. A method for treating muscular dystrophy in a subject, said method comprising the step of administering to the subject an agent in an amount effective for decreasing the level or the activity of the p65 subunit of NFκB in the muscular tissues of the subject, wherein said agent comprises ursodeoxycholic acid (UDCA).

18. The method of claim 17, wherein the agent is administered at an amount sufficient to decrease the level or the activity of the p65 subunit of NFκB by at least 30% in the muscular tissues of the subject.

19. The method of claim 17 wherein the agent is administered together with at least one other pharmaceutically inactive ingredient.

20. The method of claim 17 further comprising the step of monitoring NFκB levels in the subject to ascertain the effect of treatment.

21. A method for treating muscular dystrophy in a subject, said method comprising the step of administering to the subject an agent in an amount effective for decreasing the level or the activity of the p65 subunit of NFκB in the muscular tissues of the subject, wherein said agent comprises pyrrolidine dithiocarbamate (PDTC).

22. The method of claim 21 wherein the agent is administered together with at least one other pharmaceutically inactive ingredient.

23. The method of claim 21 further comprising the step of monitoring NFκB levels in the subject to ascertain the effect of treatment.
